(19) United States
(12) Patent Application Publication
(10) Pub. No.: US 2023/0127949 A1
(43) Pub. Date: Apr. 27, 2023

(54) PURIFICATION OF RECOMBINANTLY PRODUCED POLYPEPTIDES

(71) Applicant: Novartis AG, Basel (CH)

(72) Inventors: Anne-Sophie BLUEMMEL, Freiburg (DE); Hervé MOEBEL, Buschwiller (FR); Anders Klas PALM, Binningen (FR); Isabelle SAVOY, Weil am Rhein (DE); Henry STOSCH, Basel (CH)

(21) Appl. No.: 17/759,943

(22) PCT Filed: Feb. 22, 2021
(86) PCT No.: PCT/IB2021/051484

§ 371 Date: Aug. 2, 2022

Related U.S. Application Data
(60) Provisional application No. 62/980,630, filed on Feb. 24, 2020.

(51) Int. Cl. C07K 1/36; C07K 14/47; B01D 15/38; B01D 15/36; B01D 15/32; B01D 15/18

(52) U.S. Cl. CPC C07K 1/36; C07K 14/4725; B01D 15/3847; B01D 15/362; B01D 15/363; B01D 15/327; B01D 15/1871; A61K 38/00

(57) ABSTRACT

The present invention relates generally to processes for production of heavily glycosylated recombinant proteins (e.g., mucins and mucin-like proteins, such as lubricin), the processes comprising culturing mammalian cells capable of producing a glycoprotein in a liquid medium in a system comprising one or more bioreactors, concentrating and purifying and formulating the glycoprotein, the purification comprising one or more steps of chromatography, an endonuclease step, and at least one step of viral inactivation. In certain aspects the invention relates to pharmaceutical compositions comprising purified recombinant human lubiricin, and methods of treating a subject in need thereof.

### CROSS-REFERENCE TO RELATED APPLICATIONS

This application claims the benefit of and priority to U.S. patent application Ser. No. 62/980,630, filed Feb. 24, 2020, which is herein incorporated by reference in its entirety, for all purposes.

### SEQUENCE LISTING

The instant application contains a Sequence Listing which has been submitted electronically in ASCII format and is hereby incorporated by reference in its entirety. Said ASCII copy, created on Jan. 29, 2021, is named PAT058776-WO-PCT_SL.txt and is 24,738 bytes in size.

### FIELD OF THE INVENTION

The present invention relates to processes for production and purification of heavily glycosylated recombinant proteins, and compositions comprising purified heavily glycosylated recombinant proteins.

### BACKGROUND

Lubricin or PRG4, a product of the proteoglycan 4 (PRG4) gene, is highly expressed by synoviocytes and superficial zone chondrocytes (Rhee D K et al., J Clin Invest. 2005 March; 115(3):622-31). Lubricin is a glycoprotein that functions as a critical boundary lubricant for articular cartilage and normally isolated from synovial fluid (Swann D A et al, J Biol Chem. 1981 Jun. 10; 256(11):5921-5).

Lubricin is expressed from the PRG4 gene with a full length spanning 12 exons, although multiple, naturally occurring truncated versions have been reported. The lubricin molecule is a long, flexible molecule with a fully extended “contour” length of lc≈200 nm and a diameter of a few nanometers. Its molecular weight is approximately Mw≈280-320 kDa. The central portion of the molecule, known as the “mucin domain”, is highly glycosylated (Jay, G. et al., Glycoconjugate J. 2001, 18 (10), 807-815). Within this mucin domain, short glycan oligomers terminated primarily by polar galactose (˜33% of total glycans) and negatively charged sialic acid (˜66% of total glycans) are O-linked to threonine and serine residues (Jay, G.et al.,Glycoconjugate J. 2001, 18 (10), 807-815; Estrella, R. P. et al., Biochem. J. 2010, 429 (2), 359-367). With abundant negatively charged and highly hydrated sugar groups, this central mucin domain is believed to be responsible for both lubricin's lubrication as well as antiadhesive properties (Aninwene, G. E. et al., J. Biomed. Mater. Res., Part A 2015, 103 (2), 451-462; Greene, G. et al., Biomaterials 2015, 53 (0), 127-136). Flanking either end of the mucin domain are the lightly glycosylated “end domains” of the protein which contain sub-domains similar to two globular proteins, somatomedin-B and homeopexin, known to play a special role in cell-cell and cell-extracellular matrix interactions, e.g., binding (Jay, G. et al., Glycoconjugate J. 2001, 18 (10), 807-815; Estrella, R. P. et al., Biochem. J. 2010, 429 (2), 359-367). These end domains are extremely “sticky” and are able to adhere to nearly all types of surfaces. These end domains have also been shown to associate with each other to form molecular “loops” and also allow the lubricin to easily form dimers, trimers, and tetramers and larger, loosely twisted aggregate structures (Zappone, B. et al., Langmuir 2008, 24 (4), 1495-1508).

There is a large pharmaceutical and scientific interest in lubricin. Lubricin has been proposed for administration by injection into the synovium to slow the worsening of arthritis symptoms. See, e.g., U.S. Pat. No. 8,026,346 and published application number US 20090104148. Patent application publication number US 20130116186 discloses injection of lubricin into asymptomatic joints at risk of developing arthritis so as to preserve and enhance joint lubrication, preserve chondrocytes and promote healthy expression of the endogenous lubricin they produce. Lubricin also has been proposed for use as a topical treatment for dry eye disease, and as a treatment for interstitial cystitis, among other uses.

For human application, every pharmaceutical substance has to meet distinct criteria. Preferably, biopharmaceutical products have a very high purity, with the concentration of impurities, such as host cell proteins and nucleic acids (e.g., DNA), reduced to the range of parts per million relative to the desired product, or lower. To meet the regulatory specifications, one or more purification steps have to follow the manufacturing process. Among others, purity, throughput, and yield play important roles in determining an appropriate purification process. Previous attempts at manufactunng and purification of recombinant lubricin at a scale suitable for commercial pharmaceutical exploitation either have not been successful or yielded inferior results. It is a challenge to produce and purify the recombinantly expressed lubricin product and its multimeric complexes from contaminants while retaining its lubrication and other functions, while avoiding aggregation and fragmentation and maintaining high yield. Purification of lubricin is particularly difficult due to the heavy glycosylation of lubricin, its abundant negatively charged and highly hydrated sugar groups in the central mucin domain and extremely “sticky” end domains, its high molecular weight, and its tendency to form complexes and to aggregate to form insoluble microparticles as purity increases. A purification method of recombinant lubricin has been described in published application number US20160304572. However, there remains a need for an improved purification process of recombinant lubricin that optimizes removal of impurities, in particular, host cell proteins and DNA, and that is suitable for commercial exploitation.

### SUMMARY

In specific aspects, it is an object of the present invention to provide a purification process of separating recombinantly expressed lubricin glycoprotein product and its multimeric complexes from contaminants, which enables efficient purification of lubricin from impurities while retaining its biological functions, further avoiding aggregation, and maintaining high yield of the final lubricin protein product. It is also an object of the present invention to provide recombinant lubricin and compositions thereof, for example, pharmaceutically acceptable compositions of recombinant lubricin, purified using methods described herein.

In the first aspect, the current invention relates to a method of purifying a recombinantly produced glycosylated polypeptide, in particular a recombinantly produced glycosylated lubricin glcyoprotein, wherein the method comprises three chromatography steps: (a) a multimodal cation exchange chromatography (MCC) step; (b) a multimodal anion exchange chromatography (MAC) step; and (c) a hydrophobic interaction chromatography (HIC) step. In a specific embodiment, the current invention relates to a method of purifying a recombinantly produced glycosylated polypeptide, in particular a recombinantly produced glycosylated lubricin, wherein the method comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC).

In the second aspect, the current invention relates to a method of reducing contaminants (e.g., polynucleotide and/or host cell protein contamination) in a formulation comprising a recombinantly produced glycosylated polypeptide, in particular a recombinantly produced glycosylated lubricin glycoprotein, wherein the method: (i) comprises three chromatography steps: (a) a multimodal cation exchange chromatography (MCC) step; (b) a multimodal anion exchange chromatography (MAC) step; and (c) a hydrophobic interaction chromatography (HIC) step; and (ii) further comprises a step of preparing a formulation comprising the recovered recombinantly produced polypeptide. In a specific embodiment, the current invention relates to a method of reducing contaminants (e.g., polynucleotide and/or host cell protein contamination) in a formulation comprising a recombinantly produced glycosylated polypeptide, in particular a recombinantly produced glycosylated lubricin glycoprotein, wherein the method: (i) comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC); and (ii) further comprises a step of preparing a formulation comprising the recovered recombinantly produced polypeptide. In some embodiments, a method of reducing contaminants (e.g., polynucleotide and/or host cell protein contamination) in a formulation comprising a recombinantly produced glycosylated polypeptide described herein, further comprises one or more steps of depth filtration. In some embodiments, a step of depth filtration is performed prior to HIC. In some embodiments, depth filtration is performed after HIC. In some embodiments, depth filtration is performed prior to HIC and after HIC. In some embodiments, depth filtration is performed using a suitable filter, for example, a cellulose or polypropylene fiber-based filter, for example, a positively charged triple layer B1HC filter.

In a specific embodiment, the current invention relates to a method of making a pharmaceutical composition comprising a recombinantly produced glycosylated polypeptide, in particular, a recombinantly produced glycosylated lubricin glycoprotein, wherein the method: (i) comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC); and (ii) further comprises a step of preparing a pharmaceutical composition comprising the recovered recombinantly produced polypeptide. In some embodiments, the method of making a pharmaceutical composition comprising a recombinantly produced glycosylated polypeptide further comprises one or more steps of depth filtration. In some embodiments, a step of depth filtration is performed prior to HIC. In some embodiments, depth filtration is performed after HIC. In some embodiments, depth filtration is performed prior to HIC and after HIC. In some embodiments, depth filtration is performed using a suitable filter, for example, a cellulose or polypropylene fiber-based filter, for example, a positively charged triple layer B1HC filter.

In a further aspect, the present invention relates to a recombinantly produced glycosylated polypeptide purified by a method of the invention. In a specific embodiment, the present invention relates to lubricin purified by a method of the invention.

In some embodiments, the present invention relates to a glycosylated polypeptide produced by a method of the invention. In some embodiments, the invention relates to lubricin produced by a method of the invention.

In another aspect, the present invention relates to a recombinant glycosylated polypeptide, e.g., recombinant human lubricin, obtained by a method described herein. In some embodiments, the present invention relates to a recombinant glycosylated polypeptide that comprises amino acids 25-1404 of proteoglycan 4 isoform A (NCBI Reference Sequence: NP_005798.3; SEQ ID NO:1), amino acids 25-1404 of proteoglycan 4 isoform CRA_a (NCBI Reference Sequence: EAW91201.1; SEQ ID NO:2), or fragments and variants of a recombinant polypeptide comprising glycosylated repeats of the sequence KEPAPTT (SEQ ID NO:3), obtained by a method described herein.

In another aspect, the present invention relates to a pharmaceutical composition comprising a recombinantly produced glycosylated polypeptide, e.g., lubricin, purified by a method of the invention. In some embodiments, the present invention relates to a pharmaceutical composition comprising a protein that comprises amino acids 25-1404 of proteoglycan 4 isoform A (NCBI Reference Sequence: NP_005798.3; SEQ ID NO:1) or isoform CRA_a (NCBI Reference Sequence: EAW91201.1; SEQ ID NO:2). In some embodiments, a pharmaceutical composition described herein comprises a recombinantly produced glycosylated polypeptide (lubricin) and a pharmaceutical excipient or buffer (for example, a buffer comprising sodium phosphate, sodium chloride, and polysorbate (for example polysorbate 20)). In some embodiments, a pharmaceutical composition described herein is suitable for administration to a subject, for example, a subject in need of treatment (for example, treatment of an ocular surface disorder, for example, dry eye disease). In some embodiments, a pharmaceutical composition described herein is suitable for topical administration. In some embodiments, the subject in need of treatment is a primate. In some embodiments, the subject in need of treatment is a human.

In some embodiments, a pharmaceutical composition comprising a recombinantly produced glycosylated polypeptide purified by a method of the invention has a specified level or specified levels of the glycosylated polypeptide, contaminants, and/or purity. For example, in some embodiments, the purity of a pharmaceutical composition purified by a method of the invention is 80% or greater, 85% or greater, 90% or greater, 95% or greater, 96% or greater, 97% or greater, 98% or greater, 99% or greater, 99.5% or greater, 99.6% or greater, 99.7% or greater, 99.8% or greater, or 99.9% or greater, as determined by reversed phase chromatography (RPC). In some embodiments, a pharmaceutical composition comprising a recombinantly produced glycosylated polypeptide purified by a method of the invention comprises about 10% or less, about 5% or less, about 4% or less, about 3% or less, about 2% or less, or about 1% or less, preferably less than 1% of aggregates of the recombinantly produced glycosylated polypeptide, as determined by size exclusion chromatography (SEC). In some embodiments, a pharmaceutical composition comprising a recombinantly produced glycosylated polypeptide purified by a method of the invention comprises about 10% or less, about 5% or less, about 4% or less, about 3% or less, about 2% or less, or about 1% or less, preferably less than 1% of fragments of the recombinantly produced glycosylated polypeptide, as determined by size exclusion chromatography (SEC).

In some embodiments, a pharmaceutical composition comprising a recombinantly produced glycosylated polypeptide purified by a method of the invention comprises contaminants at a concentration of between 10 parts per million (ppm) and 10,000 ppm, between 10 ppm and 100 ppm, not more than about 10 ppm, or not more than about 5 ppm. In some embodiments, a pharmaceutical composition comprising a recombinantly produced glycosylated polypeptide purified by a method of the invention comprises≤1,000 ng/mg, ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg host cell protein content (e.g., ≤1,000 ng host cell protein/mg drug substance), as measured, for example, by ELISA. In some embodiments, a pharmaceutical composition comprising a recombinantly produced glycosylated polypeptide purified by a method of the invention comprises ≤200,000 pg/mg, ≤100,000 pg/mg, ≤50,000 pg/mg, ≤10,000 pg/mg, ≤5,000 pg/mg, ≤1,000 pg/mg, ≤500 pg/mg, ≤100 pg/mg, ≤50 pg/mg, ≤10 pg/mg, or ≤5 pg/mg residual host cell DNA content (e.g., ≤100,000 pg residual host cell DNA/mg drug substance), as measured by qPCR. In some embodiments, a pharmaceutical composition comprising a recombinantly produced glycosylated polypeptide purified by a method of the invention comprises a bacterial endotoxin content of less than 8 endotoxin units (EU)/mL, less than 1 EU/mL, less than 0.1 EU/mL, or less than 0.01 EU/mL, as determined by a bacterial endotoxin test (BET). In some embodiments, a pharmaceutical composition comprising a recombinantly produced glycosylated polypeptide purified by a method of the invention comprises a total aerobic microbial content (TAMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml. In some embodiments, a pharmaceutical composition comprising a recombinantly produced glycosylated polypeptide purified by a method of the invention comprises a total yeast and mold content (TYMC) of less than 1 CFU/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

In some embodiments, a pharmaceutical composition comprising a recombinantly produced glycosylated polypeptide purified by a method of the invention is stable at about 5° C. or lower for about 20, 21, 22, 23, 24, 25, 26, 27, 28, 29, 30, months or longer, from at least 20 to 30 months, or for at least 24 months. In some embodiments, a pharmaceutical composition comprising a recombinantly produced glycosylated polypeptide purified by a method of the invention is stable at 25° C. for 1 week, 2 weeks, 3 weeks, 4 weeks, 1 month, or longer, or from at least 1 week to 1 month, or for at least 1 month. In some embodiments, the stable composition of rhLubricin has an initial concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, or between about 0.15 mg/ml and about 0.45 mg/ml.

In some embodiments of the invention, a pharmaceutical composition comprising a recombinantly produced glycosylated polypeptide (for example, a recombinantly produced glycosylated polypeptide produced or purified by a method of the invention) has a concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 4.0 mg/ml, about 5.0 mg/ml, between about 1.0 mg/ml and 3.0 mg/ml, between about 1.6 mg/ml and 2.4 mg/ml, or between about 0.15 mg/ml and about 0.45 mg/ml. In some embodiments of the invention, a method of producing or purifying a highly glycosylated polypeptide (for example, lubricin) comprises a step of diluting the concentration of the highly glycosylated polypeptide. For example, in some embodiments, a method described herein comprises a step of diluting the concentration of the highly glycosylated polypeptide following a final chromatography step. In some embodiments, a method described herein comprises a step of diluting the concentration of the highly glycosylated polypeptide from a concentration of about 1.0 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 4.0 mg/ml, about 5.0 mg/ml, between about 1.0 mg/ml and 3.0 mg/ml, or between about 1.6 mg/ml and 2.4 mg/ml, to a concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, or between about 0.15 mg/ml and about 0.45 mg/ml. In some embodiments, a method described herein comprises a step of diluting the concentration of the highly glycosylated polypeptide in a suitable buffer (for example, a buffer comprising sodium phosphate, sodium chloride, and polysorbate (for example polysorbate 20)).

In some embodiments, a pharmaceutical composition comprising a recombinantly produced glycosylated polypeptide purified by a method of the invention is formulated in a final buffer solution comprising sodium phosphate, sodium chloride, and/or polysorbate (for example polysorbate 20). For example, in some embodiments, an rhLubricin composition described herein is formulated in a final buffer solution comprising 10 mM sodium phosphate, 140 mM sodium chloride, and 0.02% (w/v) polysorbate 20. In some embodiments, the final buffer solution has a pH of about 6.8, 6.9, 7.0, 7.1, or 7.2, between about 6.8 and about 7.2, between about 6.9 and about 7.1, or between about 6.9 and about 7.0.

Non-limiting embodiments of the present disclosure are described in the following list of embodiments:

1. A method of purifying a recombinant glycoprotein (e.g., recombinant lubricin), comprising the steps of subjecting a cell culture harvest containing said glcyoprotein to: a multimodal cation exchange chromatography (MCC), a multimodal anion exchange chromatography (MAC), and a hydrophobic interaction chromatography (HIC), which are performed in any order.

2. The method of embodiment 1, wherein the steps are performed in the following order: a) MCC, b) MAC, and c) HIC.

3. The method of embodiment 2, wherein prior to step a) the cell culture harvest is contacted with MgCl₂ and an endonuclease.

4. The method of embodiment 3, wherein the endonuclease is Benzonase® endonuclease.

5. The method of embodiment 3 or 4, wherein the cell culture harvest is cooled to 2-8° C. before being contacted with MgCl₂ and the endonuclease.

6. The method of any one of the preceding embodiments, further comprising a step of virus inactivation after the multimodal anion exchange chromatography (MAC) step and before the hydrophobic interaction chromatography (HIC) step.

7. The method of embodiment 6, wherein the virus inactivation step comprises adjusting the pH of the solution obtained from step b) to about 3.5.

8. The method of embodiment 7, wherein after incubating the solution for at least one hour, the pH is adjusted to about 7.0 before the hydrophobic interaction chromatography (HIC) step.

9. The method of any one of the preceding embodiments, comprising a virus removal step after the hydrophobic interaction chromatography (HIC) step.

10. The method of embodiment 9, further comprising an ultrafiltration step after the virus removal step.

11. The method of embodiment 9, comprising a virus inactivation step after the virus removal step.

12. The method of embodiment 11, wherein the virus inactivation step comprises adding a dimethylurea solution.

13. The method of embodiment 11 or 12, comprising an ultrafiltration step after the virus inactivation step.

14. The method of embodiment 1 or 2, further comprising one or more ultrafiltration and/or nanofiltration steps.

15. The method of embodiment 1 or 2, further comprising one or more virus inactivation steps.

16. The method of embodiment 1 or 2, further comprising one or more virus removal steps.

17. The method of any one of the preceding embodiments, wherein the recombinant lubricin glycoprotein comprises amino acid residues 25-1404 of SEQ ID NO:1 or SEQ ID NO:2.

18. The method of any one of the preceding embodiments, wherein at least 35% of the weight of the recombinant lubricin glycoprotein is from glycosidic residues.

19. The method of any one of the preceding embodiments, wherein at least 85%, at least 90%, or at least 95% of glycosylation of the lubricin glycoprotein is core 1 glycosylation.

20. A composition or formulation comprising a recombinant lubricin glycoprotein obtained by the method according to any one of the preceding embodiments.

21. A pharmaceutical composition comprising the recombinant lubricin glycoprotein according to embodiment 20 and a pharmaceutically acceptable excipient.

22. A method for treating an ocular surface disorder comprising a step of administering the recombinant lubricin glycoprotein according to embodiment 21 to a patient.

23. A method of producing a recombinant lubricin glycoprotein comprising the steps of a) generating a Chinese Hamster Ovary (CHO) cell clone which produces the recombinant lubricin glycoprotein, b) cultivating the CHO host cell under suitable conditions, thereby obtaining a cell culture containing a recombinant lubricin glycoprotein, and c) purifying the recombinant lubricin glycoprotein from the cell culture according to the method of any one of embodiments 1 to 19.

24. A method of producing a recombinant lubricin glycoprotein comprising the steps of a) cultivating under suitable conditions mammalian host cells that comprise a nucleic acid molecule that encodes a lubricin protein, and b) purifying the recombinant lubricin glycoprotein from the cell culture according to the method of any one of embodiments 1 to 19.

25. The method of embodiment 24, wherein the mammalian host cells are Chinese Hamster Ovary Cells.

26. The method of embodiment 25, wherein the CHO cells are CHO-M cells.

27. The method of embodiment 20, wherein the lubricin comprises less than 1 percent dimers and related substances of higher molecular mass, less than 10 ppm generic host cell protein (HCP), less than 0.006 pg/IU FSH DNA, and having a purity of more than 97 percent.

Specific preferred embodiments of the invention will become evident from the following more detailed description of certain preferred embodiments and the claims.

### BRIEF DESCRIPTION OF THE DRAWINGS

FIG. 1 shows a flow diagram and description of the present invention purification process.

FIG. 2 shows a flow chart of a particular embodiment of the invention.

FIG. 3 shows a flow chart of a particular embodiment of the invention.

FIG. 4 is an overlay of SEC chromatograms for an initial drug substance (DS) batch and a clinical drug substance batch. The insert is a zoom displaying a minor peak observed for the clinical drug substance batch.

FIG. 5 is an overlay of SEC chromatograms for an initial drug substance (DS) batch and a clinical drug substance batch, for detected aggregates.

FIG. 6 is an overlay of RPC chromatograms for an initial drug substance (DS) batch and a clinical drug substance batch.

FIG. 7 shows SV-AUC absorbance profiles at 230 nm for an initial drug substance (DS) batch and a clinical drug substance batch, measured in triplicate.

FIG. 8 is a comparison chromatogram of Molecular Weight Marker Solution (MWM solution).

FIG. 9 is another comparison chromatogram of Molecular Weight Marker Solution (MWM solution).

FIG. 10 is a chromatogram demonstrating calculation of resolution.

FIG. 11 is an example chromatogram of limit of qualification (LOQ) solution signal-to-noise ratio for lubricin peak.

FIG. 12 is an example chromatogram (full view) of lubricin.

FIG. 13 is an example chromatogram showing main peak with aggregate and fragment and solvent peaks.

FIG. 14 is an example chromatogram illustrating the method for calculating tailing factor.

FIG. 15 is an example chromatogram of a reference solution.

FIG. 16 15 is an example chromatogram of a reference solution.

FIG. 17 is an example chromatogram of drug substance stressed two weeks at 40 degrees and high pH.

FIG. 18 is an example chromatogram I of MWM solution.

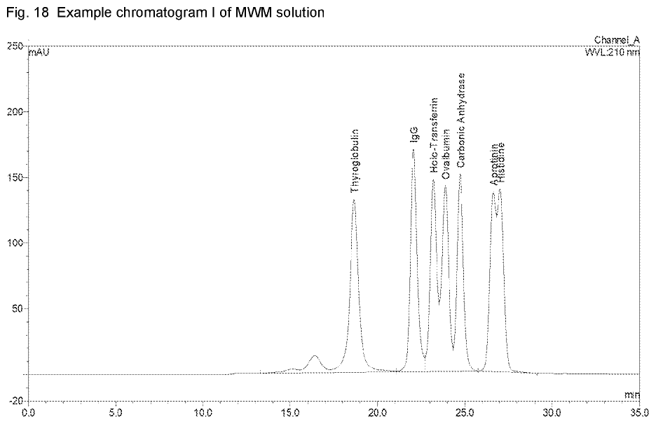

FIG. 19 is an example chromatogram II of MWM solution.

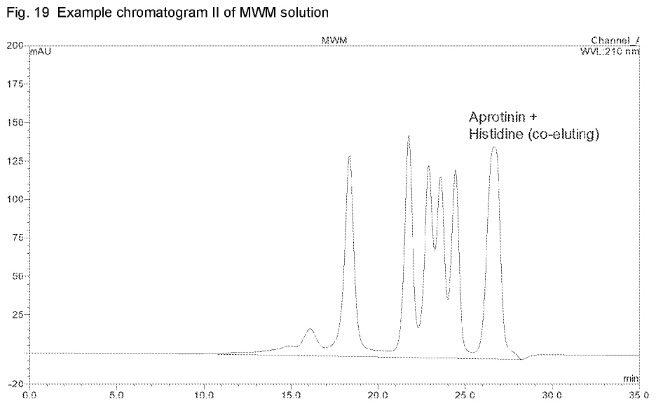

FIG. 20 is a chromatogram demonstrating resolution calculation.

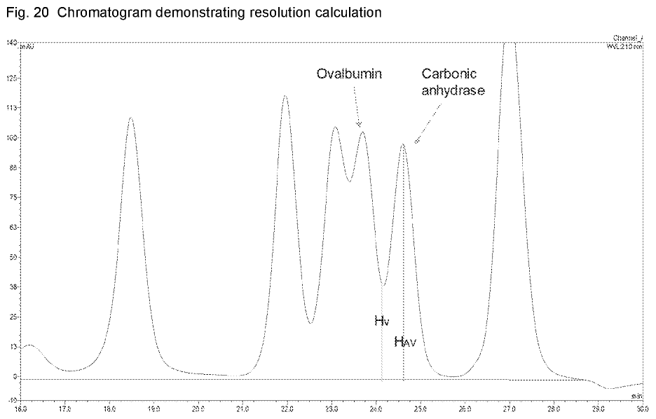

FIG. 21 is a close up chromatograph with a solvent peak and a blank.

FIG. 22 is a chromatogram demonstrating integration procedure.

FIG. 23 is a close-up and description of a main double-peak.

FIG. 24 close up chromatograph of a main peak.

FIG. 25 is an example chromatogram of stressed drug substance batch of lubricin (40 degrees, 2 days).

FIG. 26 is a table showing O-glycans detected and identified in two drug substance batches.

FIG. 27 is an overlay of MALDI TOF spectra (mirrored view) of N-glycans of two drug substance batches.

FIG. 28A and 28B are examples of reference solution chromatograms.

FIG. 29 is a chromatogram showing the early-eluting peaks (EP), main peaks (MP), and late-eluting peaks (LP).

### DETAILED DESCRIPTION

Unless defined otherwise, all technical and scientific terms used herein have the same meaning as commonly understood by those of ordinary skill in the art to which the present disclosure pertains.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by 20 percent or in some instances 10 percent, or in some instances 5 percent, or in some instances 1 percent, or in some instances 0.1 percent from the specified value, as such variations are appropriate to perform the disclosed methods. It is to be understood, although not always explicitly stated that all numerical designations are preceded by the term “about”.

The term “about” when referring to a measurable value such as an amount, a temporal duration, and the like, is meant to encompass variations of (+) or (−) 20 percent, or in some instances (+) or (−) 10 percent, or in some instances (+) or (−) 5 percent, or in some instances (+) or (−) 1 percent, or in some instances (+) or (−) 0.1 percent from the specified value, as such variations are appropriate to perform the disclosed methods.

It also is to be understood, although not always explicitly stated, that the reagents described herein are merely examples and that equivalents of such are known in the art.

The term “eluate” as used herein refers to a solution obtained by elution. Thus, a solution obtained from a chromatography step after washing with a wash buffer is an eluate.

The terms “peptide”, “polypeptide”, and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

As used herein, the term “glycosylated” is defined as a saccharide (or sugar) covalently attached, i.e. linked, to an amino acid. Specifically, the saccharide is linked to the side-chain of the amino acid. The terms “glycosylated peptide”, “glycosylated polypeptide”, and “glycosylated protein” are used interchangeably herein, and refer to a polypeptide that has post-translational modifications in the form of glycosylation. Glycosylation is well known to those of skill in the art, and includes all types of glycosylation. In certain embodiments, the methods of the invention are particularly useful for purifying proteins that have more O-glycosylation than N-glycosylation. In certain embodiments, the O-glycosylation is predominantly Core 1 subtype O-glycans (e.g., more Core 1 than Core 2 or Core 3 or Core 4 O-glycans). In other embodiments, the Core 1 glycosylation of a protein being produced or purified by a method of the present invention is at least about 85% of the total glycosylation (e.g., at least about 85%, 86%, 87%, 88%, 89%, 90%, 91%, 92%, 93%, 94%, 95%, 96%, 97%, 98%, 99%, or more). In some embodiments, Core 1 glycosylation comprises about 85%, about 88%, about 89%, about 90%, about 91%, about 92%, about 93%, about 94%, about 95%, greater than about 85%, greater than about 88%, greater than about 89%, greater than about 90%, greater than about 91%, greater than about 92%, greater than about 93%, greater than about 94%, or greater than about 95% of O-glycosylation of a protein produced or purified by a method of the present invention. Core 1 and other O-glycan subtypes involved in O-glycosylation are described for example in Chapter 8, O-Glycans, Essentials of Glycobiology, 3^{rd} Ed, 1999, Consortium of Glycobiology Editors, La Jolla, Calif. Glycosylation can be determined as described in the Examples herein.

In some embodiments, O-glycosylation of a glycosylated protein produced or purified by a method described herein comprises sialic acid-based and monosaccharide-based O-glycan species. For example, O-glycosylation of a glycosylated protein purified by a method described herein can include the following Core 1 glycan species: galactose-β-1,3-N-acetylgalactosamine (also known as Galβ1-3GalNAc, galactose-N-acetylated galactose, Gal-GalNAc, or Core 1), monosialylated Gal-GalNAc (also known as N-acetylneuraminic acid α 2,3-galactose beta 1,3-N-acetylgalactosamine, Neu5Acα2-3Galβ1-3GalNAc, or 2,3-NeuAc Core 1), disialylated Gal-GalNAc (also known as N-acetylneuraminic acid α 2,3-galactose β 1,3-(N-acetylneuraminic acid alpha 2,6-)N-acetylgalctosamine, Neu5Acα2-3(Neu5Acα2-6)Galβ(1-3GalNAc, or 2*NeuAc Core 1), and N-glycolyneuraminic acid (N-Glycolylneuraminic acid α 2,3-galactose β 1,3-N-acetylgalactosamine, Neu5Gcα2-3Galβ(1-3GalNAc, 2,3-NeuGc Core 1, or NGNA).

For example, in some embodiments, the O-glycan composition of a glycosylated protein produced or purified by a method described herein comprises about 5% to about 10%, about 6% to about 10%, about 7% to about 10%, about 7% to about 12%, or about 7% to about 9% Gal-GalNAc. In some embodiments, the O-glycan composition of a glycosylated protein produced or purified by a method described herein comprises about 70% to about 80%, about 70% to about 90%, about 75% to about 95%, about 75% to about 90%, about 75% to about 85%, or about 75% to about 80% 2,3-NeuAc Core 1. In some embodiments, the O-glycan composition of a glycosylated protein produced or purified by a method described herein comprises about 1%, about 2%, about 3%, about 4%, about 5%, about 6%, about 7%, about 1% to about 6%, about 2% to about 5%, about 3% to about 6%, about 3% to about 5%, or about 2% to about 4% 2*NeuAc Core 1. In some embodiments, the O-glycan composition of a glycosylated protein produced or purified by a method described herein comprises about 1%, about 2%, about 3%, about 4%, about 5%, about 1% to about 5%, about 2% to about 5%, about 3% to about 5%, about 1% to about 2%, or about 1% to about 3% NGNA. In some embodiments, the O-glycan composition of a glycosylated protein produced or purified by a method described herein comprises about 5% to about 10% Gal-GalNAc, about 75% to about 85% 2,3-NeuAc Core 1, about 1% to about 5% 2*NeuAc Core 1, and about 1% to about 2% NGNA. In some embodiments, the O-glycan composition of a glycosylated protein produced or purified by a method described herein comprises about 7% Gal-GalNAc, about 80% 2,3-NeuAc Core 1, about 3% 2*NeuAc Core 1, and about 1% NGNA. In some embodiments, the O-glycan composition of a glycosylated protein produced or purified by a method described herein comprises about 7% or more Gal-GalNAc, about 80% or more 2,3-NeuAc Core 1, about 3% or more 2*NeuAc Core 1, and about 1% or more NGNA. In some embodiments, the O-glycan composition of a glycosylated protein produced or purified by a method described herein comprises at least 7% Gal-GalNAc, at least 80% 2,3-NeuAc Core 1, at least 3% 2*NeuAc Core 1, and at least 1% NGNA.

In some embodiments, the O-glycan composition of a glycosylated protein produced or purified by a method described herein comprises about 9% Gal-GalNAc, about 76% 2,3-NeuAc Core 1, about 3% 2*NeuAc Core 1, and about 1% NGNA. In some embodiments, the O-glycan composition of a glycosylated protein produced or purified by a method described herein comprises about 9% or more Gal-GalNAc, about 76% or more 2,3-NeuAc Core 1, about 3% or more 2*NeuAc Core 1, and about 1% or more NGNA. In some embodiments, the O-glycan composition of a glycosylated protein produced or purified by a method described herein comprises at least 9% Gal-GalNAc, at least 76% 2,3-NeuAc Core 1, at least 3% 2*NeuAc Core 1, and at least 1% NGNA. In some embodiments, the O-glycan composition of a glycosylated protein produced or purified by a method described herein comprises at least 7% Gal-GalNAc, at least 76% 2,3-NeuAc Core 1, at least 3% 2*NeuAc Core 1, and at least 1% NGNA.

In some embodiments, the O-glycan composition of a glycosylated protein produced or purified by a method described herein comprises about 9% Gal-GalNAc, about 76% 2,3-NeuAc Core 1, about 3% 2*NeuAc Core 1, about 1% NGNA, and about 11% of a NANA related glycan (for example, an oxidized form of monosialylated (NANA) Gal-GalNAc). In some embodiments, the O-glycan composition of a glycosylated protein produced or purified by a method described herein comprises about 7% Gal-GalNAc, about 80% 2,3-NeuAc Core 1, about 3% 2*NeuAc Core 1, about 1% NGNA, and about 8% of a NANA related glycan (for example, an oxidized form of monosialylated (NANA) Gal-GalNAc).

In some embodiments, the percentage of each O-glycan (for example, Gal-GalNAc, 2,3-NeuAc Core 1, 2*NeuAc Core 1, or NGNA) is calculated as the percentage of the sum of the following O-glycans: Gal-GalNAc, 2,3-NeuAc Core 1, 2*NeuAc Core 1, and NGNA. In some embodiments, the percentage of each O-glycan is calculated as the percentage of the sum of the following: Gal-GalNAc, 2,3-NeuAc Core 1, 2*NeuAc Core 1, NGNA, and a NANA-related glycan (for example, an oxidized form of 2,3-NeuAc Core 1).

In some embodiments, a highly glycosylated protein produced or purified by a method of the invention is characterized by the content of glycosylation species. Gylcoyslation species content can be determined using any suitable method, for example, ion chromatography (separation mechanism: anion exchange)/pulsed amperometric detection, which is based on the general method of USP-NF <1065>, “Ion Chromatography.” The analytical method can be used for quantification of sialic acid glycan species, including N-acetylneuraminic acid (NANA) and N-glycolylneuraminic acid (NGNA) of a purified glycosylated protein after acidic release by ion chromatography (IC). Additionally, the analytical method can be used for the quantification of monosaccharide-containing glycan species, for example, the monosaccharides D-(+)-Galactose (Gal) and N-Acetyl-D-galactosamine (GalNAc), after acidic release by IC. In some embodiments, the sialic acid glycan content of a glycosylated protein produced or purified by a method described herein comprises about 50 μg or more NANA per mg of glycosylated protein and about 10 μg or less NGNA per mg of glycosylated protein. In some embodiments, the monosacchande glycan content of a glycosylated protein produced or purified by a method described herein comprises about 100 μg or more Gal per mg of glycosylated protein and about 100 μg or more GalNAc per mg of glycosylated protein.

In some embodiments, a glycosylated protein produced or purified by a method described herein comprises one or more N-glycosylation species, for example, one or more mannosylated glycans, for example, mannose-5 glycan (also known as Man-5 N-linked oligosaccharide and oligomannose 5 glycan; “Man-5”), mannose-6 glycan (also known as Man-6 N-linked oligosaccharide and oligomannose 6 glycan; “Man-6”), and mannose-7 glycan (also known as Man-7 N-linked oligosaccharide and oligomannose 7 glycan; “Man-7”). N-glycan species are bound to an amide nitrogen of an asparagine (Asn) residue of a protein. Described herein is a recombinant lubricin protein of SEQ ID NO:1 or 2 (for example, a recombinant lubricin protein of SEQ ID NO:1 or 2 produced or purified using a method described herein) or a recombinant lubricin protein comprising amino acid residues 25-1404 of SEQ ID NO:1 or SEQ ID NO:2, wherein asparagine 1135 of SEQ ID NO:1 or 2 is N-glycosylated. Also described herein is a composition comprising a recombinant lubricin protein (for example, a recombinant lubricin protein produced or purified using a method described herein) of SEQ ID NO:1 or 2 or comprising amino acid residues 25-1404 of SEQ ID NO:1 or SEQ ID NO:2, wherein asparagine 1135 of SEQ ID NO:1 or 2 is N-glycosylated. Also described herein is a method of purifying a recombinant lubricin protein of SEQ ID NO:1 or 2 or a recombinant lubricin protein comprising amino acid residues 25-1404 of SEQ ID NO:1 or SEQ ID NO:2, wherein asparagine 1135 of SEQ ID NO:1 or 2 is N-glycosylated. Also described herein is a method of formulating a composition comprising recombinant lubricin of SEQ ID NO:1 or 2 (for example, recombinant lubricin of SEQ ID NO:1 or 2 purified using a method described herein) or a recombinant lubricin protein comprising amino acid residues 25-1404 of SEQ ID NO:1 or SEQ ID NO:2, wherein the recombinant lubricin is N-glycosylated.

In some embodiments described herein, N-glycosylation of asparagine 1135 of a recombinant lubricin of SEQ ID NO:1 or 2 is Man-5, Man-6, or Man-7. Thus, in some embodiments, a recombinant lubricin protein (for example, a recombinant lubricin protein produced or purified using a method described herein) of SEQ Ill NO:1 or 2 or a recombinant lubricin protein comprising amino acid residues 25-1404 of SEQ ID NO:1 or SEQ ID NO:2 comprises N-glycosylated asparagine 1135 of the recombinant lubricin of SEQ ID NO:1 or 2, and the N-glycosylation is Man-5, Man-6, or Man-7. In some embodiments, described herein is a composition comprising recombinant lubricin (for example, recombinant lubricin produced or purified using a method described herein), wherein N-glycosylation of asparagine 1135 of the recombinant lubricin of SEQ ID NO:1 or 2 is Man-5, Man-6, or Man-7. In some embodiments, a composition comprising recombinant lubricin of SEQ ID NO:1 or 2 (for example, recombinant lubricin of SEQ ID NO:1 or 2 produced or purified using a method described herein) or a recombinant lubricin protein comprising amino acid residues 25-1404 of SEQ ID NO:1 or SEQ ID NO:2, comprises N-glycosylation of asparagine 1135 of the recombinant lubricin of SEQ ID NO:1 or 2, wherein the N-glycosylation is Man-5, Man-6, Man-7, or a mixture thereof (for example: Man-5, Man-6, and Man-7; Man-5 and Man-6; Man-5 and Man-7; Man-6 and Man-7; Man-5; Man-6; or Man-7). In some embodiments, a composition described herein comprises a plurality of recombinant lubricin polypeptides, wherein a portion of the recombinant lubricin proteins are N-glycosylated. For example, in some embodiments, a composition described herein comprises a plurality of recombinant lubricin polypeptides comprising amino acid residues 25-1404 of SEQ ID NO:1 or SEQ ID NO:2, that share the same polypeptide sequence, but which are differentially N-glycosylated (for example, a portion of the polypeptides comprise an N-glycosylated asparagine 1135 of the recombinant lubricin of SEQ ID NO:1 or 2, and N-glycosylation of asparagine 1135 is Man-5, Man-6, Man-7, or a mixture thereof (for example: Man-5, Man-6, and Man-7; Man-5 and Man-6; Man-5 and Man-7; Man-6 and Man-7; Man-5; Man-6; or Man-7)).

In some embodiments, a “glycosylated” polypeptide purified by a method of the invention is heavily glycosylated. As used herein, a “heavily glycosylated” polypeptide comprises at least 25% glycosylation by weight (e.g., at least 25%, 26%, 27%, 28%, 29%, 30%, 31%, 32%, 33%, 34%, or 35% by weight, or higher), for example as determined by analytical ultracentrifugation (AUC) using sedimentation equilibrium (SE-AUC) and sedimentation velocity (SV-AUC) modes. For example, recombinant lubricin produced in a mammalian host cell (e.g., a Chinese Hamster Ovary cell) as described in published U.S. Patent Application Number US20160304572 is a heavily glycosylated polypeptide. Mucins and mucin-like proteins are also considered highly glycosylated proteins (Debauilleul et al., 1998, J. Biol. Chem. 273:881-890; Gendler et al., 1995, Annu. Rev. Physiol. 57:607-634; van Klinken et al.,. 1998, Glycobiology 8:67-75). In certain embodiments, the methods provided herein are useful for purifying glycosylated proteins with at least 25%, 26%, 27%, 28%, 29%, 30%, 35%, 40%, 45%, 50%, or more glycosylation by weight (e.g., at least 30% of the molecular weight of the protein is from the glycosidic residues), including mucin proteins and mucin-like proteins such as lubricin.

The term “recombinant”, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant polypeptide” or “recombinantly produced polypeptide” refers to a polypeptide produced by expression from a recombinant polynucleotide. The term “recombinant”, as used with respect to a host cell or a virus, refers to a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polynucleotide”, “oligonucleotide”, “nucleic acid” and “nucleic acid molecule” are used interchangeably herein to include a polymeric form of nucleotides, either ribonucleotides or deoxyribonucleotides. This term refers only to the primary structure of the molecule. Thus, the terms include triple-, double- and single-stranded DNA, as well as triple-, double- and single-stranded RNA. The terms also include such molecules with modifications, such as by methylation and/or by capping, and unmodified forms of a polynucleotide. More particularly, the terms “polynucleotide”, “oligonucleotide”, “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non-nucleotidic backbones, polymers, and other synthetic sequence-specific nucleic acid polymers providing that the polymers contain nucleobases in a configuration which allows for base pairing and base stacking, such as is found in DNA and RNA.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50 percent homologous; if 90 percent of the positions (e.g., 9 of 10), are matched or homologous, the two sequences are 90 percent homologous.

A “host cell”, as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a recombinant polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell may be stably or transiently introduced into the cell. In some embodiments, recombinant lubricin is produced in a mammalian host cell, such as a Chinese Hamster Ovary (CHO) cell. In another embodiment, the CHO cells are CHO-M cells as described in U.S. Patent Application Publication No. 2016/304572, the disclosure of which is incorporated herein by reference (see also Girod et al., Nat. Methods 4(9):747-53 (2007), and U.S. Pat. Nos. 7,129,062 and 8,252,917 and U.S. Patent Application Publication Nos. 2011/0061117; 2012/0231449; and 2013/0143264, the disclosures of which are incorporated herein by reference).

The terms “purifying”, “isolating”, and the like, refer to the removal of a desired substance, e.g., a recombinant protein, from a solution containing undesired substances, e.g., contaminates, e.g., polynucleotides, host cell protein, or the removal of undesired substances from a solution containing a desired substance, leaving behind essentially only the desired substance. In some instances, a purified substance may be essentially free of other contaminants, e.g., polynucleotides, e.g., host cell proteins. Purifying, as used herein, may refer to a range of different resultant purities, e.g., wherein the purified substance makes up more than 80 percent of all the substance in the solution, including more than 85 percent, more than 90 percent, more than 91 percent, more than 92 percent, more than 93 percent, more than 94 percent, more than 95 percent, more than 96 percent, more than 97 percent, more than 98 percent, more than 99 percent, more than 99.5 percent, more than 99.9 percent, and the like. As will be understood by those of skill in the art, generally, components of the solution itself, e.g., water or buffer, or salts are not considered when determining the purity of a substance.

The terms “contaminant” and “impurity” refer to undesired substance, e.g., polynucleotides (e.g., DNA and/or RNA) or proteins (e.g., host cell proteins), that are present in a solution or in a drug product that contains the protein being purified. Contaminants include, for example, host cell proteins from cells used to recombinantly express the protein being purified, proteins that are part of an absorbent used in an affinity chromatography step that may leach into a sample during prior affinity chromatography step, and mis-folded variants of the target protein itself. In certain embodiments, contaminants that remain in a sample during the purification process are referred to as “residual” (e.g., “residual DNA”). Aggregates and degradants of the target protein are also considered contaminants.

The term “degradant” as used herein includes fragments (i.e., peptide fragments) of a recombinant target protein caused by degradation. Degradation products can be measured using techniques well known to those of skill in the art, and analytical results can be provided for drug substance and drug product batches for clinical, safety, and stability testing, as well as for batches representing commercial manufacturing processes.

The term “host cell proteins” (HCP) includes proteins encoded by the host cell comprising DNA encoding a target protein that is to be purified. Host cell proteins may be contaminants of the protein to be purified, the levels of which may be reduced by purification. Host cell proteins can be detected using assays well known to those of skill in the art, such as gel electrophoresis and staining and/or ELISA assay, and the like. Host cell proteins include, for example, Chinese Hamster Ovary (CHO) proteins (CHOP) produced during the expression of recombinant target proteins.

The logarithmic removal capacity of HCP can be calculated with the equation below. The HCP concentration in load and eluate can be determined in parts per million (ppm) by a multianalyte enzyme-linked immunosorbent assay (ELISA).

HCP log removal capacity=log₁₀(HCP in load [pm])−log₁₀(HCP in eluate [pm])

In certain embodiments, for patient safety the recommended upper limit for HCPs is 100 ppm HCPs (or <100 ng HCP per mg of therapeutic protein) in the final drug formulation (Champion, et al. 2005, BioProcess Int, 3:52; Chon & Zarbis-Papastoitsis, 2011, N Biotechnol. 28(5):458-63; Zhu-Shimoni, et al., 2014, Biotechnol Bioeng; 111:2367-79).

To prevent the unwanted effects of residual DNA, the FDA determined 10 pg of DNA per dose as an achievable analytical limit of sensitivity (Briggs and Panfili, 1991, Anal. Chem., 63:850-859).

The term “buffered” as used within this application denotes a solution in which changes of pH due to the addition or release of acidic or basic substances is leveled by a buffer substance. Any buffer substance resulting in such an effect can be used. Preferably pharmaceutically acceptable buffer substances are used, such as e.g. phosphoric acid or salts thereof, acetic acid or salts thereof, citric acid or salts thereof, morpholine or salts thereof, 2-(N-morpholino) ethanesulfonic acid or salts thereof, histidine or salts thereof, glycine or salts thereof, or Tris (hydroxymethyl) aminomethane (TRIS) or salts thereof. Especially preferred are phosphoric acid or salts thereof, or acetic acid or salts thereof, or citric acid or salts thereof, or histidine or salts thereof. Optionally, the buffered solution may comprise an additional salt, such as e.g. sodium chloride, sodium sulphate, potassium chloride, potassium sulfate, sodium citrate, or potassium citrate. Optionally, the buffered solution may comprise an additional component, for example, a polysorbate, for example, polysorbate 20.

As used herein, a protein is “recovered” or “separated” or “removed” when the concentration of the target protein is higher in the resulting product (e.g., drug product) than in the starting solution or mixture (e.g., cell culture harvest). In certain embodiments, recovered target protein can be expressed as a yield.

As used herein, “yield” is represented as the percentage of the residual content per volume in the eluate in comparison to the load content per volume before a purification step(s). A “yield” can be, for example, the amount of target protein (e.g., recombinant lubricin) in a sample (e.g., formulation or composition) that is present after a purification step compared with the amount that was present before that step was performed (e.g., the amount in the load compared with the eluate). The protein content of the load and eluate is measured, for example, by analytical size-exclusion chromatography (SEC).

The term “pharmaceutically acceptable carrier” or “pharmaceutically acceptable excipient” as used herein refers to solvents and other substances that are compatible with pharmaceutical administration. The use of such agents is well known in the art. Compositions described herein may also contain other active compounds providing supplemental, additional, or enhanced therapeutic functions.

The term “pharmaceutical composition” as used herein refers to a composition comprising at least one active ingredient (for example, recombinant human lubricin) as disclosed herein formulated together with one or more pharmaceutically acceptable excipients.

### Protein Purification Process

It is an object of the present invention to provide a purification process of recombinantly expressed glycosylated polypeptide, e.g., lubricin and its multimeric complexes, from contaminants, wherein the purification process enables efficient purification of said glycosylated polypeptide, e.g., lubricin, from impurities while retaining function, avoiding aggregation and maintaining high yield of the final product. The inventors have now found the purification method which is particularly suitable for purification of recombinantly expressed glycosylated polypeptides, e.g., lubricin, at a scale suitable for commercial pharmaceutical exploitation.

In the first aspect, the current invention relates to a method of purifying a recombinantly produced glycosylated polypeptide, in particular a recombinantly produced glycosylated lubricin protein, wherein the method comprises three chromatography steps: (a) a multimodal cation exchange chromatography (MCC) step; (b) a multimodal anion exchange chromatography (MAC) step; and (c) a hydrophobic interaction chromatography (HIC) step.

In some embodiments, the three chromatography steps may be carried out in any sequence. In a specific embodiment, the current invention relates to a method of purifying a recombinantly produced glycosylated polypeptide, in particular a recombinantly produced glycosylated lubricin protein, wherein the method comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC).

As used herein, the term “successive chromatography steps ” refers to steps that are carried out in the presented order, but may include other steps before the first recited step, between the recited steps, and/or after the last recited step.

In the second aspect, the current invention relates to a method of reducing polynucleotide and/or host cell protein contamination in a formulation comprising a recombinantly produced glycosylated polypeptide, in particular a recombinantly produced glycosylated lubricin, wherein the method:
(i) comprises three chromatography steps: (a) a multimodal cation exchange chromatography (MCC) step; (b) a multimodal anion exchange chromatography (MAC) step; and (c) a hydrophobic interaction chromatography (HIC) step; and
(ii) further comprises a step of preparing a formulation comprising the recovered recombinantly produced polypeptide.

In some embodiments, a method of reducing polynucleotide and/or host cell protein contamination in a formulation comprising a recombinantly produced glycosylated polypeptide described herein, further comprises a step of depth filtration. In some embodiments, the step of depth filtration is performed prior to the HIC step. In some embodiments, depth filtration is performed using a suitable filter, for example, a cellulose or polypropylene fiber-based filter, for example, a positively charged triple layer B1HC filter.

In some embodiments, the three chromatography steps may be carried out in any sequence. In a specific embodiment, the current invention relates to a method of reducing polynucleotide and/or host cell protein contamination in a formulation comprising a recombinantly produced glycosylated polypeptide, in particular a recombinantly produced glycosylated lubricin, wherein the method:
(i) comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC); and
(ii) further comprises a step of preparing a formulation comprising the recovered recombinantly produced polypeptide.

In some embodiments, a method of reducing polynucleotide and/or host cell protein contamination in a formulation comprising a recombinantly produced glycosylated polypeptide described herein, further comprises a step of depth filtration. In some embodiments, the step of depth filtration is performed prior to the HIC step. In some embodiments, depth filtration is performed using a suitable filter, for example, a cellulose or polypropylene fiber-based filter, for example, a positively charged triple layer B1HC filter.

In certain embodiments, the methods of the invention further comprise one or more viral inactivation (VIN) treatment steps and viral removal steps as described herein. Various methods of virus inactivation are known to those of skill in the art and can be used in a method of the invention, including but not limited to pasteurization, terminal dry heat, vapor heat, solvent/detergents, and acid pH. Virus removal procedures are also well known, including but not limited to precipitation, chromatography, and nanofiltration. Viral inactivation and removal can be done in-process (e.g., nanofiltration and solvent/detergent treatment, pasteurization, steam-treatment, and/or incubation at pH 4) or terminal in the final container (e.g., terminal pasteurization or terminal dry-heat treatment). In some embodiments, the VIN step comprises incubating a solution during a purification method of the invention with N,N-Dimethylurea (DMU). In some embodiments, the VIN step comprises incubating a solution during a purification step of the invention with a detergent, for example, Triton X-100. In some embodiments, the VIN step comprises incubating a solution during a purification step of the invention with 2% Triton X-100 reduced for 30 minutes, 40 minutes, 50 minutes, 60 minutes, 70 minutes, 80 minutes, 90 minutes, 100 minutes, 110 minutes, or 120 minutes. In an embodiment described herein, the VIN step comprises incubating a solution during a purification step of the invention with 2% Triton X-100 reduced for 60 minutes to 70 minutes.

In some embodiments, the disclosed methods comprise a step viral inactivation in an acidic pH solution (e.g., a solution of about pH 3, about pH 3.4, about pH 3.5, about pH 3.6, about pH 4, about pH 3.4 to about pH 3.6, or about pH 3 to about pH 4). In some embodiments, the step of viral inactivation in an acidic pH solution comprises adjusting the pH of an rhLubricin composition to pH 3.4-3.6, for example, adjusting the pH of an rhLubricin composition with a 0.5 M phosphoric acid solution. The step of viral inactivation in an acidic pH solution can further include incubating the rhLubricin composition at 17-25° C. for 60-90 min, and adjusting the pH of the rhLubricin composition to pH 7.0 with a solution, for example, a 1 M tris(hydroxymethyl)aminomethane (Tris) solution. The step of viral inactivation in an acidic pH solution can further include filtering the rhLubricin composition through a 0.2 μm filter. In some embodiments of a method described herein, a viral inactivation step, for example, by incubation in an acidic pH solution, is performed after a multimodal anion exchange chromatography (MAC) step and before a hydrophobic interaction chromatography (HIC) step.

In certain embodiments, a method of the present invention comprises inactivating any viruses in a solution after the MAC step, wherein the pH of the solution obtained from the MAC step is adjusted to about e.g., 4.0 or less, such as about 3.4, 3.5, 3.6, 3.7, 3.8, 3.9, or 4.0, and incubated for at least about 60 minutes, and then adjusted to a neutral pH (e.g., about 7.0).

In another embodiment, a method of the invention further comprises a virus removal filtration (VRF) and virus inactivation (VIN) treatment steps after the HIC step. The VRF step comprises, for example, a nanofilter as described herein. In some embodiments, the VIN step comprises incubating the solution from the VRF step with a detergent. In another embodiment, the VIN step comprises incubating the solution from the VRF step with N,N-Dimethylurea (DMU). In certain embodiments, the concentration of DMU is about 1 M , 2

M, 3 M, 4 M, or 5 M. In a preferred embodiment, the concentration of DMU is 3 M.

In one aspect, the current invention relates to a recombinant glycosylated polypeptide, e.g., recombinant human lubricin, obtained by a method comprising three chromatography steps:
(a) a multimodal cation exchange chromatography (MCC) step;
(b) a multimodal anion exchange chromatography (MAC) step; and
(c) a hydrophobic interaction chromatography (HIC) step.
   In some embodiments, the method further comprises a step of preparing a formulation comprising the recombinant glycosylated polypeptide. In some embodiments, the method further comprises a step of depth filtration. In some embodiments, the step of depth filtration is performed prior to the HIC step. In some embodiments, depth filtration is performed using a suitable filter, for example, a cellulose or polypropylene fiber-based filter, for example, a positively charged triple layer B1HC filter. In some embodiments, the recombinant glycosylated polypeptide obtained by a method described herein is recombinant human lubricin comprising amino acids 25-1404 of proteoglycan 4 isoform A (NCBI Reference Sequence: NP_005798.3; SEQ ID NO:1), recombinant human lubricin comprising amino acids 25-1404 of proteoglycan 4 isoform CRA_a (NCBI Reference Sequence: EAW91201.1; SEQ ID NO:2), or fragments and variants of a recombinant polypeptide comprising glycosylated repeats of the sequence KEPAPTT (SEQ ID NO:3).

Methods of the present invention are particularly suitable for purification of a recombinantly produced polypeptide that is highly glycosylated (e.g., comprises at least 30% by weight glycosidic residues). Methods of the present invention are particularly suitable for purification of a recombinantly produced glycosylated polypeptide comprising negative charge carriers, for example a recombinantly produced glycosylated polypeptide comprising negative charge carriers in the central domain thereof, in particular in a mucin domain. Thus, a method of the present invention is particularly suitable for purification of recombinantly produced glycosylated lubricin proteins.

Full length (non-truncated) human lubricin (proteoglycan 4, isoform A, NCBI Reference Sequence: NP_005798.3) monomer sequence (SEQ ID NO:1) comprises 1404 amino acids, or approximately 151 kDa in core protein. A variant lubricin (proteoglycan 4, isoform CRA_a, NCBI Reference Sequence: EAW91201.1) monomer sequence (SEQ ID NO:2) also has 1404 amino acids. As used herein, “lubricin” or “lubricin protein” or “PRG4” include lubricin isoform A and lubricin isoform CRA_a, and further include fragments and variants thereof comprising glycosylated repeats of the sequence KEPAPTT (SEQ ID NO:3) and having substantially the same activity as full-length and naturally occurring lubricin. “rhLubricin” as used herein refers to recombinant human lubricin, and includes any human lubricin produced recombinantly. In some embodiments described herein, a “glycosylated protein,” a “glycosylated polypeptide,” a “highly glycosylated protein,” a “highly glycosylated polypeptide,” a “heavily glycosylated protein,” a “heavily glycosylated polypeptide,” a “heavily glycosylated recombinant protein,” “a heavily glycosylated recombinant polypeptide,” a “ recombinantly produced glycosylated protein,” a “recombinantly produced glycosylated polypeptide,” “a recombinantly produced glycosylated glcyoprotein,” or a “recombinantly produced polypeptide that is highly glycosylated,” (including a “glycosylated protein,” a “glycosylated polypeptide,” a “highly glycosylated protein,” a “highly glycosylated polypeptide,” a “heavily glycosylated protein,” a “heavily glycosylated polypeptide,” a “heavily glycosylated recombinant protein,” “a heavily glycosylated recombinant polypeptide,” a “ recombinantly produced glycosylated protein,” a “recombinantly produced glycosylated polypeptide,” “a recombinantly produced glycosylated glcyoprotein,” or a “recombinantly produced polypeptide that is highly glycosylated” produced or purified using a method described herein) can be lubricin of SEQ ID NO:1, SEQ ID NO:2, or a mixture thereof, including fragments (for example, amino acid residues 25-1404 of SEQ ID NO:1 or SEQ ID NO:2) and variants thereof comprising glycosylated repeats of the sequence KEPAPTT (SEQ ID NO:3) and having substantially the same activity as full-length and naturally occurring lubricin.

In some embodiments, a glycosylated recombinant human lubricin produced or purified using a method described herein is characterized by a molecular weight of about 280 kg/mol, about 290 kg/mol, about 291 kg/mol, about 292 kg/mol, about 293 kg/mol, about 294 kg/mol, about 295 kg/mol, about 296 kg/mol, about 300 kg/mol, about 310 kg/mol, about 320 kg/mol, about 330 kg/mol, about 340 kg/mol, about 350 kg/mol, about 360 kg/mol, from about 291 to about 295 kg/mol, from about 291 kg/mol to about 296 kg/mol, from about 280 kg/mol to about 360 kg/mol, from about 290 kg/mol to about 340 kg/mol, from about 290 kg/mol to about 350 kg/mol, from about 300 kg/mol to about 345 kg/mol, or from about 290 kg/mol to about 330 kg/mol. Thus, in some embodiments, described herein is a method described for producing or purifying a glycosylated recombinant human lubricin characterized by a molecular weight of about 280 kg/mol, about 290 kg/mol, about 291 kg/mol, about 292 kg/mol, about 293 kg/mol, about 294 kg/mol, about 295 kg/mol, about 296 kg/mol, about 300 kg/mol, about 310 kg/mol, about 320 kg/mol, about 330 kg/mol, about 340 kg/mol, about 350 kg/mol, about 360 kg/mol, from about 291 to about 295 kg/mol, from about 291 kg/mol to about 296 kg/mol, from about 280 kg/mol to about 360 kg/mol, from about 290 kg/mol to about 340 kg/mol, from about 290 kg/mol to about 350 kg/mol, from about 300 kg/mol to about 345 kg/mol, or from about 290 kg/mol to about 330 kg/mol.

Lubricin function and activity can be assayed using any suitable method known in the art or a method described herein, for example, a cell adhesion assay, for example, an A375 cell adhesion assay. For example, lubricin function can be determined based on its ability to inhibit the adhesion of A375 human melanoma cells to the surface of cell-tissue culture microtiter plates. Thus, inhibition of adhesion of A375 cells in a dose-dependent manner by a recombinant lubricin sample compared to a reference substance can be used to determine recombinant lubricin function or activity. In some embodiments, a composition comprising recombinant lubricin purified using a method described herein shows activity of between 50% and 150% (for example, 50%, 75%, 100%, 125%, or 150%) relative to activity of a reference substance (for example, a reference sample of purified recombinant lubricin), as determined by a cell adhesion assay, for example, an A375 cell adhesion assay.

In some embodiments described herein, recombinant human lubricin activity (including, but not limited to, recombinant human lubricin obtained or purified using a method described herein), is assayed using a reporter cell assay, for example, an NF-κB reporter cell assay. For example, in some embodiments, recombinant human lubricin activity is assayed by analyzing modification of NF-κB activity in a reporter cell line, for example, THP1-Lucia™ NF-κB Cells (Cat. No. thp1-nfkb, InvivoGen, San Diego, Calif.) in response to recombinant human lubricin. In such embodiments, NF-κB activity can be monitored based on expression levels of a reporter gene, for example, a luciferase reporter gene or a modified luciferase reporter gene. In some embodiments, levels of reporter gene expression, for example, NF-κB-induced reporter gene expression, can be assessed using a suitable detection reagent, for example, QUANTI-Luc™ (Cat. No. rep-qlc1, InvivoGen, San Diego, Calif.) for detection of Lucia™. Thus, in some embodiments modification of NF-κB activity in response to a recombinant lubricin sample is compared to a reference substance to determine recombinant lubricin function or activity. In some embodiments, a composition comprising recombinant lubricin purified using a method described herein shows activity of between 50% and 150% (for example, 50%, 75%, 100%, 125%, or 150%) relative to activity of a reference substance (for example, a reference sample of purified recombinant lubricin), as determined by a reporter cell assay, for example, an NF-κB reporter cell assay.

The signal sequence of human lubricin is residues 1-24 of SEQ ID NO:1/SEQ ID NO:2. Accordingly, the mature form of human lubricin is residues 25-1404 of SEQ ID NO:1/SEQ ID NO:2.

TABLE 1
Sequence listing

| SEQ | | |
|---|---|---|
| ID | | |
| NO: | Description | Sequence |
| 1 | Lubricin, | MAWKTLPIYL LLLLSVFVIQ QVSSQDLSSC AGRCGEGYSR |
| | PRG4 protein; | DATCNCDYNC QHYMECCPDF KRVCTAELSC KGRCFESFER |
| | Isoform A | GRECDCDAQC KKYDKCCPDY ESFCAEVHNP TSPPSSKKAP |
| | NP_005798.3 | PPSGASQTIK STTKRSPKPP NKKKTKKVIE SEEITEEHSV |
| | | SENQESSSSS SSSSSSSTIR KIKSSKNSAA NRELQKKLKV |
| | | KDNKKNRTKK KPTPKPPVVD EAGSGLDNGD FKVTTPDTST |
| | | TQHNKVSTSP KITTAKPINP RPSLPPNSDT SKETSLTVNK |
| | | ETTVETKETT TTNKQTSTDG KEKTTSAKET QSIEKTSAKD |
| | | LAPTSKVLAK PTPKAETTTK GPALTTPKEP TPTTPKEPAS |
| | | TTPKEPTPTT IKSAPTTPKE PAPTTTKSAP TTPKEPAPTT |
| | | TKEPAPTTPK EPAPTTTKEP APTTTKSAPT TPKEPAPTTP |
| | | KKPAPTTPKE PAPTTPKEPT PTTPKEPAPT TKEPAPTTPK |
| | | EPAPTAPKKP APTTPKEPAP TTPKEPAPTT TKEPSPTTPK |
| | | EPAPTTTKSA PTTTKEPAPT TTKSAPTTPK EPSPTTTKEP |
| | | APTTPKEPAP TTPKKPAPTT PKEPAPTTPK EPAPTTTKKP |
| | | APTTPKEPAP TTPKETAPTT PKKLTPTTPE KLAPTTPEKP |
| | | APTTPEELAP TTPEEPTPTT PEEPAPTTPK AAAPNTPKEP |
| | | APTTPKEPAP TTPKEPAPTT PKETAPTTPK GTAPTTLKEP |
| | | APTTPKKPAP KELAPTTTKE PTSTTSDKPA PTTPKGTAPT |
| | | TPKEPAPTTP KEPAPTTPKG TAPTTLKEPA PTTPKKPAPK |
| | | ELAPTTTKGP TSTTSDKPAP TTPKETAPTT PKEPAPTTPK |
| | | KPAPTTPETP PPTTSEVSTP TTTKEPTTIH KSPDESTPEL |
| | | SAEPTPKALE NSPKEPGVPT TKTPAATKPE MTTTAKDKTT |
| | | ERDLRTTPET TTAAPKMTKE TATTTEKTTE SKITATTTQV |
| | | TSTTTQDTTP FKITTLKTTT LAPKVTTTKK TITTTEIMNK |
| | | PEETAKPKDR ATNSKATTPK PQKPTKAPKK PTSTKKPKTM |
| | | PRVRKPKTTP TPRKMTSTMP ELNPTSRIAE AMLQTTTRPN |
| | | QTPNSKLVEV NPKSEDAGGA EGETPHMLLR PHVFMPEVTP |
| | | DMDYLPRVPN QGIIINPMLS DETNICNGKP VDGLTTLRNG |
| | | TLVAFRGHYF WMLSPFSPPS PARRITEVWG IPSPIDTVFT |
| | | RCNCEGKTFF FKDSQYWRFT NDIKDAGYPK PIFKGFGGLT |
| | | GQIVAALSTA KYKNWPESVY FFKRGGSIQQ YIYKQEPVQK |
| | | CPGRRPALNY PVYGETTQVR RRRFERAIGP SQTHTIRIQY |
| | | SPARLAYQDK GVLHNEVKVS ILWRGLPNVV TSAISLPNIR |
| | | KPDGYDYYAF SKDQYYNIDV PSRTARAITT RSGQTLSKVW |
| | | YNCP |
| 2 | Lubricin, | MAWKTLPIYL LLLLSVFVIQ QVSSQDLSSC AGRCGEGYSR |
| | PRG4; | DATCNCDYNC QHYMECCPDF KRVCTAELSC KGRCFESFER |
| | Isoform | GRECDCDAQC KKYDKCCPDY ESFCAEVHNP TSPPSSKKAP |
| | CRA_a | PPSGASQTIK STTKRSPKPP NKKKTKKVIE SEEITEEHSV |
| | EAW91201.1 | SENQESSSSS SSSSSSSTIR KIKSSKNSAA NRELQKKLKV |
| | | KDNKKNRTKK KPTPKPPVVD EAGSGLDNGD FKVTTPDTST |
| | | TQHNKVSTSP KITTAKPINP RPSLPPNSDT SKETSLTVNK |
| | | ETTVETKETT TTNKQTSTDG KEKTTSAKET QSIEKTSAKD |
| | | LAPTSKVLAK PTPKAETTTK GPALTTPKEP TPTTPKEPAS |
| | | TTPKEPTPTT IKSAPTTPKE PAPTTTKSAP TTPKEPAPTT |
| | | TKEPAPTTPK EPAPTTTKEP APTTTKSAPT TPKEPAPTTP |
| | | KKPAPTTPKE PAPTTPKEPT PTTPKEPAPT TKEPAPTTPK |
| | | EPAPTAPKKP APTTPKEPAP TTPKEPAPTT TKEPSPTTPK |
| | | EPAPTTTKSA PTTTKEPAPT TTKSAPTTPK EPSPTTTKEP |
| | | APTTPKEPAP TTPKKPAPTT PKEPAPTTPK EPAPTTTKKP |
| | | APTTPKEPAP TTPKETAPTT PKKLTPTTPE KLAPTTPEKP |
| | | APTTPEELAP TTPEEPTPTT PEEPAPTTPK AAAPNTPKEP |
| | | APTTPKEPAP TTPKEPAPTT PKETAPTTPK GTAPTTLKEP |
| | | APTTPKKPAP KELAPTTTKE PTSTTCDKPA PTTPKGTAPT |
| | | TPKEPAPTTP KEPAPTTPKG TAPTTLKEPA PTTPKKPAPK |
| | | ELAPTTTKGP TSTTSDKPAP TTPKETAPTT PKEPAPTTPK |
| | | KPAPTTPETP PPTTSEVSTP TTTKEPTTIH KSPDESTPEL |
| | | SAEPTPKALE NSPKEPGVPT TKTPAATKPE MTTTAKDKTT |
| | | ERDLRTTPET TTAAPKMTKE TATTTEKTTE SKITATTTQV |
| | | TSTTTQDTTP FKITTLKTTT LAPKVTTTKK TITTTEIMNK |
| | | PEETAKPKDR ATNSKATTPK PQKPTKAPKK PTSTKKPKTM |
| | | PRVRKPKTTP TPRKMTSTMP ELNPTSRIAE AMLQTTTRPN |
| | | QTPNSKLVEV NPKSEDAGGA EGETPHMLLR PHVFMPEVTP |
| | | DMDYLPRVPN QGIIINPMLS DETNICNGKP VDGLTTLRNG |
| | | TLVAFRGHYF WMLSPFSPPS PARRITEVWG IPSPIDTVFT |
| | | RCNCEGKTFF FKDSQYWRFT NDIKDAGYPK PIFKGFGGLT |
| | | GQIVAALSTA KYKNWPESVY FFKRGGSIQQ YIYKQEPVQK |
| | | CPGRRPALNY PVYGETTQVR RRRFERAIGP SQTHTIRIQY |
| | | SPARLAYQDK GVLHNEVKVS ILWRGLPNVV TSAISLPNIR |
| | | KPDGYDYYAF SKDQYYNIDV PSRTARAITT RSGQTLSKVW |
| | | YNCP |
| 3 | Repeat | KEPAPTT |
| | sequence | |

In some embodiments, a method of the present invention is suitable for purifying a recombinantly produced glycosylated polypeptide having at least 85% sequence identity to the sequence of SEQ ID NO: 1 or 2, in particular at least 90%, e.g., at least 91%, at least 92%, at least 93%, at least 94%, at least 95%, at least 96%, at least 97%, at least 98%, at least 99% sequence identity to the sequence of SEQ ID NO: 1 or 2. In a specific embodiment, a method of the present invention is suitable for purifying a recombinantly produced glycosylated polypeptide having the sequence of SEQ ID NO: 1 or 2.

In some embodiments, methods of the present invention are suitable for purifying a recombinantly produced glycosylated polypeptide having at least 85% sequence identity to amino acids 25-1404 of SEQ ID NO: 1 or 2, in particular at least 90%, e.g., at least 91%, at least 92%, at least 93%, at least 94%, at least 95%, at least 96%, at least 97%, at least 98%, at least 99% sequence identity to amino acids 25-1404 of SEQ ID NO: 1 or 2. In a specific embodiment, a method of the present invention is suitable for purifying a recombinantly produced glycosylated polypeptide having the sequence amino acids 25-1404 of SEQ ID NO: 1 or 2.

The inventors have surprisingly found that a recombinantly produced glycosylated polypeptide, in particular a recombinantly produced glycosylated lubricin, of sufficient purity (e.g., sufficient to meet regulatory requirements for commercialization) and high yield (e.g., to meet commercially relevant demands) is obtainable by a method comprising three chromatography steps, in particular comprising: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC).

General chromatographic methods and their use are known to a person skilled in the art. See for example, Chromatography, 5th edition, Part A: Fundamentals and Techniques, Heftmann, E. (ed), Elsevier Science Publishing Company, New York, (1992); Advanced Chromatographic and Electromigration Methods in Biosciences, Deyl, Z. (ed.), Elsevier Science BV, Amsterdam, The Netherlands, (1998); Chromatography Today, Poole, C. F., and Poole, S. K., Elsevier Science Publishing Company, New York, (1991), Scopes, Protein Purification: Principles and Practice (1982); Sambrook, J., et al. (ed), Molecular Cloning: A Laboratory Manual, Second Edition, Cold Spring Harbor Laboratory Press, Cold Spring Harbor, N.Y., 1989; Current Protocols in Molecular Biology, Ausubel, F. M., et al. (eds)., John Wiley and Sons, Inc., New York; or Freitag, R., Chromatographical processes in the downstream processing of (recombinant) proteins, Meth. Biotechnol. 24 (2007) 421-453 (Animal cell biotechnology 2n Edition).

The term “ion exchange chromatography” as used within this application denotes a chromatography method which employs an “ion exchange chromatography material”. The term “ion exchange chromatography material” encompasses depending whether a cation is exchanged in a “cation exchange chromatography” a “cation exchange chromatography material” or an anion is exchanged in an “anion exchange chromatography” an “anion exchange chromatography material”.

The term “ion exchange chromatography material” as used within this application denotes an immobile high molecular weight solid phase that carries covalently bound charged groups as chromatographical functional groups. For overall charge neutrality not covalently bound counter ions are associated therewith. The “ion exchange chromatography material” has the ability to exchange its not covalently bound counter ions for similarly charged ions of the surrounding solution. Suitably, the cation exchange ligand may comprise a functional group selected from the list consisting of —OCH₂COO—, —CH₂CH₂CH₂SO₃—, and —CH₂SO₃—. Suitably, the cation exchange ligand may be selected from the list consisting of carboxymethyl (CM), sulphopropyl (SP), and methyl sulphonate (S). Suitably, the anion exchange ligand may comprise a functional group selected from the list consisting of —CH₂CHOHCHH₂N⁺H(CH₂CH₃)², —OCH₂CH₂N⁺H(CH₂CH₃)₂—, —OCH₂CH₂N⁺(C₂H₅)₂CH₂CH(OH)CH₃—, and —CH₂N⁺(CH₃)₃—. Suitably, the anion exchange ligand may be selected from the list consisting of diethylaminopropyl (ANX), diethylaminoethyl (DEAE), quaternary aminoethyl (QAE), quaternary ammonium (Q).

Depending on the chemical nature of the charged group the “ion exchange chromatography material” can additionally be classified as strong or weak ion exchange chromatography material, depending on the strength of the covalently bound charged substituent. For example, strong cation exchange chromatography materials have a sulfonic acid group as chromatographical functional group and weak cation exchange chromatography materials have a carboxylic acid group as chromatographical functional group.

### Multimodal Cation Exchange Chromatography (MCC)

The term “multimodal (or mixed mode) chromatography” or “MMC” as used herein refers to a chromatographic method in which solutes interact with stationary phase through more than one interaction mode or mechanism. MMC has been used as an alternative or complementary tool to traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography employs a combination of two or more of these interaction modes (Zhang K. and Lui X, 2016, Journal of Pharmaceutical and Biomedical Analysis, Volume 128, Pages 73-88).

One of the chromatographic steps utilized in the methods of the present invention is multimodal cation exchange chromatography (MCC).

“Multimodal cation exchange chromatography” or “MCC” as used herein refers to chromatographic methods that utilize a cation exchange and at least one more form of interaction between the stationary phase and analytes.

In some embodiments, the MCC resin utilized in methods of the present invention comprises a cation exchange ligand, e.g., a multimodal cation exchange ligand, which is able to interact with the recombinantly produced glycosylated polypeptide in an aqueous environment by ionic interaction. In some embodiments, the MCC resin utilized in a method of the present invention comprises a cation exchange ligand, in particular a weak cation exchange ligand, typically sulfonic acid (—SO4-) groups or carboxyl acid groups (—COO—). Suitably, the multimodal cation exchange ligand comprises carboxyl or other negatively charged group.

In some embodiments, the MCC resin utilized in a method of the present invention comprises a cation exchange ligand, in particular a weak cation exchange ligand, and a matrix. The matrix may be selected from the list consisting of agarose, cellulose, ceramics, dextran, polystyrene, polyacrilamide, silica, synthetic polymers, organic polymers. In some embodiments, the MCC resin utilized in a method of the present invention comprises a cation exchange ligand, in particular a weak cation exchange ligand, and an agarose matrix.

In some embodiments, the MCC resin utilized in a method of the present invention is selected from the following commercially available resins: Capto MMC™ (GE Healthcare; multimodal weak cation exchanger in combination with agarose matrix); Eshmuno®HCX (Merck Millipore; Eshmuno® cation exchanger hydrophilic polyvinyl ether base matrix); Toyopearl MX-Trp-650 M (TOSOH Bioscience; tryptophan ligand having weak carboxyl cation exchange and indole hydrophobic functional groups); Nuvia cPrime (BioRad); CHT Ceramic Hydroxyapatite (BioRad); and CFT Ceramic Fluoroapatite (Bio-Rad). In a preferred embodiment, the MCC resin utilized in a method of the present invention is Capto MMC resin.

Suitably, in some embodiments, the MCC step of methods of the invention is carried out in a bind-and-elute mode. The term “bind-and-elute mode” and grammatical equivalents thereof as used in the current invention denotes an operation mode of a chromatography method, in which a solution containing a substance of interest is brought in contact with a stationary phase, preferably a solid phase, whereby the substance of interest binds to the stationary phase. As a result the substance of interest is retained on the stationary phase whereas substances not of interest are removed with the flow-through or the supernatant. The substance of interest is afterwards eluted from the stationary phase in a second step and thereby recovered from the stationary phase with an elution solution. This does not necessarily denote that 100 percent of the substances not of interest are removed but essentially 100 percent or an acceptable portion of the substances not of interest are removed, e.g., at least 50 percent of the substances not of interest are removed, preferably at least 75 percent of the substances not of interest are removed, preferably at least 90 percent of the substances not of interest are removed, preferably more than 95 percent of the substances not of interest are removed.

Suitably, in some embodiments, the MCC step comprises the steps of (i) contacting the recombinantly produced glycosylated polypeptide composition with an MCC column comprising MCC resin, in particular loading the clarified cell culture supernatant onto an MCC column comprising MCC resin, (ii)washing the MCC resin, e.g., the MCC resin having the recombinantly produced glycosylated polypeptide bound thereto, with a washing buffer, and (iii) eluting the recombinantly produced glycosylated polypeptide containing fractions, in particular eluting the recombinantly produced glycosylated polypeptide containing tractions by an elution buffer comprising at least one amino acid which is positively charged at pH 8 to 10, in particular pH 9; and (iv) optionally, collecting the recombinantly produced glycosylated polypeptide containing fractions in purified or enriched form.

Suitably, in some embodiments, the MCC step comprises the steps of (i) contacting the recombinantly produced glycosylated polypeptide composition with an MCC column comprising MCC resin, in particular loading the clarified cell culture supernatant onto an MCC column comprising MCC resin, and (ii) eluting the recombinantly produced glycosylated polypeptide containing fractions by an elution buffer comprising at least one amino acid which is positively charged at pH 8 to 10, in particular pH 9, wherein the elution buffer is a high salt solution, in particular wherein the salt concentration is above 500 mM, e.g., 0.5 M to 2.5 M, 0.5 M to 2 M, 0.5 M to 1.5 M, 0.8 M to 1.2 M, in particular 0.9 M to 1.1 M.

In some embodiments, the elution buffer comprises sodium acetate and/or sodium chloride. In some embodiments, the elution buffer comprises between 10 mM and 100 mM sodium acetate, in particular 20 mM sodium acetate. In some embodiments, the elution buffer comprises between 0.5 M and 2 M sodium chloride, in particular 1 M sodium chloride. In a specific embodiment, the elution buffer comprises sodium acetate and sodium chloride. In a more specific embodiment, the elution buffer comprises between 10 mM and 100 mM sodium acetate, in particular 20 mM sodium acetate, and between 0.5 M and 2 M sodium chloride, in particular 1 M sodium chloride.

Suitably, the elution buffer comprises the amino acid which is positively charged at pH 8 to 10 and is selected from the group of amino groups containing amino acids such as lysine; arginine, histidine and combinations thereof, in particular in concentrations of at least 50 mM, e.g., 50 mM. In a specific embodiment, the elution buffer comprises L-arginine, in particular 50 mM L-arginine.

In some embodiments, the elution buffer further comprises about 15 mM to about 25 mM Tris (e.g., 15, 16, 17, 18, 19, 20, 21, 22, 23, 24, or 25 mM), in particular 20 mM Tris.

In some embodiments, the elution buffer has a pH between about 8.5 and about 10 (e.g., 8.5, 8.6, 8.7, 8.8, 8.9, 9.0, 9.1, 9.2, 9.3, 9.4, 9.5, 9.6, 9.7, 9.8, 9.9, or 10.0), in particular wherein the elution buffer has a pH 9.

In a specific embodiment, the elution buffer comprises 20 mM Tris, 20 mM sodium acetate, 50 mM L-arginine, 1 M NaCl at pH 9.

In some embodiments, a washing buffer is applied to the MCC resin, to wash away contaminants and retain the recombinantly produced glycosylated polypeptide, before the recombinantly produced glycosylated polypeptide is released. Suitably, the washing buffer is 20 mM Tris, pH 10.

In some embodiments, the MCC column is equilibrated with an equilibration buffer. In some embodiments, the MCC column is equilibrated with an equilibration buffer prior to contacting the recombinantly produced glycosylated polypeptide composition with the MCC column, e.g., prior to loading the clarified cell culture supernatant onto the MCC resin. In some embodiments, the equilibration buffer comprises 20 mM Tris, pH 8.

### Multimodal Anion Exchange Chromatography (MAC)

One of the chromatographic steps utilized in the methods of the present invention is multimodal anion exchange chromatography (MAC).

“Multimodal anion exchange chromatography” or “MAC” as used herein refers to chromatographic methods that utilize an anion exchange and at least one more form of interaction between the stationary phase and analytes. Suitable MAC resin may comprise any multi-modal anion exchange ligand, such as a ligand comprising amine or other positively charged groups.

In some embodiments, the MAC resin utilized in methods of the present invention comprises anion exchange ligand, e.g., multimodal anion exchange ligand, bound to a matrix, wherein the ligand is able to interact with the recombinantly produced glycosylated polypeptide and/or a contaminant in an aqueous environment by ionic interaction. In some embodiments, the multimodal anion exchange ligand interacts with a contaminant.

Suitably, the multimodal anion exchange ligand comprises amine or other positively charged groups. In some embodiments, the MAC resin is composed of a ligand comprising amine. Functional amines can be selected from the group consisting of primary, secondary, tertiary, and quaternary amines; hydrazine, such as mono-substituted hydrazine and di-substituted hydrazine; poly-amines; poly-imines; poly-Q (where Q refers to quaternary ammonium groups); aniline; octylamine and hydroxylamines. Stochastic resins are based on one type of amine group combined with different levels of phenyl groups, butyl groups, PEG, fluorine containing ligands and charged groups. Suitably, the MAC resin is composed of octylamine.

In some embodiments, the MAC resin utilized in methods of the present invention comprises an anion exchange ligand and a matrix. The matrix may be selected from the list consisting of agarose, cellulose, ceramics, dextran, polystyrene, polyacrilamide, silica, synthetic polymers, organic polymers. In some embodiments, the MAC resin utilized in methods of the present invention comprises an anion exchange ligand and an agarose matrix.

In some embodiments, the MAC resin utilized in methods of the present invention is selected from the following commercially available resins MEP Hypercel™ (Pall Corporation: 4-Mercapto-Ethyl-Pyridine (4-MEP) and cellulose matrix); PPA Hypercel™ (Pall Corporation; ligand: phenylpropylamine; electrostatic and hydrophobic interactions); HEA Hypercel™ (Pall Corporation; ligand: hexylamine; electrostatic and hydrophobic interactions); CaptoAdhere™ (GE Healthcare; ligand N-benzyl-n-methyl ethanolamine and agarose matrix); Capto Core 700™ (GE Healthcare; ligand octylamine and agarose matrix).

In some embodiments, the MAC resin is composed of a ligand-activated core, e.g., amine ligand, e.g., octylamine ligand, and inactive shell. The inactive shell has size exclusion properties, e.g., excludes large molecules such as the recombinantly produced glycosylated polypeptide, e.g., lubricin, from entering the core through the pores of the shell. Thus the recombinantly produced glycosylated polypeptide, e.g., lubricin, is collected in the column flow-through while smaller impurities bind to the internalized ligands. In a preferred embodiment, the MAC resin is Capto Core 700 resin, which provides tor 700 kDa molecular weight cut-off.

Suitably, in some embodiments, the MAC step of a method of the invention is carried out in a flow-through mode.

The term “flow-through mode” and grammatical equivalents thereof as used within the current invention denotes an operation mode of a chromatography method, in which a solution containing a substance of interest is brought in contact with a stationary phase, preferably a solid phase, whereby the substance of interest does not bind to that stationary phase. In flow-through mode, the pH of the sample and buffer can be selected to modify the charge of the target protein or the chromatography resin such that the target protein is directly maintained in the flow-through fractions while the impurities are bound to the resin. As a result the substance of interest is obtained either in the flow-through or the supernatant. Substances not of interest, which were also present in the solution, bind to the stationary phase and are removed from the solution. This does not necessarily denote that 100 percent of the substances not of interest are removed from the solution but essentially 100 percent of the substances not of interest are removed, e.g., at least 50 percent of the substances not of interest are removed from the solution, preferably at least 75 percent of the substances not of interest are removed from the solution, preferably at least 90 percent of the substances not of interest are removed from the solution, preferably more than 95 percent of the substances not of interest are removed from the solution.

Suitably, in some embodiments, the MAC step of a method of the present invention comprises the steps of (i) contacting the recombinantly produced glycosylated polypeptide composition with a MAC column comprising MAC resin, in particular loading the eluate of the MCC step onto a MAC column comprising MAC resin, (ii) washing the MAC resin with a washing buffer, and (iii) collecting the flow-through comprising the recombinantly produced glycosylated polypeptide in purified or enriched form.

Suitably, in some embodiments, the MAC step of a method of the present invention comprises the steps of contacting the recombinantly produced glycosylated polypeptide composition with a MAC column comprising MAC resin, in particular loading the eluate of the MCC step onto a MAC column comprising MAC resin. In some embodiments, the recombinantly produced glycosylated polypeptide composition, e.g., the eluate of the MCC step, is pH-adjusted to 6-9 (e.g., 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0), in particular pH 7.0, prior to being loaded onto the MAC column.

In some embodiments, the MAC column is equilibrated with an equilibration buffer. In some embodiments, the MAC column is equilibrated with an equilibration buffer prior to contacting the recombinantly produced glycosylated polypeptide composition with the MAC column. Suitably, the equilibration buffer comprises about 40 to about 60 mM Na phosphate, about 300 to about 1000 mM NaCl at pH 6 to 9 (e.g., 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0) in particular wherein the equilibration buffer comprises 50 mM Na phosphate, 750 mM NaCl at pH 7.

Suitably, in some embodiments, the MAC step of a method of the present invention comprises the steps of (i) contacting the recombinantly produced glycosylated polypeptide composition with a MAC column comprising MAC resin, in particular loading the eluate of the MCC step onto a MAC column comprising MAC resin,, (ii) washing the MAC resin with a washing buffer, and (iii) collecting the flow-through comprising the recombinantly produced glycosylated polypeptide in purified or enriched form. Suitably, the washing buffer comprises about 40 to about 60 mM Na phosphate, about 300 to about 1000 mM NaCl at pH 6-9 (e.g., 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0), in particular wherein the washing buffer comprises 50 mM Na phosphate, 750 mM NaCl at pH 7.

### Hydrophobic Interaction Chromatography (HIC).

One of the chromatographic steps utilized in the methods of the present invention is hydrophobic interaction chromatography (HIC).

The terms “hydrophobic interaction chromatography” or “HIC” refer to a chromatography method in which a “hydrophobic interaction chromatography material” is employed. HIC is based on the adsorption of biomolecules to a weakly hydrophobic surface at high salt concentrations, followed by elution with a descending salt gradient. This technique exploits hydrophobic regions present on the surface of biomolecules that bind to immobilized hydrophobic ligands on chromatography supports.

A “hydrophobic interaction chromatography material” is a chromatography material to which hydrophobic groups, such as butyl-, octyl-, or phenyl-groups, are bound as chromatographical functional groups. The polypeptides are separated depending on the hydrophobicity of their surface exposed amino acid side chains, which can interact with the hydrophobic groups of the hydrophobic interaction chromatography material. The interactions between polypeptides and the chromatography material can be influenced by temperature, solvent, and ionic strength of the solvent. A temperature increase, e.g., supports the interaction between the polypeptide and the hydrophobic interaction chromatography material as the motion of the amino acid side chains increases and hydrophobic amino acid side chains buried inside the polypeptide at lower temperatures become accessible. The hydrophobic interaction is also promoted by kosmotropic salts and decreased by chaotropic salts. “Hydrophobic interaction chromatography materials” include, e.g., Phenylsepharose CL-4B, 6 FF, HP, Phenyl Superose, Octylsepharose CL-4B,4 FF, and Butylsepharose 4 FF, Hexyl, Ether, PPG (all available from Amersham Pharmacia Biotech Europe GmbH, Germany), which are obtained via glycidyl-ether coupling to the bulk material.

In some embodiments, the HIC column comprises phenyl membrane adsorber, in particular Sartobind Phenyl membrane adsorber. The phenyl membrane adsorber follows the same rules known from the conventional hydrophobic interaction chromatography. Due to the large pore size, membrane adsorbers show excellent flow properties. There is almost no diffusion limitation of mass transport compared with conventional bead chromatography. Buffers with high concentrations of salt promote the adsorption of proteins on the hydrophobic membrane matrix. Proteins are eluted by decreasing the salt concentration in the elution buffer.

In some embodiments, the HIC step is carried out in a flow-through mode.

In some embodiments, the HIC step comprises the steps of contacting the recombinantly produced glycosylated polypeptide composition with a HIC column, e.g., loading the flow-through of the MAC step onto a HIC column.

Suitably, the recombinantly produced glycosylated polypeptide composition, e.g. the flow-through of the MAC step, is adjusted to high salt concentration prior to the loading the recombinantly produced glycosylated polypeptide composition, e.g. the flow-through onto the HIC column, in particular wherein the resulting salt concentration is above 500 mM, e.g., 0.5 M to about 1 M, 0.5 M to 1.5 M, 0.5 M to 1.2 M, 0.8 M to 1 M, in particular about 0.9 M. In some embodiments, the salt is ammonium sulfate.

In some embodiments, a method described herein further comprises performing depth filtration after adjusting the recombinantly produced glycosylated polypeptide composition to high salt concentration and prior to loading the recombinantly produced glycosylated polypeptide composition onto the HIC column. Thus, in some embodiments, a method described herein further comprises steps of adjusting the recombinantly produced glycosylated polypeptide composition to high salt concentration after the MAC step and then performing depth filtration prior to loading the recombinantly produced glycosylated polypeptide composition onto the HIC column. In some embodiments, depth filtration is performed using a suitable filter, for example, a cellulose or polypropylene fiber-based filter, for example, a positively charged triple layer BIHC filter.

In some embodiments, the HIC column is equilibrated with an equilibration buffer. In some embodiments, the HIC column is equilibrated with an equilibration buffer prior to contacting the recombinantly produced glycosylated polypeptide composition with the HIC column. In certain embodiments, the HIC column is equilibrated with the equilibration buffer comprising 15-25 mM Na phosphate, 500-1500 mM ammonium sulfate at pH 6.5-7.5 (e.g., 6.5, 6.6, 6.7, 6.8, 6.9, 7.0, 7.1, 7.2, 7.3, 7.4, 7.5), in particular wherein the equilibration buffer comprises 20 mM Na phosphate, 1 M ammonium sulfate at pH 7.

Suitably, in some embodiments, the HIC step of the method of the present invention comprises the step of washing the HIC column with a washing buffer. Suitably, in some embodiments, the HIC step of the method of the present invention comprises washing the HIC column with a washing buffer after the contacting the recombinantly produced glycosylated polypeptide composition with the HIC column, e.g., loading of the flow-through pool the second chromatographic step thereto. In some embodiments, the washing buffer comprises 15-25 mM Na phosphate, 500-1500 mM ammonium sulfate at pH 6.5-7.5 (e.g., 6.5, 6.6, 6.7, 6.8, 6.9, 7.0, 7.1, 7.2, 7.3, 7.4, 7.5), in particular wherein the washing buffer comprises 20 mM Na phosphate, 1 M ammonium sulfate at pH 7.

### Endonuclease

In some embodiments, a method of the present invention comprises an additional step(s) (e.g., prior to the first chromatography step) to degrade nucleic acid molecules (e.g., DNA and/or RNA) present in the cell culture or eluate. Thus, in some embodiments, the method of the invention comprises treating the cell culture or eluate with an endonuclease (for example, Benzonase® nuclease) and MgCl₂. Suitably, Benzonase® nuclease is added to a cell culture producing the glycosylated polypeptide. Alternatively, Benzonase® nuclease is added to a clarified cell culture supernatant comprising the recombinantly produced glycosylated polypeptide. In some embodiments, the Benzonase® nuclease is subjected to filtration with a 0.2 μm filter. In some embodiments, the step of Benzonase® nuclease treatment comprises adding Mg²⁺ to the cell culture or to the clarified cell culture supernatant, in particular to the amount of 1-2 mM Mg²⁺ (for example, adding 1-2 mM MgCl₂, for example, adding 1-2 mM MgCl₂ subjected to filtration with a 0.2 μm filter). In some embodiments, the step of treating the cell culture or the clarified cell culture supernatant with Benzonase® nuclease comprises adding from 5 U to 50 U, of Benzonase® nuclease per 1 ml of the cell culture supernatant. In a more specific embodiment, the step of treating the cell culture or the clarified cell culture supernatant with Benzonase® nuclease comprises (i) adding from about 5 U to 50 U of Benzonase® nuclease per 1 ml of the cell culture supernatant (e.g., about 200 μl of Benzonase® (250 U/μl) per 1.0 kg of clarified harvest), and (ii) adding Mg²⁺ to the cell culture or to the clarified cell culture supernatant, in particular to the amount of about 1 mM Mg²±. In an embodiment described herein, the step of treating the clarified cell culture supernatant with Benzonase® nuclease comprises (i) adding between 1 U to 5 U (for example, 2 U) of Benzonase® nuclease per 1 ml of the cell culture supernatant, and (ii) adding Mg²⁺ to the clarified cell culture supernatant, in particular to the amount of about 1 mM Mg²⁺. Also in an embodiment described herein, the step of treating the cell culture with Benzonase® nuclease comprises (i) adding between 1 U to 5 U (for example, 2 U) of Benzonase® nuclease per 1 ml of the cell culture, and (ii) adding Mg²⁺ to the cell culture, in particular to the amount of about 1 mM Mg²⁺.

In some embodiments, the step of treating the clarified cell culture supernatant with Benzonase® nuclease comprises (i) adding 2 U, 5 U, 10 U, 15 U, 20 U, 30 U, 40 U, 50 U, between 5 U and 50 U, between 1 U and 5 U, or between 1 U and 50 U of Benzonase® nuclease per 1 ml of the cell culture supernatant, and (ii) adding MgCl₂to the clarified cell culture supernatant, in particular to the amount of about 1 mM MgCl₂. In some embodiments, the step of treating the clarified cell culture supernatant with Benzonase® nuclease comprises (i) adding 2 U of Benzonase® nuclease per 1 ml of the cell culture supernatant, and (ii) adding MgCl₂ to the cell culture or to the clarified cell culture supernatant, in particular to the amount of about 1 mM MgCl₂. In some embodiments, the cell culture harvest is cooled to 2-8° C. before being contacted with MgCl₂ and the endonuclease.

In some embodiments, the step of treating the cell culture with Benzonase® nuclease comprises adding 2 U, 5 U, 10 U, 15 U, 20 U, 30 U, 40 U, 50 U, between 5 U and 50 U, between 1 U and 5 U, or between 1 U and 50 U of Benzonase® nuclease per 1 ml of the cell culture and MgCl₂ to the cell culture. In some embodiments, the method of the invention comprises adding 2 U of Benzonase® nuclease per 1 ml of the cell culture and MgCl₂ to the cell culture. In some embodiments, the cell culture is cooled to 2-8° C. before being contacted with MgCl₂ and the endonuclease.

In some embodiments, the step of treating the cell culture with an endonuclease comprises treating the cell culture with an endonuclease (for example, Benzonase® nuclease) and MgCl₂, for example, treating the cell culture with an endonuclease (for example, Benzonase® nuclease) and MgCl₂ on day 9, day 10, day 11, day 12, day 13, day 14, day 15 of the cell culture (for example, on day 9, day 10, day 11, day 12, day 13, day 14, day 15 of culturing cells in a bioreactor, for example, culturing cells in a bioreactor at 30° C.). In some embodiments, the method of the invention comprises adding 2 U, 5 U, 10 U, 15 U, 20 U, 30 U, 40 U, 50 U, between 5 U and 50 U, between 1 U and 5 U, or between 1 U and 50 U of Benzonase® nuclease per 1 ml of the cell culture and MgCl₂ to the cell culture. In some embodiments, the method of the invention comprises adding 2 U of Benzonase® nuclease per 1 ml of the cell culture and MgCl₂ to the cell culture.

In some embodiments, the method of the present invention comprises: (a) cultivating a host cell, in particular a eukaryotic cell, comprising a nucleic acid encoding a recombinantly produced lubricin glycoprotein; (b) clarifying cell culture supernatant comprising the recombinantly produced lubricin glycoprotein; and (c) purifying said recombinantly produced lubricin glycoprotein with a method according to the present invention.

### Viral Inactivation

In some embodiments, the method of the present invention further comprises a virus inactivation and/or virus filtration step being carried out between one or more of the chromatographic steps or after the third chromatographic step.

In certain embodiments, the methods of the invention further comprise one or more viral inactivation (VIN) treatment steps and viral removal steps as described herein. Various methods of virus inactivation are known to those of skill in the art and can be used in a method of the invention, including but not limited to pasteurization, terminal dry heat, vapor heat, solvent/detergents, and acid pH. Virus removal procedures are also well known, including but not limited to precipitation, chromatography, and nanofiltration. Viral inactivation and removal can be done in-process (e.g., nanofiltration and solvent/detergent treatment, pasteurization, steam-treatment, and/or incubation at about pH 4.0) or terminal in the final container (e.g., terminal pasteurization or terminal dry-heat treatment).

In some embodiments, a method of the present invention comprises a step of virus inactivation by low pH, e.g., pH 4.0 or less, such as a pH of about 3.4, 3.5, 3.6, 3.7, 3.8, 3.9, or 4.0. In a specific embodiment, the step of virus inactivation is carried out between the second and the third chromatographic steps (for example, after a multimodal anion exchange chromatography (MAC) step and before a hydrophobic interaction chromatography (HIC) step).

In some embodiments, a method of the present invention includes a step of virus removal following a step of virus inactivation, for example, virus inactivation by low pH. In some embodiments, the step of virus removal is performed after a step of virus inactivation and before a hydrophobic interaction chromatography (HIC) step. In some embodiments, the virus removal step comprises subjecting a solution (for example, a filtrate) comprising a highly glycosylated protein (for example, a recombinant human lubricin) obtained from a virus inactivation step to tangial flow filtration using a sodium chloride containing buffer. In some embodiments, the solution is characterized by a conductivity of greater than 50 mS/cm. In some embodiments, the sodium chloride containing buffer has a pH of between pH 6.5 and pH 7.5 (for example, pH 6.5, pH 6.6, pH 6.7, pH 6.8, pH 6.9, pH 7.0, pH 7.1, pH 7.2, pH 7.3, pH 7.4, or pH 7.5). In some embodiments, the tangial flow filtration is performed until a conductivity 15 mS/cm or less (for example, 15 mS/cm, 10 mS/cm, or 5 mS/cm) is achieved. In some embodiments, following tangial flow filtration, the solution is filtered over an anion exchange depth filter (AEX). In some embodiments, the AEX filter is washed with the buffer used for the tangial flow filtration. In some embodiments, filtrate from the AEX filter is treated with ammonium sulfate solution to achieve an ammonium sulfate concentration of from 0.4 M to 1.5M (for example, 0.4 M, 0.5 M, 0.6 M, 0.7 M, 0.8 M, 0.9 M, 1.0 M, 1.1 M, 1.2 M, 1.3 M, 1.4 M, or 1.5 M). In some embodiments, the filtrate from the AEX filter treated with ammonium sulfate solution is subjected to an HIC step. Thus, in some embodiments of the invention described herein, a method of purifying a highly glycosylated protein (for example, a recombinant human lubricin) comprises a step of virus removal as described above. In some embodiments of the invention described herein, a method of producing a highly glycosylated protein (for example, a recombinant human lubricin) comprises a step of virus removal as described above. Also described herein is a highly glycosylated protein (for example, a recombinant human lubricin) produced or purified by a method that comprises a step of virus removal as described above. Also described herein is a composition comprising a highly glycosylated protein (for example, a recombinant human lubricin) produced or purified by a method that comprises a step of virus removal as described above.

In some embodiments, a method of the present invention comprises a step of virus inactivation wherein an eluate is contacted with a detergent or N,N-Dimethylurea (DMU). In one aspect, an eluate is contacted with N,N-Dimethylurea (DMU), tor example, a solution of about 3 M N,N-Dimethylurea (DMU).

In some embodiments, a method of the present invention comprises a step of virus filtration. In a specific embodiment, the virus filtration is carried out after the third chromatographic step (for example, after an HIC step). Virus filtration methods and filters are well known to those of skill in the art, and include but are not limited to use of microfiltration (e.g., membranes with pore size of about 0.1 to 10 μm) and ultrafiltration (e.g., membranes with pore size of about 0.001 to 0.1 μm) to capture virus particles.

In some embodiments, a method of the present invention further comprises a step of performing buffer exchange by ultrafiltration/diafiltration (UF/DF). In a specific embodiment, the step of buffer exchange by UF/DF is carried out after the third chromatographic step (for example, after an HIC step). In a more specific embodiment, the step of buffer exchange by UF/DF is carried out after the third chromatographic step after the step of virus filtration.

### Purity Parameters

In some embodiments, the content of contaminants (e.g., polynucleotide and/or host cell protein) is reduced in the polypeptide solution obtained after performance of the three chromatography steps compared to the content prior to the purification, e.g., prior to the first chromatography step. In some embodiments, the content of contaminants (e.g., polynucleotide and/or host cell protein) is reduced in the polypeptide solution obtained after the third chromatography step compared to the content prior to the first chromatography step. In a further embodiment, the content of contaminants (e.g., polynucleotide and/or host cell protein) is reduced in the polypeptide solution obtained after the third chromatography step compared to the content prior to the Benzonase® nuclease treatment step. In some embodiments, the content of contaminants (e.g., polynucleotide and/or host cell protein) is reduced in the polypeptide solution obtained after preforming a depth filtration step, for example, a depth filtration step performed prior to HIC, for example, a depth filtration step performed after an MAC step and before an HIC step. In general, depth filtration utilizes thickness of the filtration media (e.g., cellulose) to trap suspended particles (for example, suspended host cell protein particles) and separate them from a carrying fluid.

In some embodiments, the purity of the final polypeptide solution obtained after the last purification step of the method of the present invention is >4000 IU/mg, preferably >9000 IU/mg and more preferably >10 000 IU/mg protein and that the DNA content is <1000 pg/1000 IU the recombinantly produced glycosylated polypeptide, preferably <100 pg/1000 IU the recombinantly produced glycosylated polypeptide and more preferably <10 pg/1000 IU the recombinantly produced glycosylated polypeptide.

In some embodiments, at least 30%, e.g., at least 35%, at least 40%, at least 45%, at least 50%, at least 55%, at least 60% at least 65%, at least 70%, at least 75%, at least 80%, of the recombinantly produced polypeptide is recovered after the method of the invention. In some embodiments, at least 45%, e.g., at least 50%, at least 55%, at least 60%, at least 65%, at least 70%, at least 75%, at least 80%, of the recombinantly produced polypeptide is recovered after the first chromatography step compared to the amount prior to the first chromatography step. In some embodiments, at least 80%, e.g., at least 85%, at least 90%, at least 95%, of the recombinantly produced polypeptide is recovered after the second chromatography step compared to the amount prior to the second chromatography step. In some embodiments, at least 90%, e.g., at least 95%, of the recombinantly produced polypeptide is recovered after the third chromatography step compared to the amount prior to the third chromatography step.

### Purity Criteria Measured by SEC, RPC, and rCE-SDS

Purity of a solution (including drug substance or drug product) is a quality criteria that can be measured by: a size-exclusion chromatograph (SEC) assay; a reversed-phase chromatography (RPC) assay; a reducing capillary electrophoresis under denaturing conditions (rCE-SDS) assay; or any combination of SEC, RPC, and rCD-SDS assays as described herein. The purity of a solution (e.g., an eluate) is the percentage of the target protein in relation to the overall peak including aggregates and degradation products.

For example, in some embodiments, purity of a solution comprising a recombinantly produced glycosylated polypeptide purified by a method of the invention can be determined using reversed phase chromatography (RPC). RPC is a chromatography technique that relies on a hydrophobic stationary phase and a polar mobile phase for protein purification. A highly glycosylated polypeptide purified by a method of the invention can be resolved as two major groups of peaks by RPC in ion-pair mode with UV-detection. The peak area of the two major groups of peaks versus the total peak area defines purity expressed as relative peak area percentage. In some embodiments, purity of a solution comprising a recombinantly produced glycosylated polypeptide produced by a method described herein is 80% or greater, 85% or greater, 90% or greater, 95% or greater, 96% or greater, 97% or greater, 98% or greater, 99% or greater, 99.5% or greater, 99.6% or greater, 99.7% or greater, 99.8% or greater, or 99.9% or greater, as determined by RPC. Thus, also described herein is a method of determining purity of a composition (for example, a pharmaceutical composition) comprising a recombinantly produced glycosylated polypeptide (for example, recombinant human lubricin) purified using a method described herein, wherein the method comprises steps of performing reversed phase chromatography (RPC) and calculating purity of the composition, as determined by RPC. In some embodiments, purity of the composition is calculated as a percent purity, as determined by RPC (for example, 80% or greater, 85% or greater, 90% or greater, 95% or greater, 96% or greater, 97% or greater, 98% or greater, 99% or greater, 99.5% or greater, 99.6% or greater, 99.7% or greater, 99.8% or greater, 99.9% or greater, about 80%, about 85%, about 90%, about 95%, about 96%, about 97%, about 98%, about 99%, about 99.5%, about 99.6%, about 99.7%, about 99.8%, or about 99.9%).

In some embodiments, purity of a solution comprising a recombinantly produced glycosylated polypeptide purified by a method of the invention can be determined using size exclusion chromatography (SEC). SEC is a chromatography technique that separates molecules based on size and which can be used to measure, for example, aggregates and fragments of a purified protein product. Aggregates of a purified protein product can be separated from monomer based on size under native conditions by SEC and detected with UV detection. The amount of aggregate is determined as a percentage of the total area obtained for each sample. In some embodiments, the sum of aggregates of the recombinantly produced glycosylated polypeptide produced by a method described herein (for example, the sum of aggregates in a composition comprising a recombinantly produced glycosylated polypeptide produced by a method described herein) is about 10% or less, about 5% or less, about 4% or less, about 3% or less, about 2% or less, or about 1% or less, preferably less than 1%, as determined by SEC (for example, about 10% or less, about 5% or less, about 4% or less, about 3% or less, about 2% or less, or about 1% or less, preferably less than 1% of the total amount of recombinantly produced glycosylated polypeptide, as determined by SEC). Thus, also described herein is a method of determining the percent of aggregates of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing size exclusion chromatography (SEC) and calculating the percent of aggregates, as determined by SEC.

In some embodiments, the sum of fragments of the recombinantly produced glycosylated polypeptide produced by a method described herein (for example, the sum of fragments in a composition comprising a recombinantly produced glycosylated polypeptide produced by a method described herein) is about 15% or less, about 10% or less, about 5% or less, about 4% or less, about 3% or less, about 2% or less, about 1.5% or less, or about 1% or less, preferably less than 1%, as determined by SEC (for example, about 15% or less, about 10% or less, about 5% or less, about 4% or less, about 3% or less, about 2% or less, about 1.5% or less, or about 1% or less, preferably less than 1% of the total amount of recombinantly produced glycosylated polypeptide, as determined by SEC). Thus, also described herein is a method of determining the percent of fragments of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing size exclusion chromatography (SEC) and calculating the percent of fragments, as determined by SEC.

In some embodiments, the sum of purified monomers of the recombinantly produced glycosylated polypeptide produced by a method described herein is 70% or more, 75% or more, 80% or more, 85% or more, 95% or more, 96% or more, 97% or more, 98% or more, 99% or more, 99.5% or more, 99.6% or more, 99.7% or more, 99.8% or more, or 99.9% or more, as determined by SEC (for example, 70% or more, 75% or more, 80% or more, 85% or more, 95% or more, 96% or more, 97% or more, 98% or more, 99% or more, 99.5% or more, 99.6% or more, 99.7% or more, 99.8% or more, or 99.9% or more of the total amount of recombinantly produced glycosylated polypeptide, as determined by SEC). Thus, also described herein is a method of determining the percent of purified monomers of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing size exclusion chromatography (SEC) and calculating the percent of monomers of the recombinantly produced glycosylated polypeptide, as determined by SEC.

Size exclusion chromatography (SEC) with UV detection can also be used to calculate protein quantity based on total sample peak area versus total peak area of a reference of known concentration. In some embodiments, protein concentration of a composition comprising a glycosylated polypeptide purified by a method of the invention is between 1.50 mg/ml and 3.00 mg/ml. In some embodiments, protein concentration of a composition comprising a glycosylated polypeptide purified by a method of the invention is between 1.60 mg/ml and 2.40 mg/ml. For example, in some embodiments, protein concentration of a composition comprising a glycosylated polypeptide purified by a method of the invention is 1.50 mg/ml, 1.60 mg/ml, 1.70 mg/ml, 1.80 mg/ml, 1.90 mg/ml, 2.00 mg/ml, 2.10 mg/ml, 2.20 mg/ml, 2.30 mg/ml, 2.40 mg/ml, 2.50 mg/ml, or 3.00 mg/ml. Thus, also described herein is a method of determining the concentration of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing size exclusion chromatography (SEC) and determining protein concentration of glycosylated polypeptide in the composition. In some embodiments, the protein concentration of glycosylated polypeptide in the composition is 1.50 mg/ml, 1.60 mg/ml, 1.70 mg/ml, 1.80 mg/ml, 1.90 mg/ml, 2.00 mg/ml, 2.10 mg/ml, 2.20 mg/ml, 2.30 mg/ml, 2.40 mg/ml, 2.50 mg/ml, 3.00 mg/ml, or between 1.60 mg/ml and 2.40 mg/ml, as determined by SEC.

In some embodiments of the invention, the methods described herein include a step comprising performing reducing capillary electrophoresis under denaturing conditions (rCE-SDS). In such embodiments, rhlubricin polypeptides and fragments thereof are denatured with sodium dodecyl sulfate (SDS) and reduced with mercaptoethanol. Without being bound by theory, it is believes that SDS masks the intrinsic charge of the proteins and forms complexes with a constant charge per unit mass. These complexes are separated according to their size by migration through a hydrophilic sieving polymer in an electric field. The main peak and the variants are quantified by relative time-corrected peak area determination. rCE-SDS can be used to remove protein fragments and increase purity of a composition.

In some embodiments, purity of a solution comprising a recombinantly produced glycosylated polypeptide purified by a method of the invention can be determined by reducing capillary electrophoresis under denaturing conditions (rCE-SDS). rCE-SDS is a chromatography technique in which polypeptides are denatured with sodium dodecyl sulfate (SDS) and reduced with mercaptoethanol. Without being bound by theory, it is believes that SDS masks the intrinsic charge of the polypeptides and forms complexes with a constant charge per unit mass. These complexes are separated according to their size by migration through a hydrophilic sieving polymer in an electric field. The main peak and the variants are quantified by relative time-corrected peak area determination. rCE-SDS can be used to measure purity (for example, the percentage of low molecular weight impurities, e.g., protein fragments) in a composition, for example, a composition comprising a recombinantly produced glycosylated polypeptide, for example recombinant human lubricin. In some embodiments, the purity of the recombinantly produced glycosylated polypeptide produced or purified by a method described herein (for example, the sum of protein fragments in a composition comprising a recombinantly produced glycosylated polypeptide produced by a method described herein) is about 10% or less, about 5% or less, about 4% or less, about 3% or less, about 2% or less, or about 1% or less, preferably less than 1%, as determined by rCE-SDS (for example, about 10% or less, about 5% or less, about 4% or less, about 3% or less, about 2% or less, or about 1% or less, preferably less than 1% of the total amount of recombinantly produced glycosylated polypeptide, as determined by rCE-SDS). Thus, also described herein is a method of determining the percent of protein fragments of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing rCE-SDS and calculating the percent of protein fragments, as determined by rCE-SDS. Also described herein is a method of determining the percent purity of a recombinantly produced glycosylated polypeptide (for example, recombinant human lubricin) in a composition (for example, a pharmaceutical composition) comprising the recombinantly produced glycosylated polypeptide purified using a method described herein, wherein the method comprises steps of performing rCE-SDS and calculating the percent purity, as determined by rCE-SDS.

### Purified Proteins

In a further aspect, the present invention relates to a recombinantly produced glycosylated polypeptide purified by a method of the invention. In a specific embodiment, the present invention relates to lubricin purified by a method of the invention. In some embodiments, the present invention relates to a recombinantly produced glycosylated polypeptide purified by a method of the invention, wherein the recombinantly produced glycosylated polypeptide has at least 80% sequence identity to SEQ ID NO: 1 or 2, e.g., at least 85%, in particular at least 90%, e.g., at least 91%, at least 92%, at least 93%, at least 94%, at least 95%, at least 96%, at least 97%, at least 98%, or at least 99% sequence identity to amino acids 25-1404 of SEQ ID NO: 1 or 2. In a specific embodiment, the present invention relates to a recombinantly produced glycosylated polypeptide purified by a method of the invention, wherein the recombinantly produced glycosylated polypeptide has amino acids 25-1404 of SEQ ID NO: 1 or 2.

In another aspect, the present invention relates to a pharmaceutical composition comprising a recombinantly produced glycosylated polypeptide, e.g., lubricin, purified by a method of the invention. In some embodiments, the present invention relates to a pharmaceutical composition comprising a substantially pure recombinantly produced glycosylated polypeptide, e.g., lubricin, purified by a method of the invention. In some embodiments, the recombinantly produced glycosylated polypeptide purified by a method of the invention has at least 85% sequence identity to SEQ ID NO: 1 or 2, in particular at least 90%, e.g., at least 91%, at least 92%, at least 93%, at least 94%, at least 95%, at least 96%, at least 97%, at least 98%, or at least 99% sequence identity to amino acids 25-1404 of SEQ Ill NO: 1 or 2. In a specific embodiment, the recombinantly produced glycosylated polypeptide purified by a method of the invention comprises amino acids 25-1404 of SEQ ID NO: 1 or 2.

As used herein, the term “substantially pure” with reference to a recombinantly produced glycosylated polypeptide means that the recombinantly produced glycosylated polypeptide includes less than 10%, preferably less than 5%, more preferably less than 3%, more preferably less than 1%, most preferably less than 0.1% by weight of any remaining contaminants (e.g., polynucleotides, host proteins, target protein aggregates, and/or process impurities arising from its preparation). For example, the recombinantly produced glycosylated polypeptide may be deemed substantially pure in that it has a purity greater than 90 weight %, as measured by means that are at this time known and generally accepted in the art, where the remaining less than 10 weight % of material comprises contaminants (e.g., polynucleotides, host proteins, target protein aggregates, and/or process related impurities). The presence of reaction impurities and/or processing impurities may be determined by analytical techniques known in the art, such as, for example, chromatography, mass spectrometry, or qPCR.

Protein concentration of a sample at any stage of purification can be determined by any suitable method. Such methods are well known in the art and include: 1) colorimetric methods such as the Lowry assay, the Bradford assay, the Smith assay, and the colloidal gold assay; 2) methods utilizing the UV absorption properties of proteins (for example, chromatographic methods utilizing UV absorption); and 3) visual estimation based on stained protein bands on gels relying on comparison with protein standards of known quantity on the same gel. See e.g. Stoschek (1990), Quantitation of Protein, in Guide to Protein Purification, Methods in Enzymol. 182: 50-68.

The target protein, as well as contaminating proteins that may be present in a sample, can be monitored by any appropriate means. Preferably, the technique should be sensitive enough to detect contaminants in the range between about 2 parts per million (ppm) (calculated as nanograms per milligram of the protein being purified) and 500 ppm. For example, enzyme-linked immunosorbent assay (ELISA), a method well known in the art, may be used to detect contamination of the protein by the second protein. See e.g. Reen (1994), Enzyme-Linked Immunosorbent Assay (ELISA), in Basic Protein and Peptide Protocols, Methods Mol. Biol. 32: 461-466, which is incorporated herein by reference in its entirety. In one aspect, contamination of the protein by such other proteins can be reduced after the methods described herein, preferably by at least about two-fold, more preferably by at least about three-fold, more preferably by at least about five-fold, more preferably by at least about ten-fold, more preferably by at least about twenty-fold, more preferably by at least about thirty-fold, more preferably by at least about forty-fold, more preferably by at least about fifty-fold, more preferably by at least about sixty-fold, more preferably by at least about seventy-fold, more preferably by at least about 80-fold, more preferably by at least about 90-fold, and most preferably by at least about 100-fold.

In another aspect, contamination of the target protein by other, contaminating proteins after the methods described herein is not more than about 10,000 ppm, preferably not more than about 2500 ppm, more preferably not more than about 400 ppm, more preferably not more than about 360 ppm, more preferably not more than about 320 ppm, more preferably not more than about 280 ppm, more preferably not more than about 240 ppm, more preferably not more than about 200 ppm, more preferably not more than about 160 ppm, more preferably not more than about 140 ppm, more preferably not more than about 120 ppm, more preferably not more than about 100 ppm, more preferably not more than about 80 ppm, more preferably not more than about 60 ppm, more preferably not more than about 40 ppm, more preferably not more than about 30 ppm, more preferably not more than about 20 ppm, more preferably not more than about 10 ppm, and most preferably not more than about 5 ppm. Such contamination can range from undetectable levels to about 10 ppm or from about 10 ppm to about 10,000 ppm. In some embodiments, a composition comprising a highly glycosylated protein (for example, recombinant human lubricin, for example, recombinant human lubricin produced or purified by a method described herein) described herein, the level of contaminating protein is (for example, host cell protein) is less than 1,000 ng/ mg highly glycosylated protein (ng/mg), less than 900 ng/mg, less than 800 ng/mg, less than 700 ng/mg, less than 600 ng/mg, less than 500 ng/mg, less than 400 ng/mg, less than 300 ng/mg, less than 200 ng/mg, or less than 100 ng/mg.

In certain embodiments, the invention provides a composition comprising purified recombinant lubricin, wherein the lubricin aggregate content is ≤2% (as determined, for example, by SE-HPLC (SEC) as described herein or any other such method known to those of skill in the art), the lubricin fragment content is ≤10% (determined, tor example, by SEC as described herein), the host cell protein content is ≤300 ng/mg as measured for example by ELISA, and the residual DNA content is ≤200,000 pg/mg as measured for example by qPCR.

In certain embodiments, the invention provides a composition comprising recombinant lubricin (for example, recombinant lubricin produced or purified by a method described herein), wherein the lubricin aggregate content is ≤2% (as determined, for example, by SE-HPLC (SEC) as described herein or any other such method known to those of skill in the art), the lubricin fragment content is ≤10% (determined, for example, by SEC as described herein), the host cell protein content is ≤300 ng/mg as measured for example by ELISA, and the residual DNA content is ≤200,000 pg/mg as measured for example by qPCR.

Host cell protein content of a composition comprising purified recombinant lubricin produced using a method described herein can be determined using any suitable method, for example, ELISA. In some embodiments, the invention provides a composition comprising purified recombinant lubricin, wherein the host cell protein content is ≤1,000 ng/mg of recombinant lubricin (ng/mg), ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg (e.g., ≤1,000 ng host cell protein/mg drug substance), as determined by ELISA. In some embodiments, the invention provides a composition comprising recombinant lubricin (for example, recombinant lubricin produced or purified by a method described herein), wherein the host cell protein content is ≤1,000 ng/mg, ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg (e.g., ≤1,000 ng host cell protein/mg drug substance), as determined by ELISA.

Contamination by residual host cell DNA (for example, CHO cell DNA) of a composition comprising purified recombinant lubricin can be determined by quantitative Polymerase Chain Reaction (qPCR) amplification of a repetitive sequence dispersed throughout the host cell genome. For example, the CHO cell genome includes a sequence of Alu-type repeats, of which about 300,000 copies are present per mammalian genome. These repeats can serve as surrogate markers for CHO DNA. Oligonucleotides serving as forward and reverse primers for amplification define a conserved 100 base-pair core region of this repetitive sequence. Total residual DNA in a sample can be determined by comparing the response generated by the contaminating DNA with that generated by a genomic reference standard, for example, a CHO genomic DNA reference standard, isolated from CHO KIPD parental cells. In some embodiments, the invention described herein provides a composition comprising purified recombinant lubricin, wherein the host cell residual DNA content is ≤300,000 pg/mg, ≤200,000 pg/mg, ≤100,000 pg/mg, ≤50,000 pg/mg, ≤10,000 pg/mg, <5,000 pg/mg, ≤1,000 pg/mg, ≤500 pg/mg, ≤100 pg/mg, ≤50 pg/mg, ≤10 pg/mg, or ≤5 pg/mg (e.g., ≤300,000 pg host cell DNA/mg drug substance), as determined by qPCR. In some embodiments, the invention described herein provides a composition comprising recombinant lubricin (for example, recombinant lubricin produced or purified by a method described herein), wherein the host cell residual DNA content is ≤300,000 pg/mg, ≤200,000 pg/mg, ≤100,000 pg/mg, ≤50,000 pg/mg, ≤10,000 pg/mg, ≤5,000 pg/mg, ≤1,000 pg/mg, ≤500 pg/mg, ≤100 pg/mg, ≤50 pg/mg, ≤10 pg/mg, or ≤5 pg/mg (e.g., ≤300,000 pg host cell DNA/mg drug substance), as determined by qPCR.

In certain embodiments, the invention provides a composition comprising purified recombinant lubricin, wherein bacterial endotoxin content of the composition is determined based on a bacterial endotoxin test (BET), for example, a limulus amebocyte lysate (LAL) test. Assays for determining bacterial endotoxin content can be performed in accordance with USP <85> and Ph. Eur. 2.6.14. For example, in some embodiments, the invention provides a composition comprising purified recombinant lubricin, wherein the bacterial endotoxin content is less than 8 endotoxin units (EU)/mL, less than 7 EU/mL, less than 6 EU/mL, less than 5 EU/mL, less than 4 EU/mL, less than 3 EU/mL, less than 2 EU/mL, less than 1

EU/mL, less than 0.9 EU/mL, less than 0.8 EU/mL, less than 0.7 EU/mL, less than 0.6 EU/mL, less than 0.5 EU/mL, less than 0.4 EU/mL, less than 0.3 EU/mL, less than 0.2 EU/mL, less than 0.1 EU/mL, less than 0.09 EU/mL, less than 0.08 EU/mL, less than 0.07 EU/mL, less than 0.06 EU/mL, less than 0.05 EU/mL, less than 0.04 EU/mL, less than 0.03 EU/mL, less than 0.02 EU/mL, or less than 0.01 EU/mL, as determined by BET. In some embodiments, the invention provides a composition comprising recombinant lubricin (for example, recombinant lubricin produced or purified by a method described herein), wherein the bacterial endotoxin content is less than 8 endotoxin units (EU)/mL, less than 7 EU/mL, less than 6 EU/mL, less than 5 EU/mL, less than 4 EU/mL, less than 3 EU/mL, less than 2 EU/mL, less than 1 EU/mL, less than 0.9 EU/mL, less than 0.8 EU/mL, less than 0.7 EU/mL, less than 0.6 EU/mL, less than 0.5 EU/mL, less than 0.4 EU/mL, less than 0.3 EU/mL, less than 0.2 EU/mL, less than 0.1 EU/mL, less than 0.09 EU/mL, less than 0.08 EU/mL, less than 0.07 EU/mL, less than 0.06 EU/mL, less than 0.05 EU/mL, less than 0.04 EU/mL, less than 0.03 EU/mL, less than 0.02 EU/mL, or less than 0.01 EU/mL, as determined by BET.

In certain embodiments, the invention provides a composition comprising purified recombinant lubricin, wherein microbial content of the composition is determined based on a microbial enumeration test (MET), for example, a total aerobic microbial count (TAMC) test or a total combined yeast/molds count (TYMC) test. MET can be performed in accordance with the microbiological methods of the Ph. Eur. chapters 2.6.12/2.6.13, USP chapters <61>/<62> and JP chapters <4.05> I/II. For example, in some embodiments, the invention provides a composition comprising purified recombinant lubricin, wherein the total aerobic microbial content is less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml, as determined by a TAMC test. In some embodiments, the invention provides a composition comprising recombinant lubricin (for example, recombinant lubricin produced or purified by a method described herein), wherein the total aerobic microbial content is less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml, as determined by a TAMC test. In some embodiments, the invention provides a composition comprising purified recombinant lubricin, wherein the total yeast and mold content is less than 1 CFU/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml, as determined by a TYMC test. In some embodiments, the invention provides a composition comprising recombinant lubricin (for example, recombinant lubricin produced or purified by a method described herein), wherein the total yeast and mold content is less than 1 CFU/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml, as determined by a TYMC test.

In certain embodiments, the invention provides a method of purifying a drug substance from a cell culture that is produced in a bioreactor that is at least about 1,000 L, at least about 1,500 L, at least about 2,000 L, at least about 2,500 L, or at least about 3,000 L in volume size. Thus, in some embodiments the invention provides a method of purifying a drug substance wherein the method includes culturing the cells in a bioreactor that is at least about 1,000 L, at least about 1,500 L, at least about 2,000 L, at least about 2,500 L, or at least about 3,000 L in volume size. After culturing cells in a bioreactor, the cells can be harvested. Such cell harvest is harvested, for example, by depth filtration followed by sterile filtration. The drug substance is then purified from the cell harvest by a method of the invention as described herein. In a particular embodiment, the drug substance is a heavily glycosylated recombinant protein, such as recombinant lubricin or other mucin-like protein or mucin protein.

In some embodiments, the methods described herein include one or more steps, wherein cells are pre-cultured in a volume smaller than that of a later bioreactor volume (for example, a bioreactor volume that is at least about 1,000 L, 1,500 L, 2,000 L, 2,500 L, or 3,000 L). For example, in some embodiments the method includes pre-culturing cells in a bioreactor volume of about 10 L, about 20 L, about 30 L, about 40 L, about 50 L, about 60 L, about 70 L, about 80 L, about 90 L, about 100 L, about 150 L, about 200 L, about 250 L, about 300 L, about 350 L, about 400 L, about 450 L, about 500 L, about 550 L, and/or about 600 L. For example, in some embodiments, a method described herein includes steps of pre-culturing cells in a bioreactor volume of about 10 L and pre-culturing cells in a bioreactor volume of about 92 L, before culturing the cells in a bioreactor volume of about 1,000 L. In some embodiments, a method described herein includes steps of pre-culturing cells in a bioreactor volume of about 20 L, pre-culturing cells in a bioreactor volume of about 100 L, and pre-culturing cells in a bioreactor volume of about 400 L, before culturing the cells in a bioreactor volume of about 2,000 L.

In certain embodiments, the invention provides a method of purifying at least about 1.5 g/L of a drug substance (e.g., about 1.5 g/L, 1.6 g/L, 1.7 g/L, 1.8 g/L, 1.9 g/L, 2.0 g/L, 2.1 g/L, 2.2 g/L., 2.3 g/L, 2.4 g/L, 2.5 g/L, 2.6 g/L, 2.7 g/L, 2.8 g/L, 2.9 g/L, about 3.0 g/L, from about 1.5 g/L to about 3.0 g/L, from about 1.5 g/L to about 3.5 g/L, from about 2.0 g/L to about 3.0 g/L, from about 1.5 g/L to about 2.5 g/L, from about 1.6 mg/ml to about 2.4 mg/ml, or from about 2.0 g/L to about 4.0 g/L) from a cell culture. In a particular embodiment, the drug substance is a heavily glycosylated recombinant protein, such as recombinant lubricin or other mucin-like protein or mucin protein. Thus, in some embodiments the invention provides a method of purifying an amount of heavily glycosylated protein from each unit of cell culture volume (for example, a method of purifying at least about 1.5 g of a heavily glycosylated protein from each liter of cell culture). In some embodiments, the amount of protein purified from a cell culture using a method described herein is determined by size exclusion chromatography (SEC). Thus, in some embodiments, the invention provides a method of purifying at least about 1.5 g/L of a drug substance (e.g., about 1.5 g/L, 1.6 g/L, 1.7 g/L, 1.8 g/L, 1.9 g/L, 2.0 g/L, 2.1 g/L, 2.2 g/L., 2.3 g/L, 2.4 g/L, 2.5 g/L, 2.6 g/L, 2.7 g/L, 2.8 g/L, 2.9 g/L, about 3.0 g/L, from about 1.5 g/L to about 3.0 g/L, from about 1.5 g/L to about 3.5 g/L, from about 2.0 g/L to about 3.0 g/L, from about 1.6 mg/ml to about 2.4 mg/ml, from about 1.5 g/L to about 2.5 g/L, or from about 2.0 g/L to about 4.0 g/L) from a cell culture, as determined by SEC. In some embodiments, the invention provides a method of purifying at least about 1.5 g/L of a drug substance (e.g., about 1.5 g/L, 1.6 g/L, 1.7 g/L, 1.8 g/L, 1.9 g/L, 2.0 g/L, 2.1 g/L, 2.2 g/L., 2.3 g/L, 2.4 g/L, 2.5 g/L, 2.6 g/L, 2.7 g/L, 2.8 g/L, 2.9 g/L, about 3.0 g/L, from about 1.5 g/L to about 3.0 g/L, from about 1.5 g/L to about 3.5 g/L, from about 1.6 mg/ml to about 2.4 mg/ml, from about 2.0 g/L to about 3.0 g/L, from about 1.5 g/L to about 2.5 g/L, or from about 2.0 g/L to about 4.0 g/L) from a cell culture, as determined by SEC, wherein the cell culture volume is at least about 1,000 L, at least about 1,500 L, at least about 2,000 L, at least about 2,500 L, at least about 3,000 L, about 1,000 L, about 1,500 L, about 2,000 L, about 2,500 L, or about 3,000 L.

### Potency

In some embodiments, the invention described herein provides methods that are effective to produce a composition comprising rhLubricin with specific potency characteristics. Potency of a composition described herein can be measured, for example, with a cell adhesion assay, for example, an A375 cell adhesion assay. For example, potency of a highly glycosylated protein can be determined based on its ability to inhibit the adhesion of A375 human melanoma cells to the surface of cell-tissue culture microtiter plates. If a highly glycosylated protein sample shows dose-dependent inhibition of adhesion of A375 cells in comparison to a reference substance, its identity can be confirmed. Thus, in some embodiments, a composition comprising a highly glycosylated protein purified using a method described herein shows a potency of between 50% and 150% (for example, 50%, 75%, 100%, 125%, or 150%) relative to biological activity of a reference substance, as determined by an A375 cell adhesion assay. In one aspect, disclosed herein is a method of determining potency of a composition comprising recombinant lubricin purified using a method described herein, wherein the method of determining potency comprises performing an A375 cell adhesion assay and measuring activity of the composition comprising recombinant lubricin relative to a reference standard. In some embodiments, the composition comprising recombinant lubricin shows activity of between 50% and 150% (for example, 50%, 75%, 100%, 125%, or 150%) relative to activity of a reference substance (for example, a reference sample of purified recombinant lubricin), as determined by the A375 cell adhesion assay.

In some embodiments, the invention described herein provides methods that are effective to produce a composition comprising rhLubricin with specific potency characteristics. Potency of a composition described herein can be measured, for example, with a reporter cell assay, for example, an NF-κB reporter cell assay. For example, potency of a highly glycosylated protein can be determined based on its ability to increase NF-κB-mediated reporter gene (e.g., lucifersase or Lucia™) expression. If a highly glycosylated protein sample shows a dose-dependent increase in NF-κB-mediated reporter gene expression in comparison to a reference substance, its identity can be confirmed. Thus, in some embodiments, a composition comprising a highly glycosylated protein produced or purified using a method described herein shows a potency of between 50% and 150% (for example, 50%, 75%, 100%, 125%, or 150%) relative to biological activity of a reference substance, as determined by a reporter cell assay, for example, an NF-κB reporter cell assay. In one aspect, disclosed herein is a method of determining potency of a composition comprising recombinant lubricin produced or purified using a method described herein, wherein the method of determining potency comprises performing an NF-κB reporter cell assay and measuring activity (for example, as determined by reporter gene expression) of the composition comprising recombinant lubricin relative to a reference standard. In some embodiments, the composition comprising recombinant lubricin shows activity of between 50% and 150% (for example, 50%, 75%, 100%, 125%, or 150%) relative to activity of a reference substance (for example, a reference sample of purified recombinant lubricin), as determined by the NF-κB reporter cell assay.

Potency of a composition described herein can be measured, for example, with a cell surface protein binding assay, for example, a cell surface receptor cluster determinant 44 (CD44) binding assay. For example, potency of a highly glycosylated protein can be determined based on its ability to compete for binding to CD44, as measured by ELISA and surface plasmon resonance (for example, as described in Al-Sharif et al., (2015) “Lubricin/Proteoglycan 4 Binding to CD44 Receptor: A Mechanism of Lubricin's suppression of Pro-inflammatory Cytokine Induced Synoviocyte Proliferation,” Arthritis Rheumatol. 67(6):1503-13). If a highly glycosylated protein sample shows competitive binding to CD44 in comparison to a reference substance, its identity can be confirmed. Thus, in some embodiments, a composition comprising a highly glycosylated protein purified using a method described herein shows a potency of between 50% and 150% (for example, 50%, 75%, 100%, 125%, or 150%) relative to biological activity of a reference substance, as determined by a CD44 binding assay. In one aspect, disclosed herein is a method of determining potency of a composition comprising recombinant lubricin purified using a method described herein, wherein the method of determining potency comprises performing a CD44 binding assay and measuring activity of the composition comprising recombinant lubricin relative to a reference standard. In some embodiments, the composition comprising recombinant lubricin shows activity of between 50% and 150% (for example, 50%, 75%, 100%, 125%, or 150%) relative to activity of a reference substance (for example, a reference sample of purified recombinant lubricin), as determined by the CD44 binding assay.

### Stability

In some embodiments, the invention described herein provides methods that are effective to produce a composition comprising rhLubricin with particular stability characteristics. In particular, the methods described herein are effective to produce a stable composition comprising rhLubricin composition wherein less than or equal to about 15% of the rhLubricin of the composition undergo fragmentation over a given period of time at a given temperature. As used herein, a “stable composition of rhLubricin” or a “composition of rhLubricin that is stable” refers to a composition comprising rhLubricin wherein 15% or less of rhLubricin of the initial composition undergoes fragmentation over a given period of time at a given temperature. For example, in some embodiments, the methods described herein are effective to produce a stable composition of rhLubricin wherein less than or equal to about 5%, 6%, 7%, 8%, 9%, 9.5%, 10%, 11%, 12%, 13%, 14%, or 15% of the rhLubricin of the initial composition undergoes fragmentation over a given period of time at a given temperature. Fragmentation of rhLubricin can be measured using methods known in the art, for example, size exclusion chromatography assays or reducing capillary electrophoresis under denaturing conditions (rCE-SDS).

Thus, in some embodiments, a method described herein is effective to produce a composition of rhLubricin that is stable at about 5° C. or lower for about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, from about 12 months to about 24 months, from about 14 months to about 24 months, from about 16 months to about 24 months, from about 18 months to about 24 months, from about 20 months to about 24 months, or from about 18 months to about 26 months. In some embodiments, a method described herein is effective to produce a composition of rhLubricin that is stable at about 25° C. or lower for about 1 day, about 3 days, about 5 days, about 1 week, about 2 weeks, about 3 weeks, about 4 weeks, about 1 month, about 2 months, about 3 months, about 4 months, about 5 months, about 6 months, from about 1 week to 1 month, from about 2 weeks to 1 month, from about 3 weeks to 1 month, or from about 1 month to 2 months. In some embodiments, a method described herein is effective to produce a composition of rhLubricin that is stable at about 40° C. or lower for about 1 day, about 3 days, about 5 days, about 1 week, about 2 weeks, about 3 weeks, about 4 weeks, about 1 month, from about 1 week to 1 month, from about 2 weeks to 1 month, or from about 3 weeks to 1 month. In a particular embodiment, a method described herein is effective to produce a composition of rhLubricin that is stable at 5° C. for 24 months. In some embodiments, a method described herein is effective to produce a composition of rhLubricin that is stable at 25° C. for 1 month. In some embodiments, the stable composition of rhLubricin has an initial concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, or between about 0.15 mg/ml and about 0.45 mg/ml.

Additionally, described herein is a stable composition of rhLubricin produced using a method described herein that is stable at about 5° C. or lower for about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, from about 12 months to about 24 months, from about 14 months to about 24 months, from about 16 months to about 24 months, from about 18 months to about 24 months, from about 20 months to about 24 months, or from about 18 months to about 26 months. Also described herein is a composition of rhLubricin produced using a method described herein that is stable at about 25° C. or lower for about 1 day, about 3 days, about 5 days, about 1 week, about 2 weeks, about 3 weeks, about 4 weeks, about 1 month, about 2 months, about 3 months, about 4 months, about 5 months, about 6 months, from about 1 week to 1 month, from about 2 weeks to 1 month, from about 3 weeks to 1 month, or from about 1 month to 2 months. Also described herein is a composition of rhLubricin produced using a method described herein that is stable at about 40° C. or lower for about 1 day, about 3 days, about 5 days, about 1 week, about 2 weeks, about 3 weeks, about 4 weeks, about 1 month, from about 1 week to 1 month, from about 2 weeks to 1 month, or from about 3 weeks to 1 month. In a particular embodiment, described herein is a composition of rhLubricin produced using a method described herein that is stable at 5° C. for 24 months. In some embodiments, described herein is a composition of rhLubricin produced using a method described herein that is stable at 25° C. for 1 month. In some embodiments, the stable composition of rhLubricin produced using a method described herein has an initial concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, or between about 0.15 mg/ml and about 0.45 mg/ml.

### Final Buffer Solution

In some embodiments, an rhLubricin composition described herein is formulated in final buffer solution, for example, after completion of all rhLubricin purification steps. Such a final buffer solution is suitable for administration to a subject, for example, a human subject. In some embodiments, an rhLubricin composition described herein is formulated in final buffer solution comprising sodium phosphate, sodium chloride, and polysorbate (for example polysorbate 20). For example, in some embodiments, an rhLubricin composition described herein is formulated in final buffer solution comprising 10 mM sodium phosphate, 140 mM sodium choloride, and 0.02% (w/v) polysorbate 20. In some embodiments, an rhLubricin composition described herein is formulated in final buffer solution comprising sodium phosphate (for example, 5m M, 10 mM, 15, mM, 20 mM, 25 mM, 5 mM to 10 mM, 5 mM to 15 mM, 10 mM to 15 mM, or 10-20 mM sodium phosphate), sodium choloride (for example, 100 mM, 110 mM, 120 mM, 130 mM, 140 mM, 150 mM, 160 mM, 100 mM to 120 mM, 120 mM to 140 mM, 130 mM to 150 mM, or 140 mM to 150 mM sodium chloride), and a detergent (for example, 0.01%, 0.02%, 0.03%, 0.04%, 0.05%, 0.10%, 0.01% to 0.10%, 0.01% to 0.03%, 0.01% to 0.05%, 0.02% to 0.04%, or 0.02% to 0.05% (w/v) detergent, for example, polysorbate 20). In some embodiments, described herein is a method that includes the step of dissolving an rhLubricin composition in a final buffer solution comprising 10 mM sodium phosphate, 140 mM sodium choloride, and 0.02% (w/v) polysorbate 20.

In some embodiments described herein, an rhLubricin composition comprising rhLubricin purified using a method described herein and formulated in a final buffer solution comprising sodium phosphate, sodium chloride, and polysorbate (for example polysorbate 20), is stable at about 5° C. or lower for about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, from about 12 months to about 24 months, from about 14 months to about 24 months, from about 16 months to about 24 months, from about 18 months to about 24 months, from about 20 months to about 24 months, or from about 18 months to about 26 months. In some embodiments, an rhLubricin composition comprising rhLubricin purified using a method described herein and formulated in a final buffer solution comprising sodium phosphate, sodium chloride, and polysorbate (for example polysorbate 20), is stable at about 25° C. or lower for about 1 day, about 3 days, about 5 days, about 1 week, about 2 weeks, about 3 weeks, about 4 weeks, about 1 month, about 2 months, about 3 months, about 4 months, about 5 months, about 6 months, from about 1 week to 1 month, from about 2 weeks to 1 month, from about 3 weeks to 1 month, or from about 1 month to 2 months. In some embodiments, an rhLubricin composition comprising rhLubricin purified using a method described herein and formulated in a final buffer solution comprising sodium phosphate, sodium chloride, and polysorbate (for example polysorbate 20), is stable at about 40° C. or lower for about 1 day, about 3 days, about 5 days, about 1 week, about 2 weeks, about 3 weeks, about 4 weeks, about 1 month, from about 1 week to 1 month, from about 2 weeks to 1 month, or from about 3 weeks to 1 month. In a particular embodiment, an rhLubricin composition comprising rhLubricin purified using a method described herein and formulated in a final buffer solution comprising sodium phosphate, sodium chloride, and polysorbate (for example polysorbate 20), is stable at 5° C. for 24 months. In some embodiments, an rhLubricin composition comprising rhLubricin purified using a method described herein and formulated in a final buffer solution comprising sodium phosphate, sodium chloride, and polysorbate (for example polysorbate 20), is stable at 25° C. for 1 month. In some embodiments, a composition of rhLubricin described herein has an initial concentration of about 0.15 mg/ml, about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, or between about 0.15 mg/ml and about 0.45 mg/ml.

pH of a final buffer solution or a final solution comprising a highly glycosylated protein can be measured according to protocols, for example, USP <791>and Ph. Eur. 2.2.3. In some embodiments, the final buffer solution has a pH of about 7.0. In some embodiments, the final buffer solution has a pH of about 6.9. In some embodiments, the final buffer solution has a pH of between about 6.5 and about 7.5, between about 6.6 and about 7.4, between about 6.7 and about 7.3, between about 6.8 and about 7.2, between about 6.9 and about 7.1, or between about 6.9 and about 7.0. For example, in some embodiments, the final buffer solution has a pH of about 6.5, about 6.6, about 6.7, about 6.8, about 6.9, about 7.0, about 7.1, about 7.2, about 7.3, about 7.4, or about 7.5.

In some embodiments, an rhLubricin composition described herein is freeze dried. In some embodiments, an rhLubricin composition described herein is freeze dried and stored at a suitable temperature, for example, below −20° C. or below −60° C. (for example, −80° C.). In some embodiments, a method of purifying a recombinantly produced glycosylated polypeptide, for example, a recombinantly produced glycosylated lubricin protein, described herein includes a step of freeze-drying a composition comprising recombinantly produced glycosylated polypeptide (for example, recombinantly produced glycosylated lubricin protein) purified using a method described herein. Thus, in some embodiments, the invention relates to a method of purifying a recombinantly produced glycosylated polypeptide, in particular a recombinantly produced glycosylated lubricin, wherein the method comprises three successive chromatography steps: (a) a first chromatography step consisting of multimodal cation exchange chromatography (MCC); (b) a second chromatography step consisting of multimodal anion exchange chromatography (MAC); and (c) a third chromatography step consisting of hydrophobic interaction chromatography (HIC); and (ii) further comprises a step of freeze-drying the recombinantly produced glycosylated polypeptide. In some embodiments, the method further comprises a step of depth filtration. In some embodiments, the step of depth filtration is performed prior to the HIC step. In some embodiments, depth filtration is performed using a suitable filter, for example, a cellulose or polypropylene fiber-based filter, for example, a positively charged triple layer B1HC filter.

Further non-limiting embodiments of the present disclosure are described in the following embodiments (the following embodiments are also applicable to glycoproteins (especially proteins having at least about 25% or more glycosylation) other than lubricin, as discussed and otherwise provided herein):

1. A method of purifying a recombinant lubricin glycoprotein, comprising the steps of subjecting a cell culture harvest containing said lubricin glycoprotein to: a multimodal cation exchange chromatography (MCC), a multimodal anion exchange chromatography (MAC), and a hydrophobic interaction chromatography (HIC), which are performed in any order.

2. The method of embodiment 1, wherein the steps are performed in the following order: a) MCC, b) MAC, and c) HIC.

3. The method of embodiment 2, further comprising contacting cells in culture with MgCl₂ and an endonuclease, and harvesting the cells to obtain said cell culture harvest, prior to step a).

4. The method of embodiment 3, wherein said cells in culture are in a culture volume of about 1,000 L, about 1,500 L, about 2,000 L, or about 2,500 L.

5. The method of embodiment 2, further comprising contacting the cell culture harvest with MgCl₂ and an endonuclease prior to step a).

6. The method of embodiment 5, wherein the cell culture harvest is from a cell culture volume of about 1,000 L, about 1,500 L, about 2,000 L, or about 2,500 L.

7. The method of embodiment 3 or 5, wherein the endonuclease is Benzonase® endonuclease.

8. The method of embodiment 5, further comprising cooling the cell culture harvest to 2-8° C. before said contacting with MgCl₂ and the endonuclease.

9. The method of any one of the preceding embodiments, further comprising a step of virus inactivation after the multimodal anion exchange chromatography (MAC) step and before the hydrophobic interaction chromatography (HIC) step.

10. The method of embodiment 9, wherein the virus inactivation step comprises adjusting the pH of the solution obtained from step b) to about 3.4-3.6.

11. The method of embodiment 10, wherein after incubating the solution for at least one hour, adjusting the pH to about 7.0 before the hydrophobic interaction chromatography (HIC) step.

12. The method of any one of embodiments 9-11, further comprising a depth filtration step prior to the hydrophobic interaction chromatography (HIC) step.

13. The method of embodiment 12, wherein the depth filtration step follows the virus inactivation step.

14. The method of any one of the preceding embodiments, comprising a virus removal step after the hydrophobic interaction chromatography (HIC) step.

15. The method of embodiment 14, wherein the virus removal step comprises nanofiltration.

16. The method of embodiment 14 or 15, further comprising an ultrafiltration step after the virus removal step.

17. The method of any one of embodiments 14-16, comprising a second virus inactivation step after the virus removal step.

18. The method of embodiment 17, wherein the second virus inactivation step comprises adding a dimethylurea solution.

19. The method of embodiment 17 or 18, comprising an ultrafiltration step after the second virus inactivation step.

20. The method of embodiment 1 or 2, further comprising one or more ultrafiltration and/or nanofiltration steps.

21. The method of embodiment 1 or 2, further comprising one or more virus inactivation steps.

22. The method of embodiment 1 or 2, further comprising one or more virus removal steps.

23. The method of any one of the preceding embodiments, wherein of the recombinant lubricin glycoprotein comprises the amino acid sequence of amino acid residues 25-1404 of SEQ ID NO:1 or 2.

24. The method of any one of the preceding embodiments, wherein at least 30% of the molecular weight of the recombinant lubricin glycoprotein is from glycosidic residues.

25. The method of any one of the preceding embodiments, wherein at least 90% of O-glycosylation of the lubricin glycoprotein is core 1 glycosylation.

26. The method of any one of the preceding embodiments, wherein the lubricin glycoprotein comprises O-glycan species, wherein the O-glycan species comprise about 7% or more Gal-GalNAc, about 80% or more 2,3-NeuAc Core 1, about 3% or more 2*NeuAc Core 1, and about 1% or more 2,3-NeuGc Core 1.

27. The method of any one of the preceding embodiments, wherein the lubricin glycoprotein comprises about 50 μg or more NANA per mg of the lubricin glycoprotein.

28. The method of any one of the preceding embodiments, wherein the lubricin glycoprotein comprises about 10 μg or less NGNA per mg of the lubricin glycoprotein.

29. The method of any one of the preceding embodiments, wherein the lubricin glycoprotein comprises about 100 μg or more Gal per mg of the lubricin glycoprotein.

30. The method of any one of the preceding embodiments, wherein the lubricin glycoprotein comprises about 100 μg or more GalNAc per mg of the lubricin glycoprotein.

31. A recombinant lubricin glycoprotein obtained by the method according to any one of the preceding embodiments.

32. A pharmaceutical composition comprising the recombinant lubricin glycoprotein according to embodiment 31 and a pharmaceutically acceptable excipient.

33. The pharmaceutical composition of embodiment 32, wherein purity of the pharmaceutical composition is 95%, 96%, 97%, 98%, 99%, or greater, as determined by reversed phase chromatography (RPC).

34. The pharmaceutical composition of embodiment 32 or 33, comprising less than 1% of aggregates of the recombinant lubricin glycoprotein.

35. The pharmaceutical composition of any one of embodiments 32-34, comprising less than 1% of fragments of the recombinant lubricin glycoprotein.

36. The pharmaceutical composition of any one of embodiments 32-35, comprising ≤1,000 ng host cell protein/mg of recombinant lubricin glycoprotein (ng/mg), 900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg.

37. The pharmaceutical composition of any one of embodiments 32-36, comprising ≤10,000 pg host cell DNA/mg of recombinant lubricin glycoprotein (pg/mg), 5,000 pg/mg, ≤1,000 pg/mg, ≤500 pg/mg, ≤100 pg/mg, ≤50 pg/mg, ≤10 pg/mg, or ≤5 pg/mg.

38. The pharmaceutical composition of any one of embodiments 32-37, comprising less than 8 endotoxin units (EU)/mL, less than 1 EU/mL, less than 0.1 EU/mL, or less than 0.01 EU/mL, as determined by a bacterial endotoxin test (BET).

39. The pharmaceutical composition of any one of embodiments 32-38, having a total aerobic microbial count (TAMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

40. The pharmaceutical composition of any one of embodiments 32-39, having a total combined yeast/mold count (TYMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

41. The pharmaceutical composition of any one of embodiments 32-40, wherein the composition is stable at 5° C. for at least 24 months.

42. The pharmaceutical composition of any one of embodiments 32-41, wherein the composition is stable at 25° C. for at least 1 month.

43. The pharmaceutical composition of any one of embodiments 32-42, wherein the composition has an initial concentration of about 0.15 mg recombinant lubricin glycoprotein/ml (mg/ml), about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, about 0.70 mg/ml, about 0.8 mg/ml, about 0.9 mg/ml, about 1.0 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/mi., about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.5 mg/ml, about 4.0 mg/ml, from about 0.15 mg/ml to about 3.0 mg/ml, from about 0.45 mg/ml to about 3.0 mg/ml, from about 0.15 mg/ml to about 0.45 mg/ml, from about 1.5 mg/ml to about 3.0 mg/ml, from about 1.5 mg/ml to about 3.5 mg/ml, from about 2.0 mg/ml to about 3.0 mg/ml, from about 1.5 mg/ml to about 2.5 mg/ml, or from about 2.0 mg/ml to about 4.0 mg/ml.

44. A method for treating an ocular surface disorder, comprising a step of administering the pharmaceutical composition of any one of embodiments 32-43 to a patient.

45. The method of embodiment 44, wherein the ocular surface disorder is dry eye disease.

46. A method of producing a recombinant lubricin glycoprotein comprising the steps of:

a) generating a Chinese Hamster Ovary (CHO) cell clone which produces the recombinant lubricin glycoprotein,

b) cultivating of the CHO host cells under suitable conditions, thereby obtaining a cell culture containing a recombinant lubricin glycoprotein, and

c) purifying the recombinant lubricin glycoprotein from the cell culture according to the method of any one of embodiments 1 to 30.

47. A method of producing a recombinant lubricin glycoprotein comprising the steps of:

a) cultivating under suitable conditions mammalian host cells that comprise a nucleic acid molecule that encodes a lubricin glycoprotein; and

b) purifying the recombinant lubricin glycoprotein from the cell culture according to the method of any one of embodiments 1 to 30.

48. The method of embodiment 47, wherein the mammalian host cells are Chinese

Hamster Ovary (CHO) cells.

49. The method of embodiment 48, wherein the CHO cells are CHO-M cells.

50. A process for production of a purified recombinant human lubricin glycoprotein, the process comprising steps of:

i) culturing a mammalian cell capable of producing recombinant human Lubricin (rhLubricin) into a liquid medium; and

ii) concentrating, purifying and formulating the rhLubricin by a purification process comprising one or more steps of: Multimodal Cation exchange Chromatography (MCC), multimodal anion exchange chromatography (MAC), and/or hydrophobic interaction chromatography (HIC),

wherein the rhLubricin produced is selected from the group consisting of: (a) amino acids 25-1404 of the amino acid sequence of SEQ ID NO: 1 or 2; (b) a functionally equivalent variant of rhLubricin having an amino acid sequence that is at least 75 percent identical to amino acids 25-1404 of the sequence of SEQ ID NO: 1, which has substantially the same activity as full-length and naturally occurring lubricin; and (c) a functionally equivalent lubricin fragment comprising glycosylated repeats of SEQ ID NO: 3.

51. The process according to embodiment 50, comprising adding an endonuclease to the liquid medium before applying the liquid medium to a Multimodal Cation exchange Chromatography (MCC).

52. The process according to embodiment 50 or 51, wherein the MCC, MAC, and

HIC steps are successive.

53. The process according to any one of embodiments 50-52, wherein the recovery yield of rhLubricin after the MCC chromatography step is about 45-75%.

54. The process according to embodiment 52, wherein the recovery yield of rhLubricin after the MAC chromatography step is about 80-90%.

55. The process according to embodiment 52, wherein the recovery yield of rhLubricin after the HIC chromatography step is about 93-100%.

56. The process according to any one of embodiments 50 to 55, comprising at least one virus inactivation step.

57. The process according to embodiment 56, wherein at least one virus inactivation step comprises adjusting the pH of the eluate from a chromatography step to a pH of about 3.4-3.6.

58. The process according to embodiment 56, wherein at least one virus inactivation step comprises incubating the eluate from a chromatography step with dimethylurea.

59. The process according to any embodiments 50 to 58, comprising two virus inactivation steps.

60. The process according to embodiment 56, wherein at least one virus inactivation step comprises adjusting the pH of the eluate from a chromatography step to pH 3.5, and the second virus inactivation step comprises incubating the eluate from a separate chromatography step with dimethylurea.

61. The process according to embodiment 60, wherein the chromatography step before the first virus inactivation step is MAC.

62. The process according to embodiment 60 or 61, wherein the chromatography step before the second virus inactivation step is HIC.

63. The process according to any one of embodiments 50-58, comprising a depth filtration step prior to the hydrophobic interaction chromatography (HIC) step.

64. The process according to embodiment 63, wherein the depth filtration step follows the at least one virus inactivation step.

65. The process according to any one of embodiments 50 to 64, comprising subjecting a liquid solution from the HIC step to nanofiltration.

66. The process according to embodiment 65, wherein the nanofiltration occurs before virus inactivation.

67. The process according to any one of embodiments 50 to 64, comprising subjecting a liquid solution from a chromatography step to ultrafiltration and compounding 68. The process according to any one of embodiments 56 to 67, wherein the recovery yield of rhLubricin after the first virus inactivation step is about 90-99%.

69. The process according to any one of embodiments 56 to 68, wherein the recovery yield of rhLubricin after the second virus inactivation step is about 95-99%.

70. The process according to any one of embodiments 67 to 69, wherein the recovery yield of rhLubricin after the ultrafiltration and compounding step is about 92-95%.

71. The process according to any one of embodiments 50-70, wherein step i) further comprises treating the mammalian cell with an endonuclease.

72. The process according to any of embodiments 50 to 70, wherein step ii) comprises the following steps:

II) introducing a supernatant containing rhLubricin into an equilibrated chromatography column and eluting one or more fraction(s) containing rhLubricin into a solution;

III) polishing the rhLubricin containing solution from step II in one or two or more successive steps, each step comprising loading the preparation on an equilibrated chromatography column(s) and eluting one or more fraction(s) containing rhLubricin;

IV) subjecting the rhLubricin containing solution from step 111 to virus inactivation;

V) polishing the rhLubricin containing solution from step IV in one or two or more successive steps, each step comprising loading the preparation on an equilibrated chromatography column(s) and eluting one or more fraction(s) containing rhLubricin;

VI) passing the fraction(s) from step V through a viral reduction filter and/or inactivating virus in said fraction(s) with a virus inactivating agent; and

VII) formulating the fraction(s) from step VI in order to obtain a preparation of rhLubricin in a suitable formulation buffer.

73. The process according to embodiment 72, further comprising an initial step I of treating the supernatant containing rhLubricin with an endonuclease.

74. The process according to any of embodiments 72 or 73, wherein the chromatography column used in step II of the purification process is a cation exchange column.

75. The process according to embodiment 74, wherein said anion exchange column is a multimodal cation exchange chromatography (MCC) column.

76. The process according to any of embodiments 72 to 75, wherein the chromatography column used in step III of the purification process is an anion exchange column.

77. The process according to embodiment 76, wherein the chromatography column is a multimodal anion-exchange chromatography (MAC) column.

78. The process according to any of embodiments 72 to 77, wherein the chromatography column used in step V of the purification process is hydrophobic interaction column.

79. The process according to any of embodiments 72 to 78, wherein the filtration of the sample as performed in step VI of the purification process is replaced by or combined with contacting the sample with a detergent.

80. The process according to any of embodiments 72 to 78, wherein the filtration of the sample as performed in step VI of the purification process is replaced by or combined with contacting the sample with dimethylurea.

81. The process according to any of embodiments 72 to 80, wherein the virus inactivating agent is a detergent or dimethylurea.

82. The process according to any of embodiments 72 to 81, further comprising a step VIII) of filling the formulated preparation of rhLubricin into a suitable container and freeze-drying the sample.

83. The process according to any of embodiments 72 to 81, further comprising a step of subjecting the fraction(s) from step VI to ultrafiltration/diafiltration.

84. The process according to embodiment 83, further comprising a step VIII) of filling the formulated preparation of rhLubricin into a suitable container and freeze-drying the sample.

85. The process according to any one of embodiments 50 to 84, wherein at least 30% of the molecular weight of the rhLubricin is from glycosidic residues.

86. The process according to any one of embodiments 50 to 85, wherein at least 90% of O-glycosylation of the rhLubricin is core 1 glycosylation.

87. The process according to any one of embodiments 50 to 86, wherein the rhLubricin comprises O-glycan species, wherein the O-glycan species comprise about 7% or more Gal-GalNAc, about 80% or more 2,3-NeuAc Core 1, about 3% or more 2*NeuAc Core 1, and about 1% or more 2,3-NeuGc Core 1.

88. The process according to any one of embodiments 50 to 87, wherein the rhLubricin comprises about 50 μg or more NANA per mg of the lubricin glycoprotein.

89. The process according to any one of embodiments 50 to 88, wherein the rhLubricin comprises about 10 μg or less NGNA per mg of the lubricin glycoprotein.

90. The process according to any one of embodiments 50 to 89, wherein the rhLubricin comprises about 100 μg or more Gal per mg of the lubricin glycoprotein.

91. The process according to any one of embodiments 50 to 90, wherein the rhLubricin comprises about 100 μg or more GalNAc per mg of the lubricin glycoprotein.

92. The process according to any of embodiments 50 to 84, wherein said rhLubricin is combined with a pharmaceutically acceptable carrier.

93. A composition comprising rhLubricin that is purified according to the process of any one of embodiments 50 to 92.

94. The composition of embodiment 93, wherein purity of the composition is 95%, 96%, 97%, 98%, 99%, or greater, as determined by reversed phase chromatography (RPC).

95. The composition of embodiment 93 or 94, comprising less than 1% of aggregates of the rhLubricin.

96. The composition of any one of embodiments 93-95, comprising less than 1% of fragments of the rhLubricin.

97. The composition of any one of embodiments 93-96, comprising ≤1,000 ng host cell protein/mg of rhLubricin (ng/mg), ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg.

98. The composition of any one of embodiments 93-97, comprising ≤10,000 pg host cell DNA/mg of rhLubricin (pg/mg), ≤5,000 pg/mg, ≤1,000 pg/mg, ≤500 pg/mg, ≤100 pg/mg, ≤50 pg/mg, ≤10 pg/mg, or ≤5 pg/mg.

99. The composition of any one of embodiments 93-98, comprising less than 8 endotoxin units (EU)/mL, less than 1 EU/mL, less than 0.1 EU/mL, or less than 0.01 EU/mL, as determined by a bacterial endotoxin test (BET).

100. The composition of any one of embodiments 93-99, having a total aerobic microbial count (TAMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

101. The composition of any one of embodiments 93-100, having a total combined yeast/mold count (TYMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

102. The composition of any one of embodiments 93-101, wherein the composition is stable at 5° C. for at least 24 months.

103. The composition of any one of embodiments 93-102, wherein the composition is stable at 25° C. for at least 1 month.

104. The composition of any one of embodiments 93-103, wherein the composition has an initial concentration of about 0.15 mg recombinant lubricin glycoprotein/ml (mg/ml), about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, about 0.70 mg/ml, about 0.8 mg/ml, about 0.9 mg/ml, about 1.0 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml., about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.5 mg/ml, about 4.0 mg/ml, from about 0.15 mg/ml to about 3.0 mg/ml, from about 0.45 mg/ml to about 3.0 mg/ml, from about 0.15 mg/ml to about 0.45 mg/ml, from about 1.5 mg/ml to about 3.0 mg/ml, from about 1.5 mg/ml to about 3.5 mg/ml, from about 2.0 mg/ml to about 3.0 mg/ml, from about 1.5 mg/ml to about 2.5 mg/ml, or from about 2.0 mg/ml to about 4.0 mg/ml.

105. A pharmaceutical composition comprising a recombinant lubricin glycoprotein and a pharmaceutically acceptable carrier, wherein purity of the composition is 95%, 96%, 97%, 98%, 99%, or greater, as determined by reversed phase chromatography (RPC),

wherein the composition comprises less than 1% of aggregates of the recombinant lubricin glycoprotein,

wherein the composition comprises less than 1% of fragments of the recombinant lubricin glycoprotein, wherein the composition comprises ≤1,000 ng host cell protein/mg of recombinant lubricin glycoprotein (ng/mg), ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg,

wherein the composition comprises ≤10,000 pg host cell DNA/mg of recombinant lubricin glycoprotein (pg/mg), ≤5,000 pg/mg, ≤1,000 pg/mg, ≤500 pg/mg, ≤100 pg/mg, ≤50 pg/mg, ≤10 pg/mg, or ≤5 pg/mg,

wherein the composition comprises less than 8 endotoxin units (EU)/mL, less than 1 EU/mL, less than 0.1 EU/mL, or less than 0.01 EU/mL, as determined by a bacterial endotoxin test (BET),

wherein the composition has a total aerobic microbial count (TAMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml, and/or

wherein the composition has a total combined yeast/mold count (TYMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

106. The pharmaceutical composition of embodiment 105, wherein the composition is stable at 5° C. for at least 24 months.

107. The pharmaceutical composition of embodiment 105, wherein the composition is stable at 25° C. for at least 1 month.

108. The pharmaceutical composition of any one of embodiments 105-107, wherein the composition has an initial concentration of about 0.15 mg recombinant lubricin glycoprotein/ml (mg/ml), about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, about 0.70 mg/ml, about 0.8 mg/ml, about 0.9 mg/ml, about 1.0 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/mi., about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.5 mg/ml, about 4.0 mg/ml, from about 0.15 mg/ml to about 3.0 mg/ml, from about 0.45 mg/ml to about 3.0 mg/ml, from about 0.15 mg/ml to about 0.45 mg/ml, from about 1.5 mg/ml to about 3.0 mg/ml, from about 1.5 mg/ml to about 3.5 mg/ml, from about 2.0 mg/ml to about 3.0 mg/ml, from about 1.5 mg/ml to about 2.5 mg/ml, or from about 2.0 mg/ml to about 4.0 mg/ml.

109. The pharmaceutical composition of any one of embodiments 105-108, wherein at least 30% of the molecular weight of the recombinant lubricin glycoprotein is from glycosidic residues.

110. The pharmaceutical composition of any one of embodiments 105-109, wherein at least 90% of O-glycosylation of the recombinant lubricin glycoprotein is core 1 glycosylation.

111. The pharmaceutical composition of any one of embodiments 105-110, wherein the recombinant lubricin glycoprotein comprises O-glycan species, wherein the O-glycan species comprise about 7% or more Gal-GalNAc, about 80% or more 2,3-NeuAc Core 1, about 3% or more 2*NeuAc Core 1, and about 1% or more 2,3-NeuGc Core 1.

112. The pharmaceutical composition of any one of embodiments 105-111, wherein the recombinant lubricin glycoprotein comprises about 50 μg or more NANA per mg of the lubricin glycoprotein.

113. The pharmaceutical composition of any one of embodiments 105-112, wherein the recombinant lubricin glycoprotein comprises about 10 μg or less NGNA per mg of the lubricin glycoprotein.

114. The pharmaceutical composition of any one of embodiments 105-113, wherein the recombinant lubricin glycoprotein comprises about 100 μg or more Gal per mg of the lubricin glycoprotein.

115. The pharmaceutical composition of any one of embodiments 105-114, wherein the recombinant lubricin glycoprotein comprises about 100 μg or more GalNAc per mg of the lubricin glycoprotein.

116. The pharmaceutical composition of any one of embodiments 105-115, wherein the recombinant lubricin glycoprotein is selected from the group consisting of: (a) amino acids 25-1404 of the amino acid sequence of SEQ ID NO: 1 or 2; (b) a functionally equivalent variant of recombinant lubricin glycoprotein having an amino acid sequence that is at least 75 percent identical to amino acids 25-1404 of the sequence of SEQ ID NO: 1, which has substantially the same activity as full-length and naturally occurring lubricin; and (c) a functionally equivalent lubricin fragment comprising glycosylated repeats of SEQ ID NO: 3, or a mixture thereof.

117. The pharmaceutical composition of any one of embodiments 105-116, wherein the recombinant lubricin glycoprotein is purified according to the method of any one of embodiments 1-20.

118. The pharmaceutical composition of any one of embodiments 105-116, wherein the recombinant lubricin glycoprotein is produced according to the method of any one of embodiments 46-49.

119. The pharmaceutical composition of any one of embodiments 105-116, wherein the recombinant lubricin glycoprotein is produced according to the process of any one of embodiments 50-84 or 92.

120. A method of treating an ocular surface disease, comprising administering the pharmaceutical composition of any one of embodiments 105-119 to a patient in need thereof. 121. The method of embodiment 120, wherein the disease is dry eye disease.

122. A method of producing a recombinant lubricin glycoprotein, comprising the steps of subjecting a cell culture harvest containing said lubricin glycoprotein to: a multimodal cation exchange chromatography (MCC), a multimodal anion exchange chromatography (MAC), and a hydrophobic interaction chromatography (HIC), which are performed in any order.

123. The method of embodiment 122, wherein the steps are performed in the following order: a) MCC, b) MAC, and c) HIC.

124. The method of embodiment 123, wherein prior to step a), contacting cells in culture with MgCl₂ and an endonuclease, and harvesting the cells to obtain said cell culture harvest.

125. The method of embodiment 124, wherein said cells in culture are in a culture volume of about 1,000 L, about 1,500 L, about 2,000 L, or about 2,500 L.

126. The method of embodiment 123, wherein prior to step a), the cell culture harvest is contacted with MgCl₂ and an endonuclease.

127. The method of embodiment 126, wherein the cell culture harvest is from a cell culture volume of about 1,000 L, about 1,500 L, about 2,000 L, or about 2,500 L.

128. The method of embodiment 124 or 126, wherein the endonuclease is Benzonase® endonuclease.

129. The method of embodiment 126, wherein the cell culture harvest is cooled to 2-8° C. before being contacted with MgCl₂ and the endonuclease.

130. The method of any one of embodiments 122 to 129, further comprising a step of virus inactivation after the multimodal anion exchange chromatography (MAC) step and before the hydrophobic interaction chromatography (HIC) step.

131. The method of embodiment 130, wherein the virus inactivation step comprises adjusting the pH of the solution obtained from step b) to about 3.4-3.6.

132. The method of embodiment 131, wherein after incubating the solution tor at least one hour, the pH is adjusted to about 7.0 before the hydrophobic interaction chromatography (HIC) step.

133. The method of any one of embodiments 130-132, further comprising a depth filtration step prior to the hydrophobic interaction chromatography (HIC) step.

134. The method of embodiment 133, wherein the depth filtration step follows the virus inactivation step.

135. The method of any one of embodiments 122 to 134, comprising a virus removal step after the hydrophobic interaction chromatography (HIC) step.

136. The method of embodiment 135, wherein the virus removal step comprises nanofiltration.

137. The method of embodiment 135 or 136, further comprising an ultrafiltration step after the virus removal step.

138. The method of any one of embodiments 135-137, comprising a second virus inactivation step after the virus removal step.

139. The method of embodiment 138, wherein the second virus inactivation step comprises adding a dimethylurea solution.

140. The method of embodiment 138 or 139, comprising an ultrafiltration step after the second virus inactivation step.

141. The method of embodiment 122 or 123, further comprising one or more ultrafiltration and/or nanofiltration steps.

142. The method of embodiment 122 or 123, further comprising one or more virus inactivation steps.

143. The method of embodiment 122 or 123, further comprising one or more virus removal steps.

144. The method of any one of embodiments 122 to 143, wherein of the recombinant lubricin glycoprotein comprises the amino acid sequence of amino acid residues 25-1404 of SEQ ID NO:1 or 2.

145. The method of any one of embodiments 122 to 144, wherein at least 30% of the molecular weight of the recombinant lubricin glycoprotein is from glycosidic residues.

146. The method of any one of embodiments 122 to 145, wherein at least 90% of O-glycosylation of the lubricin glycoprotein is core 1 glycosylation.

147. The method of any one of embodiments 122 to 146, wherein the lubricin glycoprotein comprises O-glycan species, wherein the O-glycan species comprise about 7% or more Gal-GalNAc, about 80% or more 2,3-NeuAc Core 1, about 3% or more 2*NeuAc Core 1, and about 1% or more 2,3-NeuGc Core 1.

148. The method of any one of embodiments 122 to 147, wherein the lubricin glycoprotein comprises about 50 μg or more NANA per mg of the lubricin glycoprotein.

149. The method of any one of embodiments 122 to 148, wherein the lubricin glycoprotein comprises about 10 μg or less NGNA per mg of the lubricin glycoprotein.

150. The method of any one of embodiments 122 to 149, wherein the lubricin glycoprotein comprises about 100 μg or more Gal per mg of the lubricin glycoprotein.

151. The method of any one of embodiments 122 to 150, wherein the lubricin glycoprotein comprises about 100 μg or more GalNAc per mg of the lubricin glycoprotein.

152. A recombinant lubricin glycoprotein obtained by the method according to any one of embodiments 122 to 151.

153 A pharmaceutical composition comprising the recombinant lubricin glycoprotein according to embodiment 152, and a pharmaceutically acceptable excipient.

154. The pharmaceutical composition of embodiment 153, wherein purity of the pharmaceutical composition is 95%, 96%, 97%, 98%, 99%, or greater, as determined by reversed phase chromatography (RPC).

155. The pharmaceutical composition of embodiment 153 or 154, comprising less than 1% of aggregates of the recombinant lubricin glycoprotein.

156. The pharmaceutical composition of any one of embodiments 153-155, comprising less than 1% of fragments of the recombinant lubricin glycoprotein.

157. The pharmaceutical composition of any one of embodiments 153-156, comprising ≤1,000 ng host cell protein/mg of recombinant lubricin glycoprotein (ng/mg), ≤900 ng/mg, ≤800 ng/mg, ≤700 ng/mg, ≤600 ng/mg, ≤500 ng/mg, ≤400 ng/mg, ≤300 ng/mg, ≤250 ng/mg, ≤200 ng/mg, ≤150 ng/mg, or ≤100 ng/mg.

158. The pharmaceutical composition of any one of embodiments 153-157, comprising ≤10,000 pg host cell DNA/mg of recombinant lubricin glycoprotein (pg/mg), ≤5,000 pg/mg, ≤1,000 pg/mg, ≤500 pg/mg, ≤100 pg/mg, ≤50 pg/mg, ≤10 pg/mg, or ≤5 pg/mg.

159. The pharmaceutical composition of any one of embodiments 153-158, comprising less than 8 endotoxin units (EU)/mL, less than 1 EU/mL, less than 0.1 EU/mL, or less than 0.01 EU/mL, as determined by a bacterial endotoxin test (BET).

160. The pharmaceutical composition of any one of embodiments 153-159, having a total aerobic microbial count (TAMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

161. The pharmaceutical composition of any one of embodiments 153-160, having a total combined yeast/mold count (TYMC) of less than 1 colony forming unit (CFU)/mL, less than 1 CFU/2 ml, less than 1 CFU/3 ml, less than 1 CFU/4 ml, less than 1 CFU/5 ml, less than 1 CFU/6 ml, less than 1 CFU/7 ml, less than 1 CFU/8 ml, less than 1 CFU/9 ml, or less than 1 CFU/10 ml.

162. The pharmaceutical composition of any one of embodiments 153-161, wherein the composition is stable at 5° C. for at least 24 months.

163. The pharmaceutical composition of any one of embodiments 153-162, wherein the composition is stable at 25° C. for at least 1 month.

164. The pharmaceutical composition of any one of embodiments 153-163, wherein the composition has an initial concentration of about 0.15 mg recombinant lubricin glycoprotein/ml (mg/ml), about 0.20 mg/ml, about 0.25 mg/ml, about 0.30 mg/ml, about 0.35 mg/ml, about 0.40 mg/ml, about 0.45 mg/ml, about 0.50 mg/ml, about 0.55 mg/ml, about 0.60 mg/ml, about 0.70 mg/ml, about 0.8 mg/ml, about 0.9 mg/ml, about 1.0 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/mi., about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.5 mg/ml, about 4.0 mg/ml, from about 0.15 mg/ml to about 3.0 mg/ml, from about 0.45 mg/ml to about 3.0 mg/ml, from about 0.15 mg/ml to about 0.45 mg/ml, from about 1.5 mg/ml to about 3.0 mg/ml, from about 1.5 mg/ml to about 3.5 mg/ml, from about 2.0 mg/ml to about 3.0 mg/ml, from about 1.5 mg/ml to about 2.5 mg/ml, or from about 2.0 mg/ml to about 4.0 mg/ml.

165. A method for treating an ocular surface disorder, comprising a step of administering the pharmaceutical composition of any one of embodiments 153-164 to a patient.

166. The method of embodiment 165, wherein the ocular surface disorder is dry eye disease.

167. A pharmaceutical composition according to any one of embodiments 32-43, 93-119, or 153-164 for treating an ocular surface disorder.

168. A pharmaceutical composition according to any one of embodiments 32-43, 93-119, or 153-164 for use in treating an ocular surface disorder.

169. The use of a pharmaceutical composition according to any one of embodiments 32-43, 93-119, or 153-164 for the manufacture of a medicament for the treatment of ocular surface disorders.

References in the disclosure to “the invention” are intended to reflect embodiments of the several inventions disclosed in this specification, and should not be taken as necessarily limiting of the claimed subject matter, in as much as the claims set forth the invention(s) for which patent protection is sought.

The following Examples illustrate the invention described above, but are not, however, intended to limit the scope of the invention in any way. Other test models known as such to the person skilled in the pertinent art can also determine the beneficial effects of the claimed invention.

### EXAMPLES

### Example 1

### Purification Process of Recombinant Lubricin

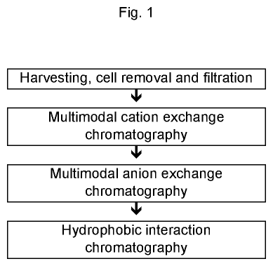

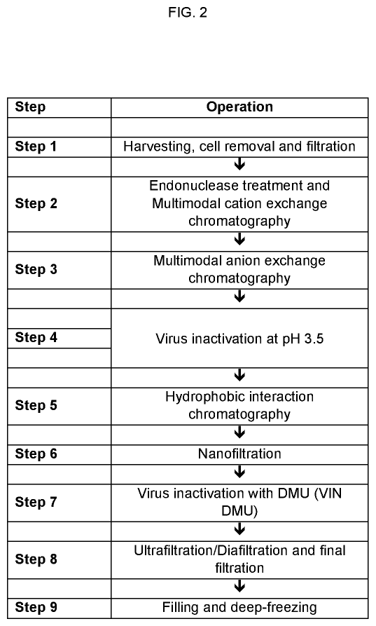

An exemplary purification process of lubricin is described in FIGS. 1 and 2 and Table 2 below. Generally, the process includes three chromatography steps and additional steps which are dedicated to virus inactivation (namely low pH incubation) and removal, nanofiltration, and, in an embodiment described in the following example, virus inactivation with N,N-Dimethylurea (DMU). At the end, the product is concentrated and diafiltrated into the final buffer.

TABLE 2
Process flow chart

| | | | Anticipated |
|---|---|---|---|
| | | | recovery |
| Step | Description | Parameters | (%) |
| 1 | Benzonase | Load pretreatment: 50 U_{Benzonase}/ml_{cell free harvest} | ~50% |
| | treatment and | Resin: Capto MMC | |
| | Multimodal | Equilibration: 20 mM Tris, pH 8.0 | |
| | Cation | Loading Conditions: 3-6 g/L_{packed resin} | |
| | Exchange | Wash 1: 20 mM Tris, pH 10 | |
| | Chromatography | Wash 2: Equilibration buffer | |
| | (MCC) (B/E | Elution: 20 mM Tris, 20 mM sodium acetate, 50 | |
| | mode) | mM L-arginine, 1M NaCl, pH 9 | |
| 2 | Multimodal | Resin: CaptoCore 700 | ~85% |
| | Anion Exchange | Equilibration: 50 mM sodium phosphate, 850 mM | |
| | Chromatography | NaCl, pH 7 | |
| | (MAC) (FT | Loading Conditions: pH 7.0, 10-18 g/L_{packed resin} | |
| | mode) | Wash: Equilibration buffer | |
| 3 | Virus | pH adjusted to 3.5. | ~92% |
| | Inactivation and | Incubation for 70 min at pH 3.5. pH adjusted to 7.0 | |
| | Neutralization | | |
| | (VIN) | | |
| 4 | Hydrophobic | Membrane: Sartobind Phenyl | ~98% |
| | interaction | Equilibration: 20 mM sodium phosphate, 1M | |
| | Chromatography | ammonium sulfate (AS), pH 7 | |
| | (HIC) (FT | Loading Conditions: 0.72M AS, 10-30 g/L_{packed resin} | |
| | mode) | Depth filter B1HC Pod, 100-200 L/m2 | |
| | | Wash: Equilibration buffer | |
| 5 | Nanofiltration | Pre-filter: Viresolve Pro Shield | ~95% |
| | (VRF) | Nanofilter: Planova 20N | |
| | | Nanofilter Load Ratio: ≤0.06 kg/m² | |
| 6 | Virus | Incubation with 3M DMU for 4 h | |
| | inactivation with | | |
| | DMU (VIN | | |
| | DMU) | | |
| 7 | Ultrafiltration, | Membrane: Pellicon 3, 30 kDa | ~90% |
| | microfiltration | Diafiltration Buffer: 10 mM sodium phosphate, 140 | |
| | (UFT) | mM NaCl, pH 7 | |
| 8 | Filling and | PET bottles | ~97% |
| | freezing (FIL) | | |
| | Overall Process Yield: | | ≥31% |

Starting material for purification was prepared from cell culture harvests containing recombinant human lubricin glyoprotein produced in a Chinese Hamster Ovary cell line (CHO-M cells as described in WO 2015/061488).

Step 1: Benzonase treatment and Multimodal Cation Exchange Chromatography (MCC)

Cells were removed with inline depth filtration or only by depth filtration, followed by 0.2 μm filtration. The cooled (2-8° C.) clarified cell-culture supernatant was spiked with Mg²⁺ and Benzonase® endonuclease (Merck MilliporeSigma, Burlington, Mass.) to a target concentration of 50′000 U/L_{clarified harvest} and incubated at 4° C. for 16 hours. Approximately 1.07 g of 1 M MgCl₂ solution (density 1.070 g/ml) was used per 1.0 kg of clarified harvest.

After Benzonase® treatment, the clarified harvest was applied to a Multimodal Cation exchange Chromatography (MCC) column packed to a bed height of 20 cm using Capto MMC resin (GE Healthcare Bio-Sciences, Pittsburgh, Pa.). Residence times larger or equal to 4 min were applied. Depending on the amount of product present, multiple cation exchange chromatography cycles were performed. Each cycle allows a maximum loading of approximately 6 g/L column volume.

Prior to loading, the column was primed with 100 mM Tris, pH 8 and then equilibrated with equilibration buffer (20 mM Tris, pH 8). After loading of the cell-free harvest, the column was washed first with a wash buffer (20 mM Tris, pH 10) and then with the equilibration buffer. The product was eluted with a buffer containing 20 mM Tris, 20 mM sodium acetate, 50 mM L-arginine, 1 M NaCl, pH 9.

### Step 2: Multimodal Anion Exchange Chromatography (MAC)

The filtered product-containing solution from the previous step was subjected to chromatographic polishing by multimodal anion-exchange chromatography (MAC) in flowthrough mode. The solution was applied to a Capto Core 700 column (GE Healthcare Life Sciences, Pittsburgh, Pa.) packed to a bed height of 20 cm. A residence time of larger or equal than 6 min was applied. Depending on the amount of product present, multiple multimodal anion exchange chromatography cycles were performed. Each cycle allowed a maximum loading of approximately 18 g/L column volume. The load was adjusted to pH 7.0 with 0.5 M phosphoric acid solution. The equilibration and the post-loading wash were performed with a buffer containing 50 mM sodium phosphate, 750 mM NaCl, pH 7. The product was collected in the percolates (flowthroughs).

### Step 3: Virus Inactivation (VIN)

The partially purified lubricin solution was then subjected to virus inactivation by adjusting the pH to pH 3.4-3.6 with 0.5 M phosphoric acid solution. After incubation at 17 -25° C. for 60-90 min, the pH was adjusted to pH 7.0 with 1 M tris(hydroxymethyl)aminomethane (Tris) solution. Finally, the solution was filtered through a 0.2 μm filter.

### Step 4: Hydrophobic Interaction Chromatography (HIC)

A second chromatographic polishing step was then performed using hydrophobic interaction chromatography (HIC) in flowthrough mode.

The filtered product-containing solution from the previous step was spiked with 3 M ammonium sulfate to a target concentration of 0.72 M ammonium sulfate concentration. After being filtered the spiked solution was applied to a Sartobind Phenyl membrane adsorber. A residence time of higher or equal to 0.2 min was applied. Depending on the amount of product present, multiple membrane adsorber cycles were performed. Each cycle allowed a maximum loading of approximately 30 g/L column volume.

The equilibration and the post-loading wash were performed with a buffer containing 20 mM sodium phosphate, 1 M ammonium sulfate, pH 7. The product was collected in the percolates (flowthroughs).

### Step 5: Nanofiltration (VRF)

After pre-filtration through a 0.1 μm filter, the lubricin-containing solution was subjected to nanofiltration using the Planova 20N virus reduction filter at an operating pressure differential of 0.8 bar. A maximum load of 60 g of product per m2 was applied. The feed pressure was kept constant and the flux decreased over time. A maximum flux decay of 80% was allowed in the manufacturing process.

Step 6: Virus Inactivation with 3 M DMU (VIN DMU)

The solution was then subjected to a virus inactivation step with 3 M N,N-Dimethylurea (DMU). The solution from the viral removal filtration step was mixed in a ratio of 1:1 (v/v) with 6 M DMU solution, and the mixture was incubated at room temperature for up to 360 minutes (no stirring). The solution was then filtered with a 0.45/0.2 μm Sartopore 2 microfilter (Sartorius AG, Germany).

### Step 7: Ultrafiltration and Compounding (UFT)

The solution was then subjected to ultrafiltration/diafiltration, which consisted of a concentration step and a diafiltration step with a buffer designed to achieve the drug substance target composition. The step used a 30 kDa cut-off membrane. Polysorbate 20 was added after the ultrafiltration/diafiltration process. The final DS solution was filtered through a 0.2 μm filter.

Table 3 shows the purification results of the process. In the table, HMW and LMW % were determined using SEC assays as described below. HCP content was measured by CHO-ELISA. DNA content was measured using qPCR.

TABLE 3
Process performance.

| | | | | | | | RPC | RPC | RPC |
|---|---|---|---|---|---|---|---|---|---|
| | | Yield | HMW | LMW | HCP | DNA | EP | Main Peak | LP |
| Step | | (%) | (%) | (%) | (ppm) | (ppb) | (%) | (%) | (%) |
| Clarified | | | 3.1 | 53.7 | 2,110,000 | 9,880,000 | | | |
| harvest* | | | | | | | | | |
| MCC | In | | — | — | — | — | | | |
| | Out | 45-75% | 0.8 | 26.2 | 91300 | 2920 | 32.4 | 46.4 | 21.2 |

| | | | | | | | | |
|---|---|---|---|---|---|---|---|---|
| MAC | 80-90% | — | — | — | — | — | — | — |
| VIN | 90-99% | 0.7 | 0.5 | 1130 | <307 | 32.5 | 67.3 | 0.3 |
| HIC | 93-100% | 2.0 | 0.3 | 131 | <476 | 32.6 | 67.4 | 0.0 |
| VRF | 95-99% | 2.1 | 0.4 | 146 | <515 | 33.7 | 66.3 | 0.0 |
| UFT | 92-95% | 0.6 | 0.2 | 284 | <119 | 34.1 | 65.9 | 0.0 |

HMW = high molecular weight; LMW = low molecular weight; HCP = host cell protein; RPC = Reversed Phase Chromatography; RPC EP = Reversed Phase Chromatography Early Peak; RPC LP = Reversed Phase Chromatography Late Peak.

Tables 4-1 to 4-7 show the typical process outputs for the various steps of the process.

TABLE 4-1
MCC - Output

| | | |
|---|---|---|
| | Lubricin Typical | |
| Parameter | Range | Comment |

| | |
|---|---|
| Yield | 45-75% |
| Pool Volume | 1.7-2.5 CV |
| Pool Concentration | 1.3-2.0 g/L |
| Aggregate Content (SE-HPLC) | 0.2-0.8% |
| Degradation Product Content | 27-45% |
| (SE-HPLC) | |
| Host Cell Protein Content | 410′000-557′000 ng/mg |
| DNA Content | Approx. 2920 pg/mg |
| Column Pressure Drop | ≤1.0 bar (0.6 bar |
| | increase during elution) |

TABLE 4-2
MAC - Output

| | | |
|---|---|---|
| | Lubricin Typical | |
| Parameter | Range | Comment |

| | |
|---|---|
| Yield | 80-90% |
| Volume Increase | 3-6% |
| Pool Concentration | 1.1-1.6 g/L |
| Aggregate Content (SE-HPLC) | 0.1-0.8% |
| Degradation Product Content | 4-9% |
| (SE-HPLC) | |
| Host Cell Protein Content | 1′000-7000 ng/mg |
| DNA Content | Approx. 400-600 |

TABLE 4-3
VIN - Output

| | | |
|---|---|---|
| | Lubricin Typical | |
| Parameter | Range | Comment |

| | |
|---|---|
| Yield | 90-99% |
| Overall Volume Increase | 11-12% |
| Concentration | 1.0-1.3 g/L |
| Conductivity | 60-70 mS/cm |
| Aggregate Content (SE-HPLC) | 0.2-0.9% |
| Degradation Product Content | 4-9% |
| (SE-HPLC) | |
| Host Cell Protein Content | 500-4000 ng/mg |
| DNA Content | Approx. 300-500 pg/mg |

TABLE 4-4
HIC - Output

| | Lubricin Typical | |
|---|---|---|
| Parameter | Range | Comment |
| Yield | 93-100% | |
| Volume Increase | 0-4% | Weight based |
| Pool Concentration | 0.6-0.8 g/L | |
| Aggregate Content (SE-HPLC) | 0.1-0.8% | |
| Degradation Product Content | 4-9% | |
| (SE-HPLC) | | |
| Host Cell Protein Content | 80-130 ng/mg | |
| DNA Content | Approx. 300-500 | |
| | pg/mg | |

TABLE 4-5
VRF - Output

| | | |
|---|---|---|
| | Lubricin Typical | |
| Parameter | Range | Comment |

| | |
|---|---|
| Yield | 95-99% |
| Pool Concentration | 0.5-0.7 g/L |
| Volume Increase | n.d. |
| Aggregate Content (SE-HPLC) | 0.1-0.8% |
| Degradation Product Content | 4-9% |
| (SE-HPLC) | |
| Initial Flux | 25-30 L/m²/h |
| Average Flux | 17-20 L/m²/h |
| Filtration Time | 4-5 h |

TABLE 4-6
VIN DMU - Output

| | | |
|---|---|---|
| | Lubricin Typical | |
| Parameter | Range | Comment |

| | |
|---|---|
| Yield | 90-99% |
| Overall Volume Increase | 100% |
| Concentration | 0.1-0.4 g/L |
| Conductivity | ≤70 mS/cm |

TABLE 4-7
UFT-Output

| | | |
|---|---|---|
| | Lubricin Typical | |
| Parameter | Range | Comment |

| | |
|---|---|
| Yield | 92-95% |
| Concentration 1 | |
| Feed Flow Rate | 4.6-5.6 L/min |
| Permeate Flux | 44-68 L/m²/h |
| Diafiltration | |
| Feed Flow Rate | 4.6-5 L/min |
| Permeate Flux | 45-55 L/m²/h |

TABLE 4-8
Final Drug Substance (DS) quality

| | | Lubricin | |
|---|---|---|---|
| Parameter | Target | Typical Values | Comment |
| Product Concentration | 2.0 ± 0.5 g/L | 2.0 | |
| DS pH | 7.0 ± 0.5 | 7.0 | |
| Phosphate Content | Approximately | 10.7-10.9 mM | |
| | 10 mM | | |
| NaCl Content | Approximately | n.a. | |
| | 140 mM | | |
| Polysorbate 20 Content | 0.02% (w/v) | n.a. | |
| Aggregate Content | ≤15% | 0.1-0.8% | |
| (SE-HPLC) | | | |
| Fragment Content | ≤15% | 2.5-9.0% | |
| (SE-HPLC) | | | |
| Host Cell Protein Content | ≤1000 ng/mg | 100-300 ng/mg | |
| Residual DNA | ≤10′000 pg/mg | 100-300 pg/mg | For a dose of |
| Bacterial Endotoxin Test | <8 EU/mL | | 50 uL this is |
| (BET) | | | the equivalent |
| | | | of ≤0.03 ng/dose |
| | | | Depends on |
| | | | maximum dose and |
| | | | mode of admission. |

The final lubricin drug substance solution contains approximately 10 mM sodium phosphate, 140 mM sodium chloride, and 0.02% (w/v) polysorbate 20 in addition to the lubricin protein.

### Example 2

### Purification Process of Recombinant Lubricin

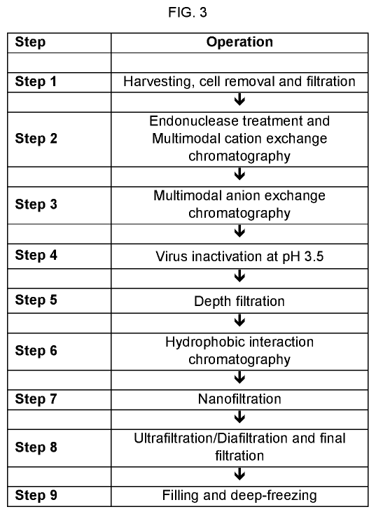

A further purification process of lubricin is described in FIG. 3. Generally, the process is similar to the process described in Example 1, but does not include a step of virus inactivation with N,N-Dimethylurea (DMU).

Starting material for purification was prepared from cell culture harvests containing recombinant human lubricin glycoprotein produced in a Chinese Hamster Ovary cell line (CHO-M cells as described in WO 2015/061488). Cells were first cultured in a Wave bioreactor in a cell culture volume of approximately 20 L, and then further cultured in a WAVE bioreactor in 2 pre-stages with increasing cell culture volume of approximately 100 L to 400 L. Finally, cells were cultured in a 2,000 L bioreactor.

The residual host cell protein at various stages of the purification process are shown in Table 5-1 below:

TABLE 5-1
Residual host cell protein in ng/mg glycosylated protein

| | Host cell protein |
|---|---|
| Step | concentration (ng/mg) |
| Cell-free harvest | 1,860,000; 2,000,000 |
| MCC eluate pool | 267,000 |
| MAC percolate pool (flow-through) | 3,230 |
| VIN filtrate | 2,160 |
| HIC percolate pool (flow-through) | 237 |
| VRF filtrate | 202 |

The residual host cell DNA at various stages of the purification process are shown in Table 5-2 below:

TABLE 5-2
Residual host cell DNA in pg/mg glycosylated protein

| | Host cell DNA |
|---|---|
| Step | concentration (pg/mg) |
| Cell-free harvest | 45,400; 34,500 |
| MCC eluate pool | 603 |
| MAC percolate pool (flow-through) | <11.4 |
| VIN filtrate | <7.2 |
| HIC percolate pool (flow-through) | <58.8 |
| VRF filtrate | <60.2 |

Residual benzonase at various stages of the purification process are shown in Table 5-3 below:

TABLE 5-3
Residual benzonase in ng/mg glycosylated protein

| Step | Benzonase concentration (pg/mg) |
|---|---|
| MCC eluate pool | 15.3 |
| VIN filtrate | <2.5 |
| HIC percolate pool (flow-through) | <10.0 |
| VRF filtrate | <10.0 |
| Drug substance | <2.5 |

Table 5-4 shows various test requirements for drug substance batches made using the process, and the test results.

TABLE 5-4
Drug substance batch tests, release requirements, and values

| | | Value/ |
|---|---|---|
| Test | Release Requirement | Comment |
| Appearance of solution | Not more than 30 NTU | 2 NTU |
| | (equally or less opalescent | |
| | than Ph. Eur. reference | |
| | suspension IV) | |
| Color | Colorless to slightly | B9 |
| | brownish-yellow, not more | |
| | intensely colored than | |
| | reference solution BY4 (Ph. | |
| | Eur.) | |
| pH value | 6.5-7.5 | 6.9 |
| Identity by SEC | Difference in sample and | complies |
| | reference elution time not | |
| | to exceed ±5.0% | |
| A375 cell adhesion assay | Sample must show dose | complies |
| | dependent response | |
| | 50-150% relative biological | 105% |
| | activity compared to | |
| | reference substance | |
| Purity by RPC | Purity (%) | 99.0% |
| Aggregates by SEC | Sum of aggregates ≤15% | <1.0% |
| Purity/Fragments by SEC | Purity (monomer) ≥70% | 99% |
| | Sum of fragments ≤15% | <1.0% |
| Determinatoin of CHO host | ≤1000 ng/mg Active | 233 ng/mg |
| cell protein by ELISA | ingredient | |
| CHO residual DNA | ≤10000 pg/mg Active | <4 pg/mg |
| determination by | ingredient | |
| Quantitative PCR | | |
| Sialic acids | NANA (μg/mg drug | 173 μg/mg DS |
| | substance (DS)) | |
| | NGNA (μg/mg DS) | 0.4 μg/mg DS |
| Monosaccharides | Galactose (μg/mg DS) | 234 μg/mg DS |
| | GalNAc (μg/mg DS) | 286 μg/mg DS |
| Assay by SEC | 1.50-3.00 mg/ml | 2.05 mg/ml |
| Bacterial Endotoxins Test | <8 EU/ml | <1 EU/ml |
| (BET) | | |
| Microbial Enumeration Test | Total aerobic microbial | <1 CFU/10 ml |
| (MET) | count (TAMC) ≤10 | |
| | CFU/10 ml | |
| | Total combined yeast/ | <1 CFU/10 ml |
| | molds count (TYMC) ≤10 | |
| | CFU/10 ml | |

### Purity/Fragments by Size Exclusion Chromatography (SEC)

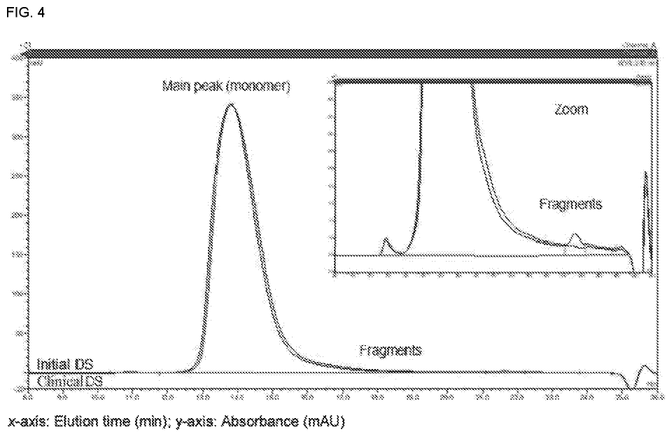

Recombinant lubricin degradation products (fragmentation products) included in the final composition were analyzed by a dedicated Size Exclusion Chromatography (SEC) method. Sample separation was performed based on size, and UV absorbance at 210 nm was recorded. Overlay chromatograms of an initial drug substance (DS) batch and a clinical drug substance batch are shown in FIG. 4. The peak is of approximately 0.4 peak area percentage (below LOQ) and was detected in the initial batch only. The sum of fragments for both batches was below LOQ (<1.0%). SEC profiles were overall similar for the two batches indicating that both batches were comparable by SEC analysis for purity and fragments.

### Aggregates by Size Exclusion Chromatography (SEC)

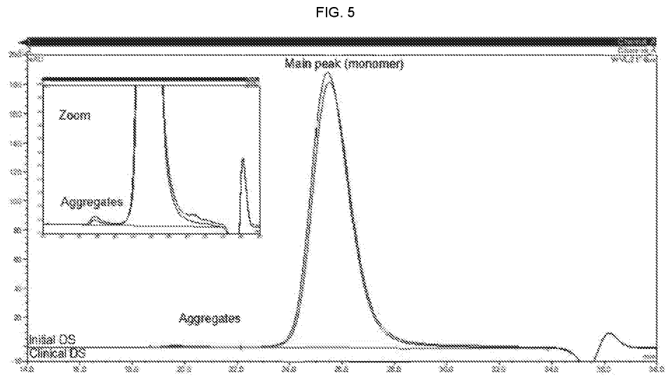

Degradation products with regard to aggregates were analyzed by a Size Exclusion Chromatography (SEC) method. Sample separation was performed based on size, and UV absorbance at 210 nm was recorded. Overlay chromatograms of an initial drug substance batch and a clinical drug substance batch profiles are shown in FIG. 5. The sum of aggregates for each batch was below the limit of quantification (LOQ; <1.0%). SEC profiles were similar for the two batches, indicating that the initial DS batch and the clinical DS batch were comparable by SEC for aggregates.

### Purity by Reversed Phase Chromatography (RPC)

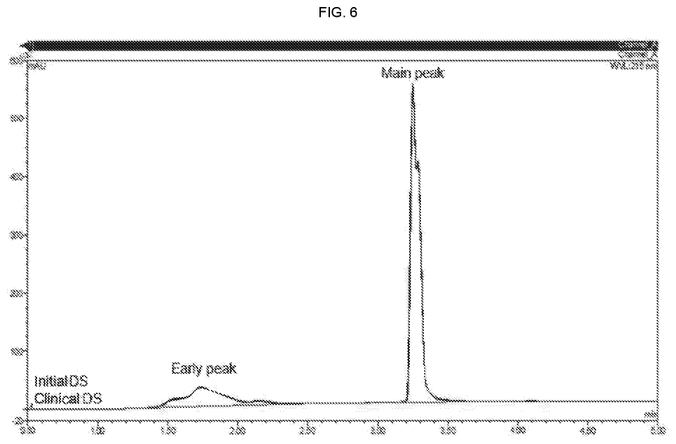

Purity by Reversed-Phase Chromatography (RPC) based on hydrophobicity was assessed, and UV absorbance at 215 nm was recorded. Overlay chromatograms of an initial drug substance batch and a clinical drug substance (DS) batch are shown in FIG. 6. Profiles and purities were similar for the two batches, indicating that the initial DS batch and the clinical DS batch were comparable by RPC. The early eluting peak was heterogeneous and integrated as one group of peaks. The identity of the early peaks was demonstrated by peptide mapping to include N- and C-termini truncated lubricin. Peak area percentage of the early eluting peak was similar for the two batches (approximately 23%). The main peak was also heterogeneous, and included two major and closely associated peaks. The main peak likely includes non-fragmented lubricin. The appearance of two peaks may indicate different molecular or structural conformations of the purified protein, which were observed by analytical ultracentrifugation.

### Molecular Mass Determination by Analytical Ultracentrifugation

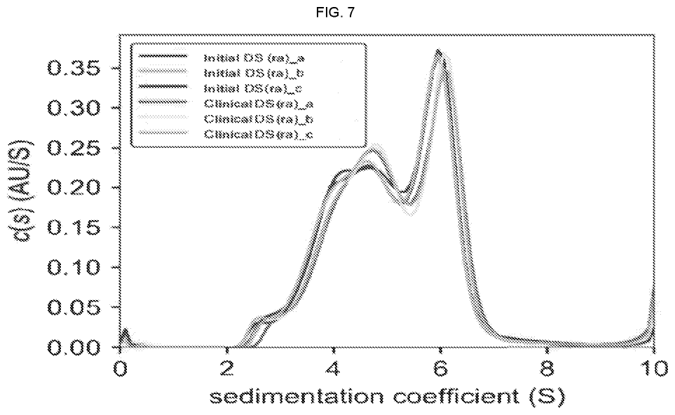

The initial drug substances (DS) batch and clinical DS batch were measured under native conditions by analytical ultracentrifugation (AUC) using sedimentation equilibrium (SE-AUC) and sedimentation velocity (SV-AUC) modes, respectively. SE-AUC measures the molecular weight while SV-AUC measures molecular properties, for example, as conformation and size-distribution. Samples were introduced in 12 mm 6-channel centerpieces (SE-AUC) and 12 mm 2-channel centerpieces (SV-AUC) and analyzed according to set conditions at a temperature of 20° C. FIG. 7 shows the SV-AUC absorbance profiles at 230 nm of each DS batch, measured in triplicate. Both DS batches demonstrated a similar profile that includes two major peaks with maxima around approximately 4.5 S and 6 S. The two bands likely correspond to different structural conformations of similar molecular weight: a more elongated form (4.5 S) and a relatively more compact form (6 S). Peak areas were similar for each DS batch.

The molecular weight as measured by SE-AUC of the initial DS batch was 294,938.7 g/mol. The molecular weight as measured by SE-AUC of the clinical DS batch was 291,931,9 g/mol. Molecular weights of the two batches were similar and within the expected range. The minor difference in observed molecular weights between the two batches was within the range of error of the method.

The theoretical molecular mass of recombinant human lubricin based on the amino acid composition is 148,308 Da. The additional measured mass of around 145 kDa per DS batch likely consists primarily of sialylated O-glycans. No aggregate species were detected in the batches. Thus, the initial DS batch and clinical DS batch showed similar AUC profiles and molecular weights.

### Example 3

The following assays were used and can be used for analyzing solutions purified using the methods provided herein.

### Analytical Assay: Identity and Aggregates by Size Exclusion Chromatography (SEC)

Aggregates of lubricin in a sample from the process described above are separated from monomer based on size under native conditions using Size Exclusion Chromatography (SEC) with UV detection. The amount/content of aggregate is determined as a percentage of the total area obtained for each sample determined. Identity of the sample is assessed relative to a reference standard of known identity. Identity or Aggregate determination can be performed stand-alone or combined.

This method is applicable for drug substance and drug product generally referred to as ‘sample’.

The following test solutions are used:

| | |
|---|---|
| Mobile Phase | 50 mM sodium phosphate/400 mM sodium perchlorate, |
| | pH 7.0 |
| Diluent (sample diluent) | 10 mM sodium phosphate/140 mM sodium chloride/ |
| | 0.02% (w/v) polysorbate 20 (PS20), pH 7.0 |
| BSA/Thyroglobulin/NaCl | e.g. dissolve 100 ± 10 mg BSA, 100 ± 10 mg |
| (saturation solution) | Thyroglobulin and 100 ± 10 mg NaCl in ca. 80 mL water. |
| | Fill to a final volume of 100 mL with water. Filter through |
| | a 0.45 μm (or less) membrane filter. |
| Molecular Weight Marker | I. Prepare 150 mM potassium phosphate, pH 6.5 |
| Solution | II. Dissolve 50 ± 1 mg of thyroglobulin (669 kDa), 20 ± 1 |
| (MWM solution) | mg of IgG (150 kDa), 25 ± 1 mg of holo-transferrin (80 |
| | kDa), 25 ± 1 mg of ovalbumin (45 kDa), 20 ± 1 mg of |
| | carbonic anhydrase (29 kDa), 20 ± 1 mg of Aprotinin (6.5 |
| | kDa) and 16 ± 1 mg of histidine (209.6 Da) in approx. 70 |
| | mL 150 mM potassium phosphate, pH 6.5. Stir the |
| | solution gently for approx. 15 min. Fill up to a final |
| | volume of 100 mL with 150 mM potassium phosphate, pH |
| | 6.5, in a volumetric flask and filter through a 0.22 μm |
| | membrane filter. |

A lubricin sample solution is diluted to approximately 0.15 mg/mL (e.g. dilute 15 μL of the sample, reference at around 1 mg/mL with 85 μIL diluent). The sample diluent is 10 mM sodium phosphate/140 mM sodium chloride/0.02% (w/v) polysorbate 20 (PS20), pH 7.0. The lubricin reference solution is diluted in two steps to obtain a final lubricin concentration (LOQ solution) of 1.5 μg/mL. First, 30 μL of the reference solution is diluted at 0.15 mg/mL with 970 μL sample diluent. This was named solution A (approx. 4.5 μg/mL lubricin). Second, 100 μL of solution A was diluted with 200 μL sample diluent. This was named LOQ solution (1.5 μg/mL).

A Molecular Weight Marker Solution (MWM solution) is prepared as follows: 1) prepare 150 mM potassium phosphate, pH 6.5; and 2) Dissolve 50±1 mg of thyroglobulin (669 kDa), 20±1 mg of IgG (150 kDa), 25±1 mg of holo-transferrin (80 kDa), 25±1 mg of ovalbumin (45 kDa), 20±1 mg of carbonic anhydrase (29 kDa), 20±1 mg of Aprotinin (6.5 kDa) and 16±1 mg of histidine (209.6 Da) in approx. 70 mL 150 mM potassium phosphate, pH 6.5. The solution is stirred gently for approximately 15 minutes, and then filled up to a final volume of 100 mL with 150 mM potassium phosphate, pH 6.5, in a volumetric flask and filtered through a 0.22 μm membrane filter.

The following chromatographic conditions are used:

| | |
|---|---|
| Flow rate | 0.3 mL/min |
| Column temperature | 30° C. |
| | Chromeleon ™ settings: delta: 3° C. |
| Autosampler temperature | 5° C. |
| | Chromeleon ™ settings: Lower limit 4° C./ |
| | Upper limit 8° C. |
| Detection | 210 nm |
| | Chromeleon ™settings: Step: 1.0 s/Average: On |
| Data sampling rate | Not applicable |
| Injection volume | 20 μL (MWM solution 2 μL) |
| Run Time | 50 min |

Sequences are run as follows:

| | No. of | Inj vol |
|---|---|---|
| Sample name | injections | (μL) |
| MWM solution | 1 | 2 |
| Blank (diluent) ¹⁾ | 1 | 20 |
| Blank (diluent) ²⁾ | 1 | 20 |
| LOQ solution | 1 | 20 |
| Reference solution | 1 | 20 |
| Sample 1 solution | 1 | 20 |
| Sample 2 solution | 1 | 20 |
| . . . | . . . | 20 |
| Sample N solution (N ≤ 15) | 1 | 20 |
| Reference solution | 1 | 20 |
| MWM solution | 1 | 2 |

¹⁾ This blank is to exclude Aprotinin carry-over from the MWM solution into the next run
²⁾ This blank is used for noise calculation for LOQ determination and to assess interference

The above approach can be used for up to 15 samples. For more than 15 samples, the following sequence of injections can be used:

| | No. of | Inj vol |
|---|---|---|
| Sample Name | injections | (μL) |
| MWM solution | 1 | 2 |
| Blank (diluent) ¹⁾ | 1 | 20 |
| Blank (diluent) ²⁾ | 1 | 20 |
| LOQ solution | 1 | 20 |
| Reference solution | 1 | 20 |
| Sample 1 solution | 1 | 20 |
| Sample 2 solution | 1 | 20 |
| . . . | . . . | 20 |
| Sample 15 solution | 1 | 20 |
| Reference solution | 1 | 20 |
| Sample 16 solution | 1 | 20 |
| . . . | . . . | 20 |
| Sample N solution | 1 | 20 |
| Reference solution | 1 | 20 |
| MWM solution | 1 | 2 |

¹⁾ This blank is to exclude Aprotinin carry-over from the MWM solution into the next run
²⁾ This blank is used for noise calculation for LOQ determination and to assess interference

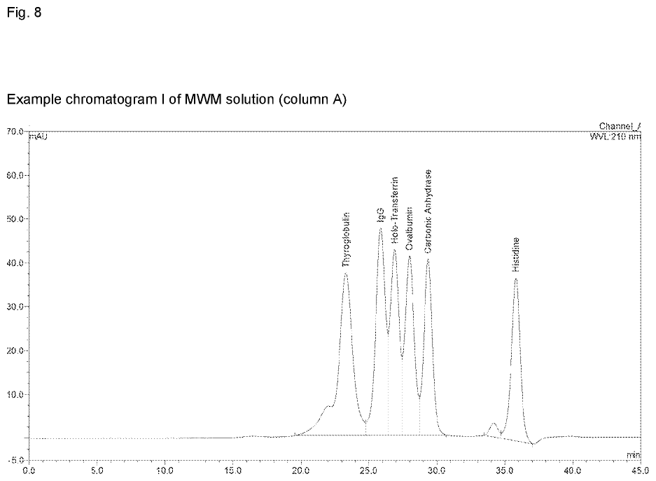

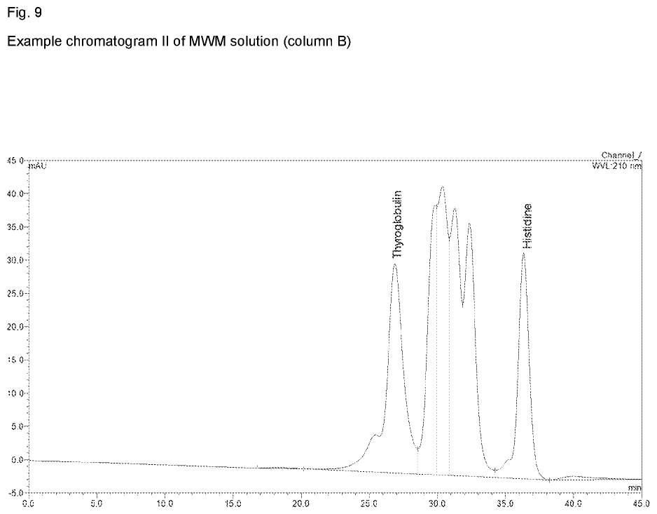

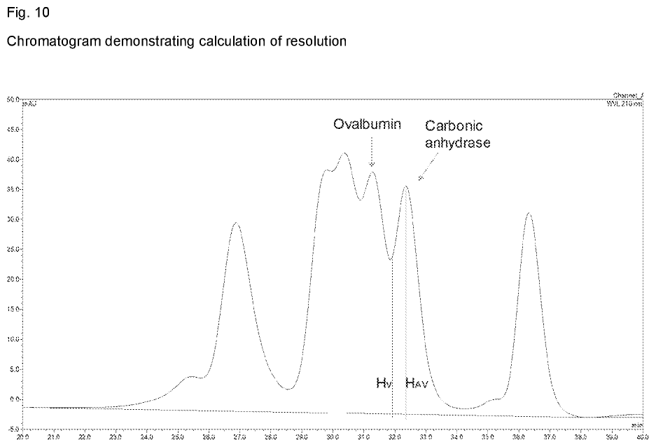

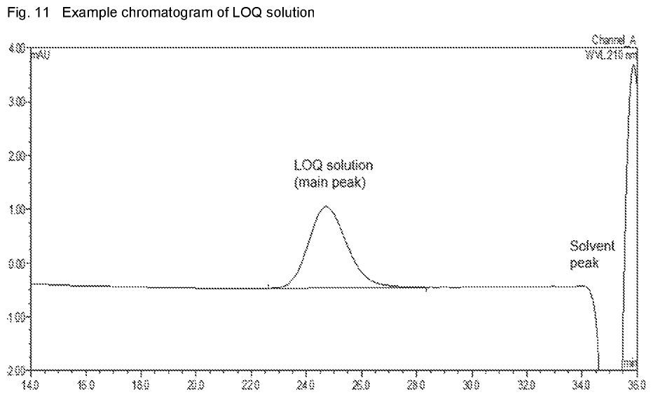

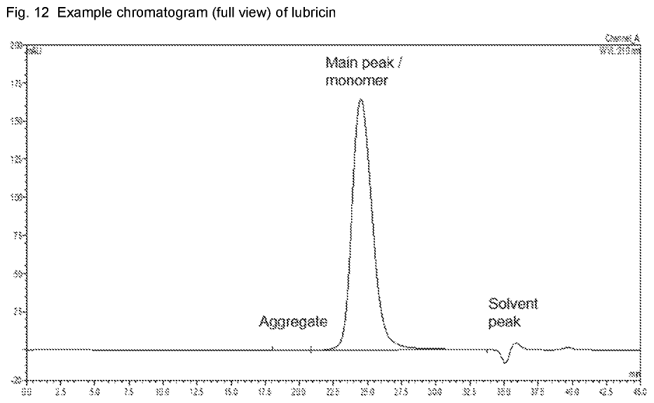

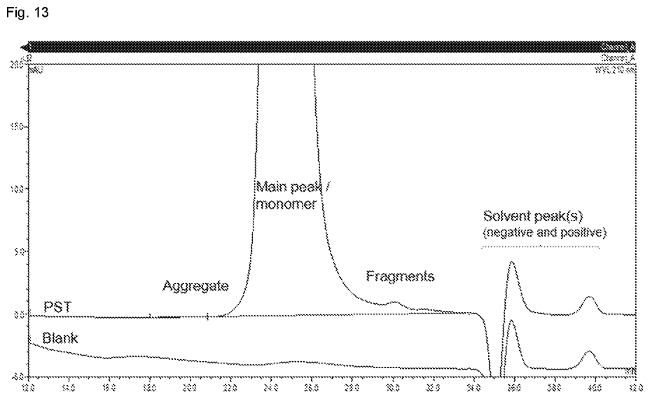

If more than 30 samples are to be analysed proceed with repeated injections of Reference solution accordingly after sample N (30, 45, etc.). The results for the sample injections are valid as long as the SST criteria shown below are met before and after the sample injections (bracketing approach).

| | |
|---|---|
| Visual | The peak pattern of the MWM proteins |
| assessment | corresponds to the comparison chromatograms in |
| of MWM | FIG. 8 or FIG. 9 or similar profiles (columns may |
| solution | demonstrate different selectivity and thus different |
| | peak profiles) |
| | Due to variations in quality of the individual |
| | molecular weight marker proteins, it is possible that |
| | additional small peaks may be observed. |
| Resolution | The peak resolution R is calculated for the first and |
| | last MWM solution injections to assess the column |
| | performance, see FIG. 10 |
| | Requirement: R must be ≥ 1.4 |
| | $R = \frac{{H}_{AV}}{{h}_{V}}$ |
| | H_{AV}: Peak height of Carbonic anhydrase |
| | h_{V}: Height of valley between Ovalbumin and |
| | Carbonic anhydrase |
| Interference | No interfering peak detected in blank runs (blank |
| | runs after the LOQ solution only as defined in the |
| | sequence of injections) with a signal height ≥ LOQ |
| | signal height, in the integrated range of the |
| | chromatogram of the sample |
| LOQ | Signal-to-noise ratio (S/N) for the lubricin peak in |
| | the LOQ solution must be ≥ 10. See FIG. 11 |
| | Calculation: |
| | $\frac{S}{N} = \frac{2 H}{h}$ |
| | H: Height of the lubricin peak |
| | h: Height of the background noise in the |
| | chromatogram observed over a distance equal to five |
| | times the width at half-height of the peak in front and |
| | after the peak in the chromatogram or in the blank run. |
| | Example for Chromeleon™ software settings |
| | For Chromeleon™ chromatography data system |
| | version 6.8, the blank injection before LOQ solution |
| | can be used for the calculation of the noise over a |
| | range of 5 times the peak width at half peak height, |
| | within the time window of the lubricin protein peak in |
| | the LOQ solution injection. |
| | In the SST-Properties window set “Parameter Input |
| | for ‘Signal to Noise Ratio’” as 5 times of the “Peak |
| | Width at 50% Height”. |
| Visual | Peak profiles of all reference solution injections |
| assessment | must be visually comparable within the sequence. |
| of reference | Peak profiles of all reference solution injections |
| | should be comparable to the example chromatograms |
| | in FIG. 12 and FIG. 13. The requirement is linked to |
| | the reference in use. |
| Consistency | The total peak area (aggregate, main variant and |
| of dilution | fragments) of the sample injections must not differ |
| (concentration | more than 20% from the first reference injection. |
| range) | |
| | $0.80 \leq \frac{{a}_{sample}}{{a}_{ref}} \leq 1.2$ |
| | aₛₐₘₚₗₑ: total peak area for each individual sample |
| | injection (mAU * min) |
| | a_{ref}: total peak area for the first reference |
| | injection (mAU * min) |

If more than 30 samples are to be analyzed proceed with repeated injections of Reference solution accordingly after sample N (30, 45, etc.). The results for the sample injections are valid as long as the SST criteria are met before and after the sample injections (bracketing approach).

The results are evaluated as follows:

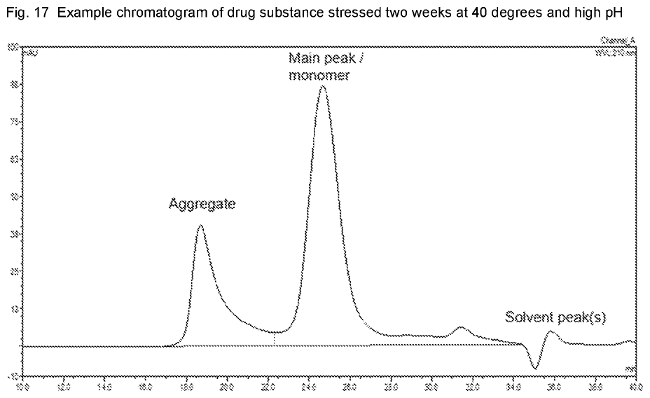

As a general rule, draw one baseline from aggregate peak(s) to solvent peak, see example chromatograms FIG. 12 and FIG. 13 and stressed sample FIG. 17.

### Peak Integration and Numbering

If more than one aggregate exists, separate the peaks from each other and from main peak (monomer), by an orthogonal split at the minimum of the valley separating them. If the main peak and aggregate peak are resolved by a shoulder only a split may be set at the inflection point of the shoulder. No split should be set between main peak and fragments (integrated as one unit).

Do not integrate the solvent peak and peaks from the blank if existent, for integration of reference, LOQ and sample solutions.

Aggregate peaks are identified according to their elution time relative to the main peak. Peaks eluting before the main are assigned to aggregation products (e.g. named APx).

### Aggregate Calculation

Determine the peak area (mAU*min) of all integrated aggregate peaks and main peak+fragments (main peak and fragments are integrated as one unit).

Calculate for each sample the area percentage (% P) of each aggregate peak. Calculate the sum (%) of relative peak areas of aggregates. Peaks<1.0% (LOQ) are not included in the calculation of the sum aggregates.

The % P of aggregate peak is calculated according to the following formula:

$$\% P = \frac{{a}_{rs}}{{a}_{t}} \times 100$$

aᵣₛ: Peak area of a product-specific aggregate peak in the sample solution chromatogram (mAU*min)
aₜ: Total peak area of the sample solution chromatogram (mAU*min), i.e. the sum area of all integrated peaks (aggregates+monomer/fragments).

### Elution Time Difference

Elution time difference defined as A (delta) time difference of main peak and aggregate peak(s) (above LOQ), i.e. Time main peak (min)−Time aggregate peak (min).

### Identity

Identity of sample is assessed by comparing the elution time of main peak of sample relative to the average of the elution times (first and last in the sequence only) of reference standard of known identity. Identity testing can be performed stand-alone or combined with aggregate determination.

Identity of placebo is assessed by absence of appearance of sample peak above LOQ at the expected elution time of reference.

Calculation: Difference=[(Ts−Tr)/Tr]*100
Ts=elution time of sample
Tr=elution time of reference (average)

### Reporting

Aggregate amount: report the peak area percentage (% P) of sum aggregate products and % P of each aggregate peak.

Report for information the A (delta) time difference for each aggregate peak versus main peak rounded to one decimal, X.X min.

Identity: report the difference in elution times (in percentage).

### Example 4

The following assays were used and can be used for analyzing solutions purified using the methods provided herein.

### Analytical Purity Assay: Fragments by Size Exclusion Chromatography (SEC)

Fragments of lubricin are separated from monomer based on size under native conditions using Size Exclusion Chromatography (SEC) with UV detection. Purity (monomer) and the amount of fragments are determined as a percentage of the total sample area obtained for each sample. Assay is determined based on total sample peak area versus total peak area of a reference of known concentration. Assay or Purity can be performed stand-alone if required.

This method is applicable for drug substance and drug product generally referred to as ‘sample’.

The following chromatographic conditions are used:

The following test solutions are used:

| | |
|---|---|
| Flow rate | 0.15 mL/min |
| Column temperature | 30° C. |
| | Chromeleon ™ settings: delta: 3° C. |
| Autosampler | 5° C. |
| temperature | Chromeleon ™ settings: Lower limit: 4° C./ |
| | Upper limit: 8° C. |
| Detection | 210 nm |
| | Chromeleon ™ settings: Step: 1.0 s/Average: On |
| Injection volume | 20 μL (MWM solution 2 μL) |
| Run Time | 35 min |

| | |
|---|---|
| Sample/ | For purity testing only, prepare a single preparation of |
| reference | reference and sample, respectively (e.g., one preparation |
| solution | from one ampoule for the reference; one preparation from |
| (0.15 mg/mL) | one vial per sample). |
| Single | For assay (and in combination with purity), prepare three |
| preparation | individual preparations of reference sample using one |
| | ampoule (e.g., from the sample ampoule is prepared three |
| | individual dilutions) and three individual preparations of |
| | sample (e.g., from the same sample vial is prepared three |
| | individual dilutions), respectively. |
| | Dilute the lubricin sample, reference with diluent to |
| | approx. 0.15 mg/mL. Pipette the diluent first, then the |
| | aliquot of sample, reference, and vortex briefly (about 3 s |
| | on a medium setting). |
| | e.g. dilute 15 μL of the sample, reference at around 1 |
| | mg/mL with 85 μL diluent |
| | Note: if sample concentration is around 0.15 mg/mL no |
| | dilution is necessary. Sample can be injected as such (tel |
| | quel, undiluted) |
| LOQ solution | Dilute in two steps the lubicin reference solution to |
| (1.0%, | obtain a final lubricin concentration (LOQ solution) |
| 1.5 μg/mL | of 1.5 μg/mL. |
| lubricin) | e.g. First, dilute 30 μL of the reference solution at 0.15 |
| Single | mg/mL with 970 μL sample diluent. This is named |
| preparation | solution A (approx. 4.5 μg/mL lubricin). |
| | e.g. Second, dilute 100 μL of solution A with 200 μL |
| | sample diluent. This is named LOQ solution (1.5 μg/mL) |
| Blank | Sample diluent |

Sequences are run as follows for Purity and Assay:

| | No. of | Inj vol |
|---|---|---|
| Sample name | injections | (μL) |
| Saturation solution (weekly | 5 | 20 |
| recurrence) ^{1), 2)} | | |
| Blank (diluent) ¹⁾ | 1 | 20 |
| MWM solution | 1 | 2 |
| Blank (diluent) ³⁾ | 1 | 20 |
| LOQ solution | 1 | 20 |
| Reference solution | 1/3 ⁴⁾ | 20 |
| Sample 1 solution | 1/3 ⁴⁾ | 20 |
| Sample 2 solution | 1/3 ⁴⁾ | 20 |
| . . . | . . . | 20 |
| Sample N solution (N ≤ 15) | 1/3 ⁴⁾ | 20 |
| Reference solution | 1/3 ⁴⁾ | 20 |
| MWM solution | 1 | 2 |

¹⁾ The five time injection of Saturation solution followed by a Blank is performed once per week only for a column-in-use (assuming the column is consecutively used on a weekly basis). The same procedure applies after column storage of an already used column. Alternatively, to condition a column in use, calculate the tailing factor TF for the last 5 injections of the reference solution. The TF must be below the SST limit.
²⁾ Alternatively, run a first set of Reference solution tel quel (2 injections, 20 μL injection volume) and a second set of Reference solution (0.15 mg/ml, 10 injections, 20 μL injection volume).
³⁾ This blank is used for noise calculation for LOQ determination and to assess interference (according to SST, described herein).
⁴⁾ Single injection for Purity evaluation only (one vial). For Assay evaluation, stand-alone or combined with Purity, single injection from three different vials (individually prepared).

For more than 15 samples, the following sequence is used.

| | No. of | Inj vol |
|---|---|---|
| Sample Name | injections | (μL) |
| Saturation solution (weekly | 5 | 20 |
| recurrence) ^{1), 2)} | | |
| Blank (diluent) ¹⁾ | 1 | 20 |
| MWM solution | 1 | 2 |
| Blank (diluent) ³⁾ | 1 | 20 |
| LOQ solution | 1 | 20 |
| Reference solution | 1/3 ⁴⁾ | 20 |
| Sample 1 solution | 1/3 ⁴⁾ | 20 |
| Sample 2 solution | 1/3 ⁴⁾ | 20 |
| . . . | . . . | 20 |
| Sample 15 solution | 1/3 ⁴⁾ | 20 |
| Reference solution | 1/3 ⁴⁾ | 20 |
| Sample 16 solution | 1/3 ⁴⁾ | 20 |
| . . . | . . . | 20 |
| Sample N solution | 1/3 ⁴⁾ | 20 |
| Reference solution | 1/3 ⁴⁾ | 20 |
| MWM solution | 1 | 2 |

¹⁾ The five time injection of Saturation solution followed by a Blank is performed once per week only for a column-in-use (assuming the column is consecutively used on a weekly basis). The same procedure applies after column storage of an already used column. Alternatively, to condition a column in use, calculate the tailing factor TF for the last 5 injections of the reference solution. The TF must be below the SST limit.
²⁾ Alternatively, run a first set of Reference solution tel quel (2 injections, 20 μL injection volume) and a second set of Reference solution (0.15 mg/ml, 10 injections, 20 μL injection volume).
³⁾ This blank is used for noise calculation for LOQ determination and similarly to assess interference (according to SST, described herein).
⁴⁾ Single injection for Purity evaluation only (one vial). For Assay evaluation, stand-alone or combined with Purity, single injection from three different vials (individually prepared).

If more than 30 samples are to be analysed proceed with repeated injections of Blank and Reference solution accordingly after sample N (30, 45, etc.). The results for the sample injections are valid as long as the SST criteria are met before and after the sample injections (bracketing).

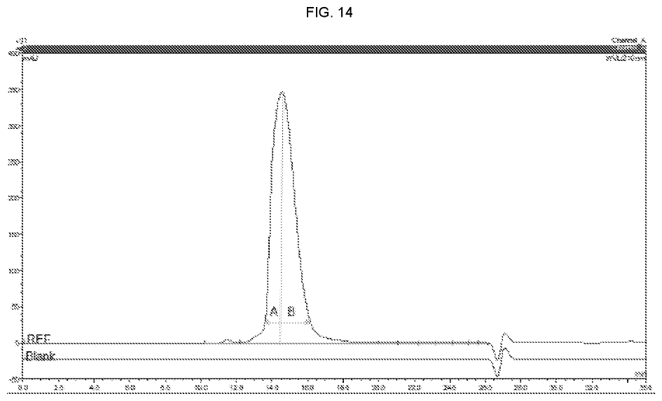

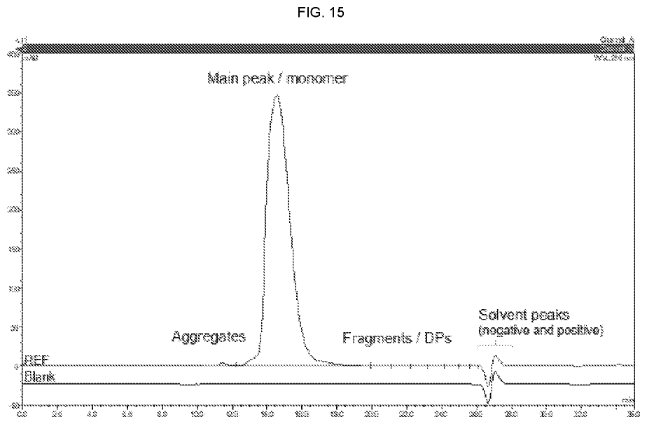

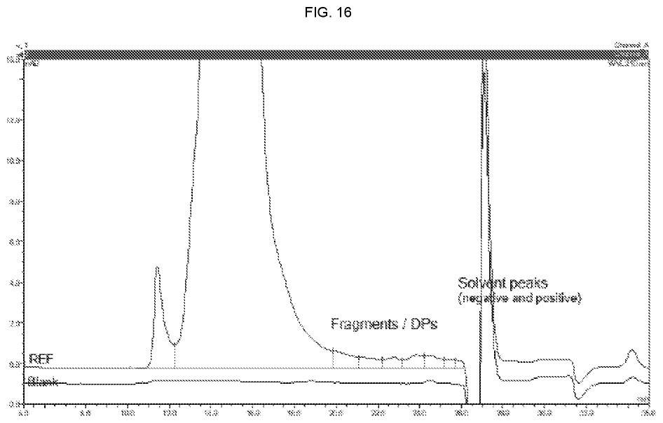

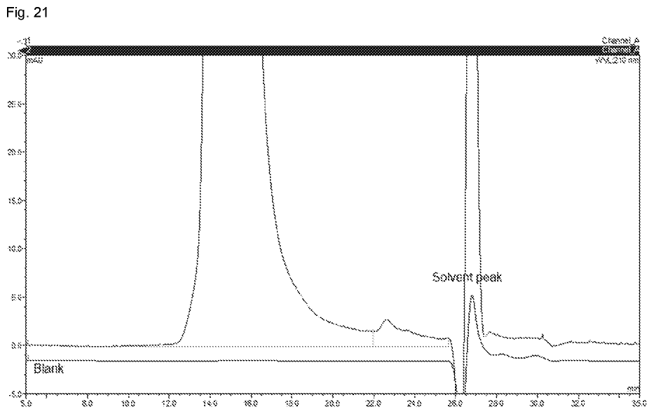

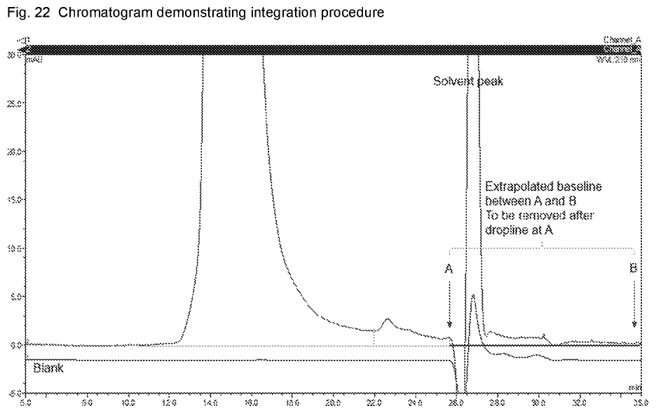

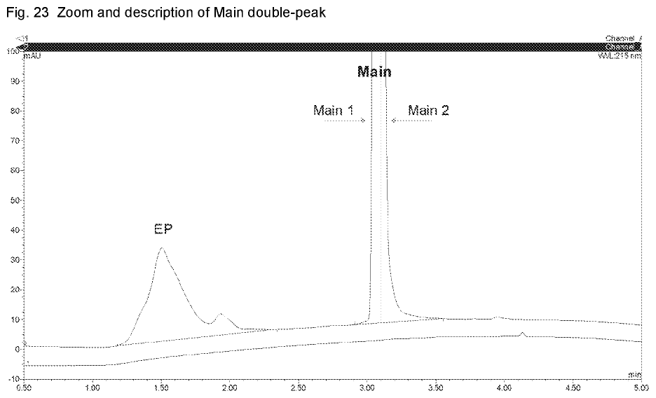

### System Suitability Test (SST) Requirements

| | |
|---|---|
| Visual | The peak pattern of the marker proteins corresponds |
| assessment | to the comparison chromatograms in FIG. 21 or |
| of MWM | FIG. 22. (Aprotinin and Histidine may co-elute after |
| solution | some column use). |
| | The quality of the individual molecular weight |
| | marker proteins may vary and additional small peaks |
| | may be observed. |
| Resolution | The peak resolution R is calculated for the first and |
| | last MWM solution injections to assess the column |
| | performance (see FIG. 23). |
| | Requirement: R must be ≥ 2.0 |
| | $R = \frac{{H}_{AV}}{{h}_{V}}$ |
| | H_{AV}: Peak height of Carbonic anhydrase |
| | h_{V}: Height of valley between Ovalbumin and |
| | Carbonic anhydrase |
| Tailing factor | The tailing factor TF at 10% peak height is |
| | calculated for all reference injections to assess the |
| | separation performance (bracketing approach). See |
| | FIG. 14 for illustration. |
| | Requirement: TF must be ≤ 1.55. |
| | $T F = \frac{A + B}{2 \times A}$ |
| | A: Distance from the center line of the peak max to |
| | the front slope (min) |
| | B: Distance from the center line of the peak max to |
| | the back slope (min) |
| Interference | No interfering peak detected in the blank (diluent) |
| | with a signal height ≥ LOQ signal height, in the |
| | integrated range of the chromatogram of the sample |
| | (approx. 12-26 min). |
| | The blank labeled ²⁾ above should be used. |
| LOQ (1.0%) | Signal-to-noise ratio (S/N) for the lubricin peak in |
| | the LOQ solution must be ≥ 10. Calculation: |
| | $\frac{S}{N} = \frac{2 H}{h}$ |
| | H: Height of the lubricin peak |
| | h: Height of the background noise in the |
| | chromatogram observed over a distance equal to five |
| | times the width at half-height of the peak in front |
| | and after the peak in the chromatogram or in the |
| | blank run. |
| | Example for Chromeleon™ software settings |
| | For Chromeleon™ chromatography data system |
| | version 6.8, the blank injection before LOQ solution |
| | can be used for the calculation of the noise over a |
| | range of 5 times the peak width at half peak height, |
| | within the time window of the lubricin peak in the |
| | LOQ solution injection. |
| | In the SST-Properties window set “Parameter Input |
| | for ‘Signal to Noise Ratio’ ” as 5 times of the “Peak |
| | Width at 50% Height”. |
| Visual | Peak profiles of all reference solution injections must |
| assessment | be visually comparable within the sequence and |
| of reference | comparable to the example chromatograms of |
| | reference in FIG. 15 and FIG. 16. The requirement is |
| | linked to the reference in use. |
| Assay precision | For assay only: the relative standard deviation (Srel) |
| | of all reference injections is ≤ 3.0% (total peak area) |
| | for n = 6 (bracketing approach). |
| Consistency of | The total peak area (aggregate, main variant and |
| dilution | fragments) of the sample injections does not differ |
| (concentration | more than 20% from the first reference injection. |
| range) | |
| | $0.8 \leq \frac{{a}_{sample}}{{a}_{ref}} \leq 1.2$ |
| | aₛₐₘₚₗₑ: total peak area for each individual sample |
| | injection (mAU * min) |
| | a_{ref}: total peak area for the first reference injection |
| | (mAU * min) |

### Evaluation

The baseline setting as described below is established because putative fragments of lower-molecular weight (especially for samples at stressed conditions) were eluting close to the solvent peak but could not be fully resolved. By the below procedure such fragments are more correctly accounted and calculated for. The peak “asymmetry” of main peak on its trailing side is foremost due to actual sample content and less due to peak tailing related e.g. to sample adsorption.

I: Draw one baseline from the onset of main peak (including tentative aggregate peaks) and across and beyond the solvent peak as to project an overall straight baseline. The end-point of the baseline is set on a par/level as the starting-point, or an approximate similar level. The baseline across and beyond the solvent peak is named “extrapolated”. See FIG. 22.

II: After the above baseline projection a drop-line is set at the solvent peak as indicated with an “A” in FIG. 22. After drop-line setting the extrapolated baseline and solvent peak and beyond is removed as to not be included in the area calculation (i.e. excluded from). For final result after the removal of the extrapolated baseline see FIG. 21.

### Peak Integration and Numbering

If more than one fragment peak exists, separate the peaks from each other and from main peak (monomer), by an orthogonal split at the minimum of the valley separating them. If a fragment peak is closely associated to the main peak and resolved by a shoulder only a split may be set at the inflection point of the shoulder. One split only should be set between main peak and aggregate peaks. Aggregates are not reported but integrated for the calculation of total area and separated from main peak for purity determination. Alternatively, no split is set between the monomer peak and aggregate peaks. Aggregates and monomer peak are instead considered as one single peak, called the “Main peak”.

Do not integrate peaks from the blank, if existent, for integration of reference, LOQ and sample solutions.

Fragment peaks are identified according to their elution time relative main peak. Peaks eluting after the main peak/monomer are assigned to fragments/degradation products (named DPx).

### Purity Calculation

Determine the peak area (mAU*min) of all integrated peaks (main+fragments+aggregates) (aggregates are integrated as one unit, no split between individual aggregate peaks if existent).

Alternatively, determine the peak area (mAU*min) of Main peak (aggregates and monomer peak are integrated as one unit, no split between these peaks). In case purity is evaluated in parallel with assay evaluation (3 injections), the mean of the 3 injections is taken for purity calculation.

Calculate for each sample the area percentage (% P) of each fragment peak and main peak. Calculate the sum (%) of relative peak areas of fragments

Peaks<1.0% (LOQ) are not included in the calculation of the sum fragments

The % P of each peak is calculated according to the following formula:

$$\% P = \frac{{a}_{rs}}{{a}_{t}} \times 100$$

aᵣₛ: Peak area of a specific peak in the sample solution chromatogram (mAU*min)

aₜ: Total peak area of the sample solution chromatogram (mAU*min). All integrated peaks (including aggregate peaks) are included.

### Elution Time Difference

Elution time difference defined as Δ (delta) time difference of main peak of measured sample versus main peak of reference standard (mean elution time of first and last reference in the sequence), i.e. Time main peak reference (average, min)−Time main peak sample (min).

Assay calculation

### Drug Substance

Compare the mean total peak area of all reference solution injections (n=6; Aᵣ) (before and after the samples in the sequence, or in addition in-between if more than 15 samples as exemplified above) with the mean total peak area of each sample injected (Aₛ). Alternatively, for more than 15 sample injections, the bracket approach is applied. For each bracket, a mean of total peak area of the reference (n=6) (Aᵣ₁ for the first bracket and Aᵣ₂ for the second bracket) is used to calculate the assay.

Calculate the sample concentration (mg/mL) according to the following formula:

$$Cs = Cr \frac{As \times Ds}{Ar \times Dr}$$

Aᵣ: mean total peak area of all reference solution injections (n=6)

Aₛ: mean total peak area of each sample injected (n=3)

Cs: concentration of the undiluted sample (mg/mL)

Cr: concentration of the undiluted reference (mg/mL)

Ds: sample dilution factor (if no dilution factor then Ds=1)

Dr: reference dilution factor (if no dilution factor then Dr=1)

### Drug Product

Compare the mean total peak area of all reference solution injections (n=6; Aᵣ) (before and after the samples in the sequence, or in addition in-between if more than 15 samples as exemplified above) with the mean total peak area of each sample injected (As). Alternatively, for more than 15 sample injections, the bracket approach is applied. For each bracket, a mean of total peak area of the reference (n=6) (Aᵣ₁ for the first bracket and Aᵣ₂ for the second bracket) is used to calculate the assay.

Calculate the percentage (%) of declared content according to the following formula:

$$\% of declared content = \frac{Cr \times As \times Ds}{CL \times Ar \times Dr} \times 100$$

Aᵣ: mean total peak area of all reference solution injections (n=6)

Aₛ: mean total peak area of each sample injected (n=3)

CL: theoretical concentration (mg/mL) of the drug product of the active pharmaceutical ingredient (lubricin)

Cr: concentration of the undiluted reference (mg/mL) Ds: sample dilution factor (if no dilution factor then Ds=1) Dr: reference dilution factor (if no dilution factor then Dr=1)

### Reporting

Purity: report the peak area percentage (% P) of the monomer (main peak) and the sum (%) of fragments/degradation products (DPs).

Alternatively, purity is reported as the peak area percentage (% P) of the main peak (aggregates and monomer peak as a single unit) and the sum (%) of fragments/degradation products (DPs).

Report for information the % P of each fragment/DP peak≥1.0% (LOQ) together with its corresponding relative elution time (relative to the monomer).

Report for information the A (delta) time difference for each sample main peak versus reference main peak (mean value) rounded to two decimals, X.XX min.

Assay:

Drug substance: report the result per sample in mg/mL (e.g., with two decimals).

Drug product: report the result as percentage (%) of the declared content (e.g., with one decimal).

### Example 5

The following assays were used and can be used for analyzing solutions purified using the methods provided herein.

### Analytical Assay: Assay and Purity by Reversed Phase Chromatography (RPC)

Lubricin is resolved as two major groups of peaks by RPC, referred to as an early- and main peaks, respectively. The peak area of the two major groups of peaks versus the total peak area defines purity expressed as relative peak area percentage.

The assay is defined by a relative comparison of the average total peak area of a sample to the average total peak area of the bracketing injections of reference solutions.

The method is applicable for lubricin drug substance (DS) and drug product (DP) generally referred to as “sample”.

The following solutions are used:

| | |
|---|---|
| Mobile phase A | 90% water/10% ACN/0.1% TFA/0.3% PEG, all (v/v) |
| | e.g. mix 900 mL water, 100 mL ACN, 1 mL TFA and |
| | 3 mL PEG300. |
| Mobile phase B | 10% water/90% ACN/0.1% TFA/0.3% PEG, all (v/v) |
| | e.g. mix 100 mL of water, 900 mL of ACN, 1 mL |
| | TFA and 3 ml PEG300. |
| Sample diluent | 0.2% (v/v) TFA in water |
| | e.g. mix 0.02 mL TFA with 10 mL water |
| | or |
| | 10 mM sodium phosphate/140 mM sodium chloride/ |
| | 0.02% (w/v) polysorbate 20 (PS20), pH 7.0. |

TFA = trifluoroacetic acid
IPA = isopropanol
ACN = acetonitrile
PEG300 = poly(ethylene glycol) 300

The following chromatographic column, conditions, and gradient can be used.

| | |
|---|---|
| Column | Poroshell 300 SB-C8 5 μm; 2.1 mm × 75 mm |
| | (Agilent #660750-906) or equivalent |

Conditions

| | |
|---|---|
| Flow rate | 2.0 mL/min |
| Maximum pressure | 400 bar (approx 6000 psi) |
| Detection | 215 nm |
| | Chromeleon ™ settings: Step: Auto (or 0.05 s)/ |
| | Average: “on” |
| Column temperature | 70° C. |
| | Chromeleon ™ settings: Temperature delta: 2° C. |
| Injection volume | 40 μL |
| Autosampler | 5° C. |
| temperature | Chromeleon ™ settings: Lower limit: 4° C./Upper |
| | limit: 8° C. |
| Run time | 10.0 minutes |

Solvent gradient

| Time [min] | Mobile phase A, % | Mobile phase B, % |
|---|---|---|
| 0.0 | 100 | 0 |
| 0.5 | 100 | 0 |
| 6.9 | 10 | 90 |
| 7.0 | 0 | 100 |
| 8.5 | 0 | 100 |
| 8.6 | 100 | 0 |
| 10 | 100 | 0 |

Test procedure
Test solutions

| | |
|---|---|
| Reference | Dilute lubricin reference with sample diluent to a |
| solution | concentration of 0.15 mg/mL. Pipette the sample diluent |
| (0.15 mg/mL) | first, then the aliquot of reference and mix. This is named |
| | reference solution |
| Sample | If sample concentration is >0.15 mg/mL dilute sample to |
| solution | 0.15 mg/mL with sample diluent. Pipette the sample diluent |
| (0.15 mg/mL) | first, then the aliquot of sample and mix. This is named |
| | sample solution. |
| | For concentrations <0.15 mg/mL samples are injected tel |
| | quel (no dilution). |
| LOQ solution | Dilute in two steps the lubricin reference solution to obtain |
| (1.0%, | a final lubricin concentration (LOQ solution) of 1.5 μg/mL. |
| 1.5 μg/mL | e.g. first, dilute 25 μL of the reference solution at 0.15 |
| lubricin) | mg/mL with 225 μL sample diluent. This is named solution |
| | A (15 μg/mL lubricin). |
| | e.g. second, dilute 25 μL of solution A with 225 μL sample |
| | diluent. This is named LOQ solution (1.5 μg/mL). |
| Blank | 50% (v/v) sample diluent/50% (v/v) isopropanol (IPA). |
| | This is named blank solution. Or use Sample diluent. |

Sequence of injections
Execute the sequence as follows. The reference is injected in the beginning and end of the sequence. The example given is for up to ten (10) samples. If more than ten samples are injected a blank should be run after each 10^{th} sample injection.

| Sample name | No. of injections |
|---|---|
| Blank solution | 3 |
| Reference solution | 1 |
| Blank solution | 1 |
| LOQ solution | 1 |
| Sample 1 solution | 1 |
| . . . | 1 per sample |
| Sample 10 solution | 1 |
| Blank solution | 1 |
| Reference solution | 1 |
| * | * |

* If more than ten (10) samples are to be injected a blank solution should be injected after each 10th sample injection (bracketing approach). The reference should be injected first and last in the sequence

### System Suitability Test (SST)

SST is carried out before each test series. Proceed to sample evaluation only if all SST requirements are accepted.

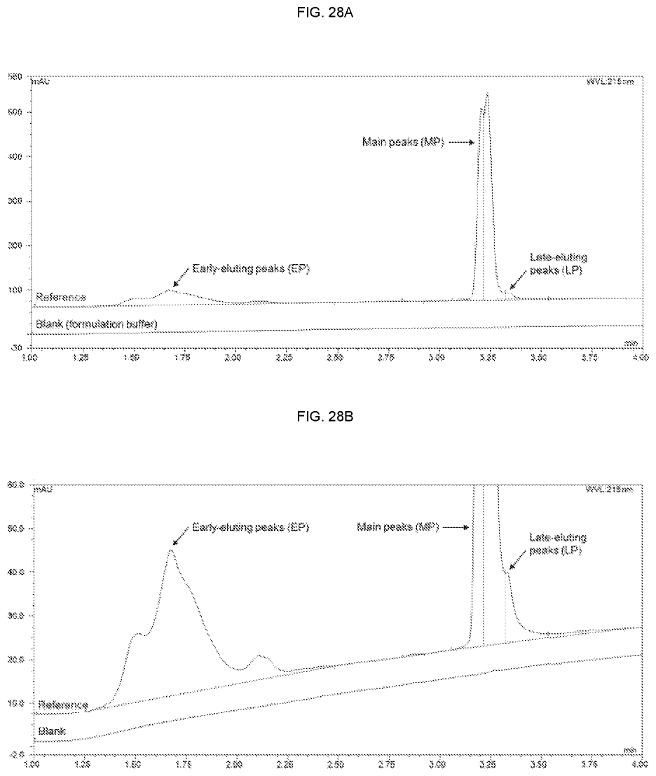

### SST Requirements

| | |
|---|---|
| Reference | Peak profiles of all reference solution injections |
| appearance | must be visually comparable within the sequence |
| (visual assessment | and comparable to the chromatographic profile in |
| of reference) | the example chromatogram in FIG. 28A and FIG. |
| | 28B. The appearance requirement applies only if |
| | the same reference batch is used. |
| Sample | When Assay and Purity are done for the same |
| appearance | sample, the three individual sample preparations |
| (visual assessment | (n = 3) must be visually comparable to each other. |
| of sample) | |
| Specificity | No interfering peak detected in the blank solution |
| | injected just before the LOQ solution, with a |
| | signal height ≥ LOQ signal height in the |
| | integrated range (approx. 1-4 minutes) of the |
| | chromatogram of the sample. |
| LOQ (1.0%) | Signal-to-noise ratio (S/N) for the lubricin peak |
| | in the LOQ solution must be ≥ 10. Calculation: |
| | $\frac{S}{N} = \frac{2 H}{h}$ |
| | H: height of the lubricin peak |
| | h: height of the background noise in the |
| | chromatogram observed over a distance equal to |
| | at least five times the width at half-height of the |
| | peak in front (optionally, and after) the peak in |
| | the chromatogram or in the blank run. |
| | Note: |
| | on the Chromeleon™ chromatography system, |
| | use the following formula for the calculation of |
| | the S/N ratio: (peak.height * 2)/ |
| | (chm.noise(peak.start_time − 0.5 − |
| | (peak.width(50) * 2), peak start_time − 0.5)). |
| | Alternatively, for Chromeleon™ chromatography |
| | data system version 6.8, the third blank injection |
| | of the sequence can be used for the calculation of |
| | the noise over a range of at least 5 times the peak |
| | width at half peak height, within the defined time |
| | window of 2.0 min to 2.5 min. |
| Consistency of | Limit for total peak area (mAU * min) |
| dilution | reproducibility: |
| | $0.8 \leq \frac{{a}_{sample}}{{a}_{ref}} \leq 1.2$ |
| | a_{ref}: total peak area of the first reference injection |
| | aₛₐₘₚₗₑ: total peak area for each individual sample |
| | injection |
| Assay precision | The relative standard deviation (Srel) of all |
| (for Assay only) | reference injections is ≤ 3.0% (total peak area) |
| | for n = 6 (bracketing approach). |
| | The relative standard deviation (Srel) of all |
| | sample injections is ≤ 2.0% (total peak area) for |
| | n = 3. |

### Evaluation

Draw a baseline from the first eluting peak to the last eluting peak in overlay to the blank solution in the approximate range of 1 to 4 minutes encompassing early-eluting peaks (EPs), main peak and tentative late-eluting peaks (LPs).

Alternatively, draw baselines similar to FIG. 28A and FIG. 28B. The first eluting peak (EP), or group of peaks, and the main peak contain separate baselines. The baselines are drawn in relation to the blank solution in the approximate range of 1 to 4 minutes encompassing early-eluting peaks (EP=, main peaks and tentative late-eluting peaks (LP).

### Peak Integration and Numbering

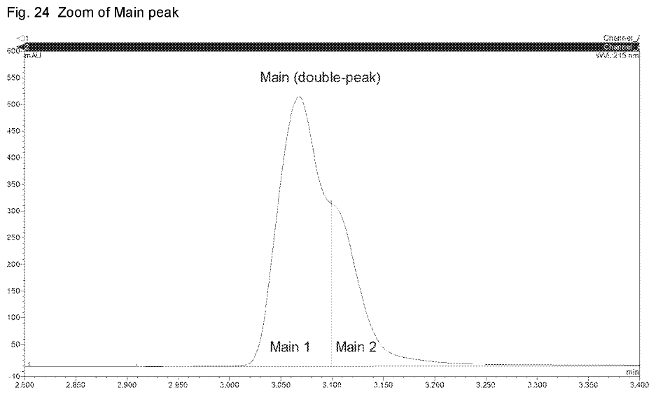

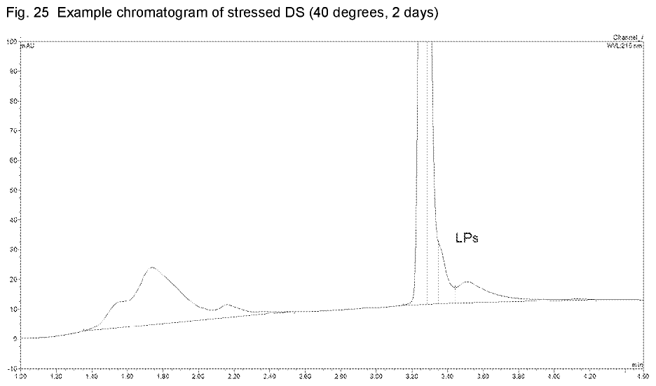

Major peaks can be separated by an orthogonal split at the minimum of the valley separating them or at inflection points (see e.g. FIG. 24 and FIG. 25). Do not integrate solvent/injection peaks or peaks originating from the blank solution, if any.

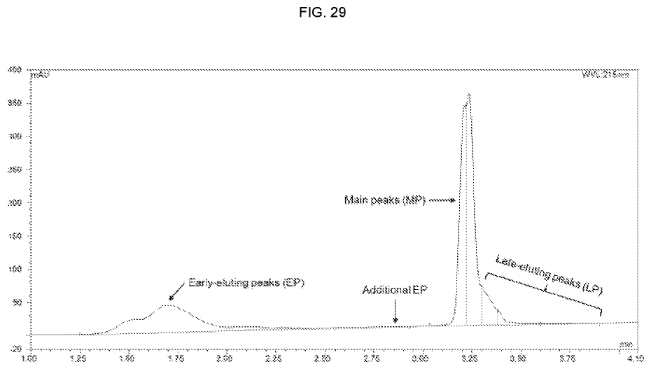

Peaks are identified according to their retention time relative to the main peak. Peaks eluting before the main peak are assigned to early-eluting peaks (named EP) and peaks eluting after the main peak to late-eluting peaks (named LP). The EP in the current reference standard constitute one major but heterogeneous peak. The EP peak is integrated as one unit, see FIG. 24 and FIG. 23. If additional EPs are detected eluting ahead of the major EP, or in-between the major EP and main peak, those peaks should be integrated separately from the major EP by an orthogonal split at the minimum of the valley separating them, or with a separated and independent baseline (see example of a stressed sample in FIG. 29). A similar integration principle applies to LP which should be integrated as a peak or group of peaks if resolved from the main peak either by a valley or inflection point (see FIG. 29 where several groups of LP are integrated).

The main peak of the reference standard is frequently resolved as a double-peak, two incompletely resolved major peaks. The main double-peak should be split and integrated (by a dropline, resulting in Main 1 and Main 2) also if not resolved by a valley but at least an inflection point, see FIG. 24. Alternatively, the main double-peak can be integrated as two single peaks.

Purity calculation

When Assay and Purity are performed on the same sample, use the first sample replicate only to assess the Purity.

Determine the peak area (mAU*min) of all integrated peaks.

The percentage, % P, of EP and Main peak (sum of double-peak, if existent) are calculated according to the following formula:

$$\% P = \frac{{a}_{rs}}{{a}_{t}} \times 100$$

aᵣₛ: Peak area of EP and Main, respectively, in the chromatogram (mAU*min)

aₜ: Total peak area of the sample solution chromatogram (mAU*min) i.e. the sum area of all integrated peaks (EPs, Main and LPs).

Alternatively, the percentage, % P of EP, sum of main peaks and LP are calculated according to the following formula:

$$\% P = \frac{{a}_{rs}}{{a}_{t}} \times 100$$

aᵣₛ: Peak area of EP, sum of main, and LP, respectively, in the chromatogram (mAU*min)

aₜ: Total peak area of the sample solution chromatogram (mAU*min) i.e. the sum area of all integrated peaks (EPs, Main and LPs).

Assay calculation

### Drug Substance

Compare the mean total peak area of all reference solution injections n=6 (Aᵣ) (before and after the samples in the sequence with the mean total peak area of each sample injected

(Aₛ)). For more than 12 sample injections (corresponding to 4 samples in triplicates) the bracket approach is applied. For each bracket a mean of total peak area of the reference (n=6) (Aᵣ₁ for the first bracket and Aᵣ₂ for the second bracket) is used to calculate the assay.

Calculate the sample concentration (mg/mL) according to the following formula:

$$Cs = Cr \frac{As \times Ds}{Ar \times Dr}$$

Aᵣ: mean total peak area of all reference solution injections (n=6)
Aₛ: mean total peak area of each sample injected (n=3)
Cs: concentration of the undiluted sample (mg/mL)
Cr: concentration of the undiluted reference (mg/mL)
Ds: sample dilution factor (if no dilution factor then Ds=1)
Dr: reference dilution factor (if no dilution factor then Dr=1)

### Drug Product

Compare the mean total peak area of all reference solution injections n=6 (Aᵣ) (before and after the samples in the sequence with the mean total peak area of each sample injected (Aₛ)). For more than 12 sample injections (corresponding to 4 samples in triplicates) the bracket approach is applied. For each bracket a mean of total peak area of the reference (n=6) (Aᵣ₁ for the first bracket and Aᵣ₂ for the second bracket) is used to calculate the assay.

Calculate the percentage (%) of declared content according to the following formula:

$$\% of declared content = \frac{Cr \times As \times Ds}{CL \times Ar \times Dr} \times 100$$

Aᵣ: mean total peak area of all reference solution injections (n=6)
Aₛ: mean total peak area of each sample injected (n=3)
CL: theoretical concentration (mg/mL) of the drug product of the active pharmaceutical ingredient (ECF843)
Cr: concentration of the undiluted reference (mg/mL)
Ds: sample dilution factor (if no dilution factor then Ds=1)
Dr: reference dilution factor (if no dilution factor then Dr=1)

### Retention Time Difference

Retention time difference defined as A (delta) time difference of main peak of measured sample versus main peak of reference standard (mean retention time of first and last reference in the sequence), i.e. Time main peak sample (min)−Time main peak reference (average, min).

### Reporting

Purity: report the sum percentage (% P) of major EP peak and main peak (sum of main double-peak 1 and 2, if existent)

Report for information the % P of each peak≥1.0% (LOQ), including the major EP and Main (sum of main peaks 1 and 2).

Report for information the percentage (%) of the two peaks constituting the main peak 1 and 2 (double-peak, if existent).

Report for information the A (delta) time difference for each sample main peak versus reference main peak (mean value) rounded to two decimals, X.XX min.

Alternatively, the following reporting criteria are used.

Purity: The Purity (%) is defined as the sum of: the EPs (%) and the sum of main peaks (%). Note that all early-eluting peaks (major heterogeneous peak integrated as one unit (EP), and additional early-eluting peaks (EP1, EP2, etc.)) are considered for the Purity (%).

Report the Purity (%), the EP (%), the individual main peaks (%) and the sum of main peaks (%).

Report for information the % P of each peak≥1.0% (LOQ).

Assay:

For drug substance: report the result per sample in mg/mL.

For drug product: report the result as percentage (%) of the declared content.

### Example 6

### Glycosylation Assays—Determining Amounts of O-glycans and N-glycans

O-glycans of drug substance (DS) batches, including the clinical DS batch purified using the method described in Example 2 above, were chemically released (reductive beta-elimination) and derivatized (permethylation) after drug substance batches were first subjected to disulfide reduction/alkylation and trypsin digestion. The O-glycans were profiled by reversed-phase chromatography coupled to electrospray ionization mass spectrometry (ESI-MS) and detection/identification was performed via MS or tandem mass spectrometry (MS/MS).

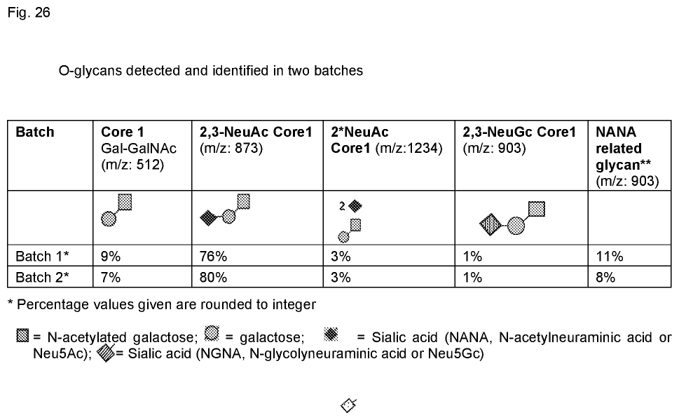

The major O-glycan species detected in both DS batches included: monosialylated (NANA) Gal-GalNAc (core 1 structure; initial DS batch, 76%; clinical DS batch, 80%), Gal-GalNAc (initial DS batch, 9%; clinical DS batch, 7%), NANA** related glycan (initial DS batch, 11%; clinical DS batch, 8%), disialylated (2*NANA) Gal-GalNAc (initial DS batch, 3%; clinical DS batch, 3%) and NGNA (Nglycolyneuraminic) Gal-GalNAc (initial DS batch, 1%; clinical DS batch, 1%). The composition and relative abundance of O-glycans was comparable between the initial and clinical DS batches (See FIG. 26: initial DS batch=Batch 1; clinical DS batch=Batch 2). A small amount of N-glycolyneuraminic (NGNA; around 1% or less) was detected in both batches. A NANA related glycan was also detected, and may be an oxidized form of monosialylated (NANA) Gal-GalNAc. The NANA related glycan may be an artifact of sample preparation.

N-glycans of drug substance (DS) batches were enzymatically released (PNGaseF), reduced (sodium borohydride) and derivatized (permethylation) after first being subjected to disulfide-bond reduction/alkylation and trypsin digestion. The N-glycans were measured and identified by MALDI-TOF mass spectrometry. The major N-glycans detected and identified corresponded to the high-mannoses —Man-5, Man-6 and Man-7—which were present at similar levels in both batches. Man-6 was the most prevalent N-glycan, followed by Man-5, and Man-7.

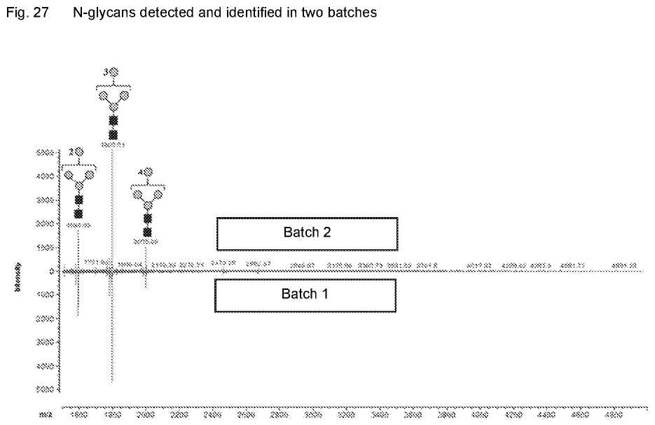

The type/isoform and relative abundance of O-glycans and the N-glycans of the two batches, including the clinical DS batch made using methods described herein, were comparable (See FIG. 26 and FIG. 27).

### INCORPORATION BY REFERENCE

The entire disclosure of each of the patent documents and scientific articles cited herein is incorporated by reference for all purposes.

### EQUIVALENTS

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A method of purifying a recombinant lubricin glycoprotein, the method comprising the steps of subjecting a cell culture harvest containing said lubricin glycoprotein to: a multimodal cation exchange chromatography (MCC), a multimodal anion exchange chromatography (MAC), and a hydrophobic interaction chromatography (HIC), which are performed in any order.

2. The method of claim 1, wherein the steps are performed in the following order: a) MCC, b) MAC, and c) HIC.

3. The method of claim 2, wherein the method further comprises prior to step a), contacting cells in culture with MgCl₂ and an endonuclease, and harvesting the cells to obtain said cell culture harvest.

4. (canceled)

5. The method of claim 2, wherein prior to step a), the cell culture harvest is contacted with MgCl₂ and an endonuclease.

6-8. (canceled)

9. The method of claim 1, further comprising a step of virus inactivation after the multimodal anion exchange chromatography (MAC) step and before the hydrophobic interaction chromatography (HIC) step.

10-11. (canceled)

12. The method of claim 9, the method further comprising a depth filtration step prior to the hydrophobic interaction chromatography (HIC) step.

13. (canceled)

14. The method of claim 1, the method further comprising a virus removal step after the hydrophobic interaction chromatography (HIC) step.

15. (canceled)

16. The method of claim 14, the method further comprising an ultrafiltration step after the virus removal step.

17. The method of claim 14, the method further comprising a virus inactivation step after the virus removal step.

18. (canceled)

19. The method of claim 17, the method further comprising an ultrafiltration step after the virus inactivation step.

20-22. (canceled)

23. The method of claim 1, wherein the recombinant lubricin glycoprotein comprises the amino acid sequence of amino acid residues 25-1404 of SEQ ID NO:1 or 2.

24. The method of claim 1, wherein at least 30% of the molecular weight of the recombinant lubricin glycoprotein is from glycosidic residues.

25. The method of claim 1, wherein at least 90% of O-glycosylation of the lubricin glycoprotein is core 1 glycosylation.

26. The method of claim 1, wherein the lubricin glycoprotein comprises O-glycan species, wherein the O-glycan species comprise about 7% or more Gal-GalNAc, about 80% or more 2,3-NeuAc Core 1, about 3% or more 2*NeuAc Core 1, and about 1% or more 2,3-NeuGc Core 1.

27. The method of claim 1, wherein the lubricin glycoprotein comprises about 50 μg or more NANA per mg of the lubricin glycoprotein.

28. The method of claim 1, wherein the lubricin glycoprotein comprises about 10 μg or less NGNA per mg of the lubricin glycoprotein.

29. The method of claim 1, wherein the lubricin glycoprotein comprises about 100 μg or more Gal per mg of the lubricin glycoprotein.

30. The method of claim 1, wherein the lubricin glycoprotein comprises about 100 μg or more GalNAc per mg of the lubricin glycoprotein.

31. A recombinant lubricin glycoprotein obtained by the method according to claim 1.

32. A pharmaceutical composition comprising the recombinant lubricin glycoprotein according to claim 31 and a pharmaceutically acceptable excipient.

33-43. (canceled)

44. A method for treating an ocular surface disorder, the method comprising a step of administering the pharmaceutical composition of claim 32 to a patient.

45. (canceled)


SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 3 - protein, 7 aa, Homo sapiens
KEPAPTT